(19) United States
(12) Patent Application Publication
(10) Pub. No.: US 2013/0210724 A1
(43) Pub. Date: Aug. 15, 2013

(54) Cyclodextrin-Based Polymers for Therapeutic Delivery

(71) Applicant: Cerulean Pharma Inc., (US)

(72) Inventors: Oliver S. Fetzer, Needham, MA (US); Jungyeon Hwang, Lexington, MA (US); Patrick Lim Soo, Somerville, MA (US); Pei-Sze Ng, Cambridge, MA (US); Sonke Svenson, Arlington, MA (US); Cissy Young, Waban, MA (US)

(73) Assignee: Cerulean Pharma Inc., Cambridge, MA (US)

(21) Appl. No.: 13/861,627

(22) Filed: Apr. 12, 2013

Related U.S. Application Data
(63) Continuation of application No. 13/452,132, filed on Apr. 20, 2012, now abandoned.
(60) Provisional application No. 61/522,901, filed on Aug. 12, 2011.
(60) Provisional application No. 61/477,905, filed on Apr. 21, 2011.

(51) Int. Cl. A61K 47/48

(52) U.S. Cl. CPC A61K 47/4823
USPC 514/7.8; 514/10.3; 514/12.4; 514/9.7; 514/11.8; 514/20.9; 530/322; 530/345; 530/301; 530/317

(57) ABSTRACT

Described herein are CDP-therapeutic peptide conjugates, therapeutic delivery systems comprising CDP-therapeutic peptide conjugates, compositions comprising CDP-therapeutic peptide conjugates, dosage forms comprising CDP-therapeutic peptide conjugates, and kits comprising CDP-therapeutic peptide conjugates. Also disclosed are methods of using (e.g., to treat a disorder) the CDP-therapeutic peptide conjugates, therapeutic delivery systems comprising CDP-therapeutic peptide conjugates, compositions comprising CDP-therapeutic peptide conjugates, dosage forms comprising CDP-therapeutic peptide conjugates, and kits comprising CDP-therapeutic peptide conjugates.

This application claims priority to U.S. Provisional Application No. 61/477,905, filed Apr. 21, 2011 and U.S. Provisional Application No. 61/522,901, filed Aug. 12, 2011, the disclosures of each of which are hereby incorporated by reference in their entireties.

### BACKGROUND OF INVENTION

The delivery of a therapeutic peptide (TP) with controlled release of the therapeutic peptide is desirable to provide optimal use and effectiveness. Controlled release cyclodextrin-based polymer (CDP) systems may increase the efficacy of the therapeutic peptide and minimize problems with patient compliance.

### SUMMARY OF INVENTION

Described herein are CDP-therapeutic peptide conjugates, therapeutic delivery systems comprising CDP-therapeutic peptide conjugates, compositions comprising CDP-therapeutic peptide conjugates, dosage foams comprising CDP-therapeutic peptide conjugates, and kits comprising CDP-therapeutic peptide conjugates. Also disclosed are methods of using (e.g., to treat a disorder) the CDP-therapeutic peptide conjugates, therapeutic delivery systems comprising CDP-therapeutic peptide conjugates, compositions comprising CDP-therapeutic peptide conjugates, dosage forms comprising CDP-therapeutic peptide conjugates, and kits comprising CDP-therapeutic peptide conjugates. For example, the CDP-therapeutic peptide conjugates can be used in the treatment of cancer, inflammatory disorders (e.g., an inflammatory disorder that includes an inflammatory disorder caused by, e.g., an infectious disease), autoimmune disorders, cardiovascular diseases, kidney disease, metabolic disorders, and infectious disease. Also disclosed are methods of making the CDP-therapeutic peptide conjugates.

In one aspect, the disclosure features a CDP-therapeutic peptide conjugate. In an embodiment, the CDP-therapeutic peptide conjugate comprises therapeutic peptide molecules coupled, e.g., via a linker such as a linker described herein, to a CDP moiety, e.g., a CDP described herein. In an embodiment, the CDP-therapeutic peptide conjugate comprises a therapeutic peptide coupled via a linker shown herein. In one embodiment, the therapeutic peptide is a peptide described herein. In one embodiment, the CDP is not biodegradable. In one embodiment, the CDP is biodegradable. In one embodiment, the CDP is biocompatible.

In one aspect, the disclosure features a method of treating a disorder in a subject in need thereof, comprising administering to the subject a CDP-therapeutic peptide conjugate in an amount effective to treat the disorder. In an embodiment, the CDP-therapeutic peptide conjugate comprises therapeutic peptide molecules coupled, e.g., via a linker such as a linker described herein, to a CDP moiety, e.g., a CDP described herein. In an embodiment, the CDP-therapeutic peptide conjugate comprises a therapeutic peptide coupled via a linker shown herein. In one embodiment, the therapeutic peptide is a peptide described herein. In one embodiment, the CDP is not biodegradable. In one embodiment, the CDP is biodegradable. In one embodiment, the CDP is biocompatible.

In one aspect, the disclosure features a method of treating a disorder in a subject in need thereof, comprising administering to the subject a CDP-therapeutic peptide conjugate in an amount effective to treat the disorder, wherein the CDP-therapeutic peptide is of the formula:

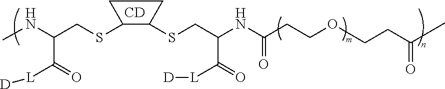

wherein each L is independently a linker or absent and each D is independently a therapeutic peptide, a prodrug thereof, or absent, and wherein the group

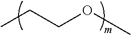

has a Mw of 5 kDa or less (e.g., 3.4 kDa) and n is at least 4, 5, 6, 7, 8, 9, 10, 11, 12, 13, 14, 15, 16, 17, 18, 19 or 20, provided that the polymer comprises at least one therapeutic peptide, thereby treating the subject. In one embodiment, the therapeutic peptide is a peptide described herein. In one embodiment. L is independently an amino acid derivative. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the disorder is cancer, allergies, an inflammatory disease, an auto-immune disease, a cardiovascular disease, a renal disease, or a metabolic disorder. In one embodiment, the subject is a human. In one embodiment, the CDP-therapeutic peptide conjugate is administered by intravenous administration. In one embodiment, the CDP-therapeutic peptide conjugate is administered orally.

In one aspect, the disclosure features a method of treating a disorder in a subject in need thereof, comprising administering to the subject a CDP-therapeutic peptide conjugate, wherein the CDP-therapeutic peptide conjugate comprises a subunit of the following formula:

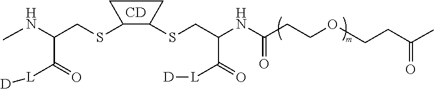

wherein each L is independently a linker and each D is independently a therapeutic peptide, a prodrug thereof and wherein the group

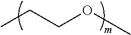

has a Mw of 5 kDa or less (e.g., 3.4 kDa) and n is at least 4, 5, 6, 7, 8, 9, 10, 11, 12, 13, 14, 15, 16, 17, 18, 19 or 20, thereby treating the subject. In one embodiment, the therapeutic peptide is a peptide described herein. In one embodiment, L is independently an amino acid derivative. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the disorder is cancer, allergies, an inflammatory disease, an auto-immune disease, a cardiovascular disease, a renal disease, or a metabolic disorder. In one embodiment, the subject is a human. In one embodiment, the CDP-therapeutic peptide conjugate is administered by intravenous administration. In one embodiment, the CDP-therapeutic peptide conjugate is administered orally.

In one aspect, the disclosure features a CDP-therapeutic peptide conjugate, wherein the CDP-therapeutic peptide conjugate has the following formula:

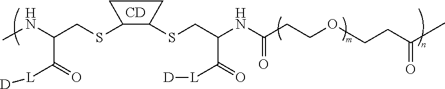

wherein each L is independently a linker or absent and each D is independently a therapeutic peptide, a prodrug thereof, or absent, and wherein the group

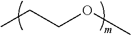

has a Mw of 5 kDa or less (e.g., 3.4 kDa) and n is at least 4, 5, 6, 7, 8, 9, 10, 11, 12, 13, 14, 15, 16, 17, 18, 19 or 20, provided that the polymer comprises at least one therapeutic peptide. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one aspect, a CDP-therapeutic peptide conjugate, wherein the CDP-therapeutic peptide conjugate comprises a subunit of the following formula:

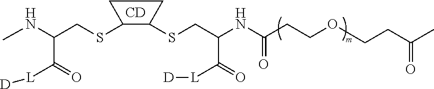

wherein each L is independently a linker and each D is independently a therapeutic peptide, a prodrug thereof and wherein the group

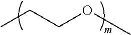

has a Mw of 5 kDa or less (e.g., 3.4 kDa) and n is at least 4, 5, 6, 7, 8, 9, 10, 11, 12, 13, 14, 15, 16, 17, 18, 19 or 20. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one aspect, the disclosure features a therapeutic delivery system comprising a CDP-therapeutic peptide conjugate, wherein the CDP-therapeutic peptide conjugate has the following formula:

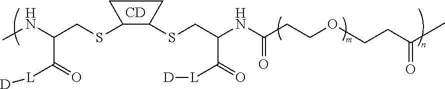

wherein each L is independently a linker or absent and each D is independently a therapeutic peptide, a prodrug thereof, or absent, and wherein the group

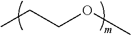

has a Mw of 5 kDa or less (e.g., 3.4 kDa) and n is at least 4, 5, 6, 7, 8, 9, 10, 11, 12, 13, 14, 15, 16, 17, 18, 19 or 20, provided that the polymer comprises at least one therapeutic peptide. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect, the disclosure features a therapeutic delivery system comprising a CDP-therapeutic peptide conjugate, wherein the CDP-therapeutic peptide conjugate comprises a subunit of the following formula:

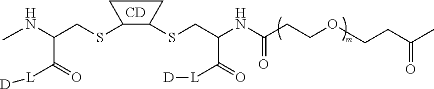

wherein each L is independently a linker and each D is independently a therapeutic peptide, a prodrug thereof and wherein the group

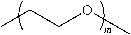

has a Mw of 5 kDa or less (e.g., 3.4 kDa) and n is at least 4, 5, 6, 7, 8, 9, 10, 11, 12, 13, 14, 15, 16, 17, 18, 19 or 20. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect, the disclosure features an inclusion complex comprising a therapeutic delivery system comprising a CDP-therapeutic peptide conjugate, wherein the CDP-therapeutic peptide conjugate has the following formula:

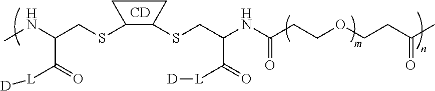

wherein each L is independently a linker or absent and each D is independently a therapeutic peptide, a prodrug thereof, or absent, and wherein the group

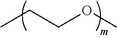

has a Mw of 5 kDa or less (e.g., 3.4 kDa) and n is at least 4, 5, 6, 7, 8, 9, 10, 11, 12, 13, 14, 15, 16, 17, 18, 19 or 20, provided that the polymer comprises at least one therapeutic peptide. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect the disclosure features an inclusion complex comprising a therapeutic delivery system comprising a CDP-therapeutic peptide conjugate, wherein the CDP-therapeutic peptide conjugate comprises a subunit of the following formula:

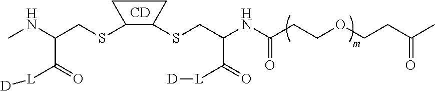

wherein each L is independently a linker and each D is independently a therapeutic peptide, a prodrug thereof and wherein the group

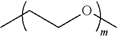

has a Mw of 5 kDa or less (e.g., 3.4 kDa) and n is at least 4, 5, 6, 7, 8, 9, 10, 11, 12, 13, 14, 15, 16, 17, 18, 19 or 20. In one embodiment, the therapeutic peptide is a peptide described herein. In one embodiment, the CDP is not biodegradable. In one embodiment, the CDP is biodegradable. In one embodiment, the CDP is biocompatible.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect, the disclosure features a composition comprising a therapeutic delivery system comprising a CDP-therapeutic peptide conjugate, wherein the CDP-therapeutic peptide conjugate has the following formula:

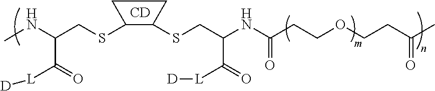

wherein each L is independently a linker or absent and each D is independently a therapeutic peptide, a prodrug thereof, or absent, and wherein the group

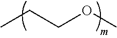

has a Mw of 5 kDa or less (e.g., 3.4 kDa) and n is at least 4, 5, 6, 7, 8, 9, 10, 11, 12, 13, 14, 15, 16, 17, 18, 19 or 20, provided that the polymer comprises at least one therapeutic peptide. In one embodiment, the therapeutic peptide is a peptide described herein. In one embodiment, the CDP is not biodegradable. In one embodiment, the CDP is biodegradable. In one embodiment, the CDP is biocompatible.

In one embodiment, the composition is substantially free of un-conjugated therapeutic peptide. In one embodiment, the composition is a pharmaceutical composition.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect the disclosure features a composition comprising a therapeutic delivery system comprising a CDP-therapeutic peptide conjugate, wherein the CDP-therapeutic peptide conjugate comprises a subunit of the following formula:

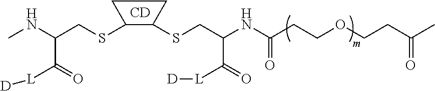

wherein each L is independently a linker and each D is independently a therapeutic peptide, a prodrug thereof and wherein the group

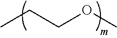

has a Mw of 5 kDa or less (e.g., 3.4 kDa) and n is at least 4, 5, 6, 7, 8, 9, 10, 11, 12, 13, 14, 15, 16, 17, 18, 19 or 20. In one embodiment, the therapeutic peptide is a peptide described herein. In one embodiment, the CDP is not biodegradable. In one embodiment, the CDP is biodegradable. In one embodiment, the CDP is biocompatible.

In one embodiment, the composition is substantially free of un-conjugated therapeutic peptide. In one embodiment, the composition is a pharmaceutical composition.

In one embodiment, each L of the CDP-therapeutic peptide conjugate is independently an amino acid derivative. In one embodiment, at least a portion of the CDP is covalently attached to the therapeutic peptide through a cysteine moiety. In one embodiment, the linker comprises a moiety formed using “click chemistry” (e.g., as described in WO 2006/115547). In one embodiment, the linker comprises an amide bond, an ester bond, a disulfide bond, or a triazole. In one embodiment, the linker comprises a bond that is cleavable under physiological conditions. In one embodiment, the linker is hydrolysable under physiologic conditions or the linker is enzymatically cleavable under physiological conditions (e.g., the linker comprises a disulfide bond which can be reduced under physiological conditions). In one embodiment, the linker is not cleavable under physiological conditions. In one embodiment, at least a portion of the CDP is covalently attached to the therapeutic peptide through the carboxy terminal of the therapeutic peptide. In one embodiment, at least a portion of the CDP is covalently attached to the therapeutic peptide through an amino acid side of the therapeutic peptide.

In one embodiment, the therapeutic peptides are from about 1 to about 100 weight % of the conjugate, e.g., from 1 to about 80 weight % of the conjugate, e.g., from 1 to about 70 weight % of the conjugate, e.g., from 1 to about 60 weight % of the conjugate, e.g., from 1 to about 50 weight % of the conjugate, e.g., from 1 to about 40 weight % of the conjugate, e.g., from 1 to about 30 weight % of the conjugate, e.g., from 1 to about 20 weight % of the conjugate, e.g., from 1 to about 10 weight % of the conjugate.

In one embodiment, the therapeutic delivery system further comprises a counter ion. In one embodiment, the counter ion is a cation. In one embodiment, the counter ion is an anion. In one embodiment the therapeutic delivery system further comprises a surfactant. In one embodiment, the surfactant is a polymer. In one embodiment, the surfactant is PVA. In one embodiment, the surfactant is from about 5 to about 50 weight % of the system, e.g., from about 10 to about 40 weight % of the system, e.g., from about 20 to about 30 weight % of the system.

In one aspect, the disclosure features a method of making a CDP-therapeutic peptide conjugate comprising providing a therapeutic peptide and a CDP and subjecting the therapeutic peptide and CDP to conditions that affect the covalent attachment of the therapeutic peptide to the CDP. In one embodiment, the method is performed in a reaction mixture. In one embodiment, the reaction mixture comprises a single solvent. In one embodiment, the reaction mixture comprises a solvent system comprising a plurality of solvents. In one embodiment, the plurality of solvents are miscible. In one embodiment, at least one of the therapeutic peptide or CDP is attached to an insoluble substrate.

In an embodiment, the CDP-therapeutic peptide conjugate comprises therapeutic peptide molecules coupled. e.g., via a linker such as a linker described herein, to a CDP moiety, e.g., a CDP described herein. In an embodiment, the CDP-therapeutic peptide conjugate comprises a therapeutic peptide coupled via a linker shown herein.

In one embodiment, the CDP-therapeutic peptide conjugate forms a particle (e.g., a nanoparticle). In one embodiment, the inclusion complex comprising a CDP-therapeutic peptide conjugate forms a particle (e.g., a nanoparticle). In some embodiments, the particle has a diameter of less than 500 nm, e.g., less than 300 nm (e.g., the particles in a composition described herein have a Dv90 of less than 500 nm, e.g., less than 300 nm). The nanoparticles generally range in size from 10 to 300 nm in diameter, e.g., 10 to 280, 20 to 280, 30 to 250, 30 to 200, 20 to 150, 30 to 100, 20 to 80, 10 to 80, 10 to 70, 20 to 60 or 20 to 50 nm 10 to 70, 10 to 60 or 10 to 50 nm diameter. In one embodiment, the nanoparticle is 20 to 60 nm in diameter. In one embodiment, the composition comprises a population or a plurality of nanoparticles with an average diameter from 10 to 300 nm, e.g., 20 to 280, 15 to 250, 15 to 200, 20 to 150, 15 to 100, to 80, 15 to 80, 15 to 70, 15 to 60, 15 to 50, or 20 to 50 nm. In one embodiment, the average nanoparticle diameter is from 15 to 60 nm (e.g., 20-60 nm), e.g., the average of the nanoparticles in a composition described herein have a Dv90 of 15 to 60 nm. In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

In one embodiment, the therapeutic peptide conjugated to the CDP is more soluble when conjugated to the CDP, than when not conjugated to the CDP, e.g., when the therapeutic peptide is free from conjugation to a moiety such as a polymer.

In one embodiment, the composition comprises a population, mixture, composition, or plurality of CDP-therapeutic peptide conjugates. In one embodiment, the population, mixture, composition, or plurality of CDP-therapeutic peptide conjugates comprises a plurality of different therapeutic peptide conjugated to a CDP (e.g., two different therapeutic peptides are in the composition such that two different therapeutic peptides are attached to a single CDP; or a first therapeutic peptide is attached to a first CDP and a second therapeutic peptide is attached to a second CDP and both CDP-therapeutic peptide conjugates are present in the composition).

In one aspect, the disclosure features a method of treating a proliferative disorder, e.g., a cancer, in a subject, e.g., a human, the method comprising administering a composition that comprises a CDP-therapeutic peptide conjugate to a subject in an amount effective to treat the disorder, to thereby treat the proliferative disorder. In an embodiment, the CDP-therapeutic peptide conjugate comprises a therapeutic peptide molecule coupled, e.g., via a linker such as a linker described herein, to a CDP described herein.

In one embodiment, the composition is administered in combination with one or more additional anticancer agent, e.g., chemotherapeutic agent, e.g., a chemotherapeutic agent or combination of chemotherapeutic agents described herein, and radiation.

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e advanced or metastatic melanoma), germ cell tumors, ovarian cancer (e.g., advanced ovarian cancer, e.g., advanced fallopian tube or peritoneal cancer), and gastrointestinal cancer.

In one aspect, the disclosure features a method of treating a disease or disorder associated with inflammation, e.g., an allergic reaction or an autoimmune disease, in a subject, e.g., a human, the method comprises: administering a composition that comprises a CDP-therapeutic peptide conjugate to a subject in an amount effective to treat the disorder, to thereby treat the disease or disorder associated with inflammation. In an embodiment, the CDP-therapeutic peptide conjugate comprises a therapeutic peptide molecule coupled, e.g., via a linker such as a linker described herein, to a CDP described herein. In an embodiment, the CDP-therapeutic peptide conjugate comprises a therapeutic peptide molecule, coupled via a linker to a CDP moiety, e.g., a CDP described herein.

In one embodiment, the disease or disorder associated with inflammation is a disease or disorder described herein. For example, the disease or disorder associated with inflammation can be for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatitis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components. In some embodiments, the autoimmune disease is an organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), or Grave's disease.

In another embodiment, a CDP-therapeutic peptide conjugate or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The CDP-therapeutic peptide conjugate, particle or composition may be used to treat chronic hepatitis infection, including hepatitis B and hepatitis C.

In one aspect, the disclosure features a method of treating cardiovascular disease, e.g., heart disease, in a subject, e.g., a human, the method comprising administering a composition that comprises a CDP-therapeutic peptide conjugate to a subject in an amount effective to treat the disorder, to thereby treat the cardiovascular disease. In an embodiment, the CDP-therapeutic peptide conjugate comprises a therapeutic peptide molecule coupled, e.g., via a linker such as a linker described herein, to a CDP described herein. In an embodiment, the CDP-therapeutic peptide conjugate comprises a therapeutic peptide molecule, coupled via a linker to a CDP moiety, e.g., a CDP described herein.

In one embodiment, cardiovascular disease is a disease or disorder described herein. For example, the cardiovascular disease may be cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using CDP-therapeutic peptide conjugates, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. Yet other disorders that may be treated with CDP-therapeutic peptide conjugates, particles, compositions and methods described herein include restenosis, e.g., following coronary intervention, and disorders relating to an abnormal level of high density and low density cholesterol.

In one embodiment, the CDP-therapeutic peptide conjugate, particle or composition can be administered to a subject undergoing or who has undergone angioplasty. In one embodiment, the CDP-therapeutic peptide conjugate, particle or composition is administered to a subject undergoing or who has undergone angioplasty with a stent placement. In some embodiments, the CDP-therapeutic peptide conjugate, particle or composition can be used as a strut of a stent or a coating for a stent.

In one aspect, the disclosure features a method of treating a disease or disorder associated with the kidney, e.g., renal disorders, in a subject, e.g., a human, the method comprises: administering a composition that comprises a CDP-therapeutic peptide conjugate to a subject in an amount effective to treat the disorder, to thereby treat the disease or disorder associated with kidney disease. In an embodiment, the CDP-therapeutic peptide conjugate comprises a therapeutic peptide molecule coupled, e.g., via a linker such as a linker described herein, to a CDP described herein. In an embodiment, the CDP-therapeutic peptide conjugate comprises a therapeutic peptide molecule, coupled via a linker to a CDP moiety, e.g., a CDP described herein.

In one embodiment, the disease or disorder associated with the kidney is a disease or disorder described herein. For example, the disease or disorder associated with the kidney can be for example, acute kidney failure, acute nephritic syndrome, analgesic nephropathy, atheroembolic renal disease, chronic kidney failure, chronic nephritis, congenital nephrotic syndrome, end-stage renal disease, good pasture syndrome, interstitial nephritis, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis.

In some embodiments, the CDP of the conjugate is covalently attached to the therapeutic peptide via a linker. Exemplary linkers include a linker comprising a moiety formed using “click chemistry” (e.g., as described in WO 2006/115547) and a linker that comprises an amide bond, an ester bond, or a triazole. In some embodiments, the linker comprises a bond that is cleavable under physiological conditions. In some embodiments, the linker is hydrolysable under physiologic conditions, the linker is enzymatically cleavable under physiological conditions, or the linker comprises a disulfide bond which can be reduced under physiological conditions.

In some embodiments, the particle further comprises a plurality of additional therapeutic peptides, wherein the additional therapeutic peptides differ from the first therapeutic peptides. In some embodiments, at least a portion of the plurality of the additional therapeutic peptides are attached to at least a portion of the CDP.

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, at least a portion of the therapeutic peptide are chemically modified.

In some embodiments, the composition is chemically stable under ambient conditions for at least 1 day (e.g., at least 7 days, at least 14 days, at least 21 days, at least 30 days). In some embodiments, the composition is chemically stable under conditions comprising a temperature of 23 degrees Celsius and 60, 70, or 80 percent humidity for at least 1 day (e.g., at least 7 days, at least 14 days, at least 21 days, at least 30 days).

In some embodiments, the subject is any of a mouse, rat, dog, or human.

In some embodiments, the composition, when administered to a subject, results in a peak plasma concentration (Cₘₐₓ) that is less than 90, 80, 70, 60, 50, 40, 30, 20, 10, 5, or 1% of that of the Cₘₐₓ of said therapeutic peptide administered free to the subject. In some embodiments, the composition and therapeutic peptide administered free are administered under similar conditions. In some embodiments, the amount of therapeutic peptide in the particle composition administered to the subject is the same, e.g., in terms of weight or number of molecules, as the amount administered free. In some embodiments, the Cₘₐₓ is measured by the presence of free labeled therapeutic peptide in the plasma. In some embodiments, the Cₘₐₓ measurement(s) are taken over a time period of 30 minutes, 1 hour, 2 hours, 3 hours, 6 hours, 12 hours, 24 hours, 2 days, or 7 days. In some embodiments, the time period begins at the time of, or 1 minute, 10 minutes, 60 minutes, 2 hours, 12 hours 24 hours, 2 days or 7 days after, administration of a dose of the composition or therapeutic peptide. In some embodiments, the subject is any of a mouse, rat, dog, or human.

In some embodiments, the composition, when administered to a subject, results in a volume of distribution (V_{z}) that is less than 90, 80, 70, 60, 50, 40, 30, 20, 10, 5, or 1% of that the V_{z} of the therapeutic peptide administered free to the subject.

In some aspects, the disclosure features a single dosage unit comprising a plurality of CDP-therapeutic peptide conjugates described herein or a composition described herein.

In some aspects, the disclosure features a method of treating a subject having a disorder comprising administering to said subject an effective amount of particles described herein or a composition described herein.

In some aspects, the disclosure features a CDP-therapeutic peptide conjugate comprising a therapeutic peptide covalently attached to a cyclodextrin-containing polymer (CDP), e.g., the therapeutic peptide is covalently attached to the CDP via the carboxy terminal, the therapeutic peptide is covalently attached to the CDP via the amino terminal and/or the therapeutic peptide is covalently attached to the CDP via an amino acid side chain.

In some embodiments, single therapeutic peptide is covalently attached to a CDP. In other embodiments, a plurality of therapeutic peptides are covalently attached to a single CDP.

In some aspects, the disclosure features a therapeutic peptide-CDP conjugate made by a method described herein.

In some instances, a protein can be used instead of a therapeutic peptide in any of the aspects and embodiments described above. A “protein”, as used herein, has more than 100 amino acids or more, e.g., the protein is at least 110 amino acids in length.

The details of one or more embodiments of the invention are set forth in the description below. Other features, objects, and advantages of the invention will be apparent from the description and the claims.

### DETAILED DESCRIPTION OF THE INVENTION

The present invention relates to cyclodextrin containing polymers conjugated to a therapeutic peptide (e.g., 1, 2, 3, 4, 5, 10, 15, or more therapeutic peptides), (CDP-therapeutic peptide conjugates), compositions of CDP-therapeutic peptide conjugates, therapeutic delivery systems containing CDP-therapeutic peptide conjugates, dosage forms containing CDP-therapeutic peptide conjugates, mixtures containing cyclodextrin-containing polymers and CDP-therapeutic peptide conjugates, and methods of use thereof. In certain embodiments, attachment of a therapeutic peptide to a cyclodextrin-containing polymer described herein can improve therapeutic peptide stability, therapeutic peptide solubility, reduce therapeutic peptide toxicity, and/or improve efficacy of the therapeutic peptide (for example, when used in vivo).

By selecting from a variety of linker groups used to link a therapeutic peptide to a CDP, the rate of therapeutic peptide release from the CDP can be attenuated for controlled delivery. The invention also relates to methods of treating subjects, e.g., humans, with a CDP-therapeutic peptide conjugate described herein.

CDP conjugates featured in the present invention may be useful to improve solubility and/or stability of a bioactive/therapeutic agent, such as therapeutic peptide, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into CDP conjugates of the invention.

### BRIEF DESCRIPTION OF THE DRAWINGS

FIG. 1 shows a calculated strand dependence on particle size. The CRLX101 particle size dependence on Conjugate number is depicted.

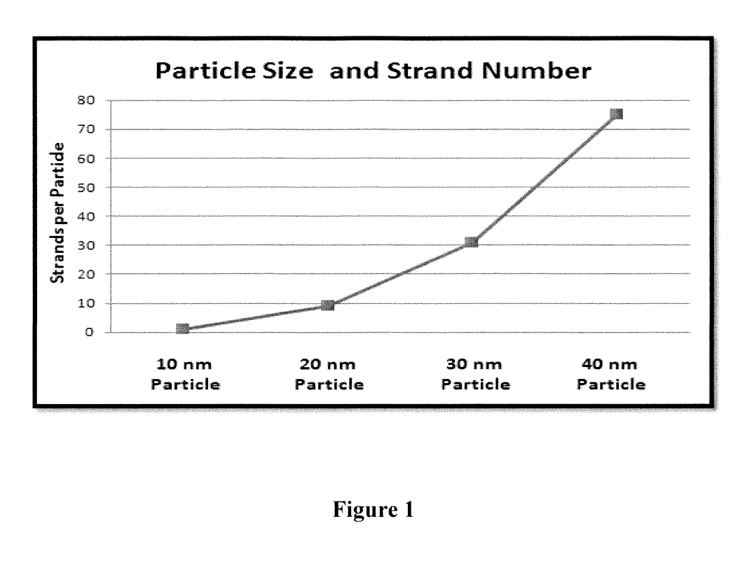

### DEFINITIONS

The term “ambient conditions,” as used herein, refers to surrounding conditions at about one atmosphere of pressure, 50% relative humidity and about 25° C.

The term “attach,” as used herein with respect to the relationship of a first moiety to a second moiety, e.g. the attachment of a therapeutic peptide to a polymer, refers to the formation of a covalent bond between a first moiety and a second moiety. In the same context, “attachment” refers to the covalent bond. For example, a therapeutic peptide attached to a polymer is a therapeutic peptide covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). E.g., where an attachment is through a linker, a first moiety (e.g., a drug) is covalently bonded to a linker, which in turn is covalently bonded to a second moiety (e.g., a hydrophobic polymer described herein).

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradation,” as used herein, encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

The term “carbohydrate,” as used herein refers to and encompasses monosaccharides, disaccharides, oligosaccharides and polysaccharides.

The phrase “cleavable under physiological conditions” refers to a bond having a half life of less than about 100 hours, when subjected to physiological conditions. For example, enzymatic degradation can occur over a period of less than about five years, one year, six months, three months, one month, fifteen days, five days, three days, or one day upon exposure to physiological conditions (e.g., an aqueous solution having a pH from about 4 to about 8, and a temperature from about 25° C. to about 37° C.).

An “effective amount” or “an amount effective” refers to an amount of the CDP-therapeutic peptide conjugate which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

The term “anionic moiety” refers to a moiety, which has a negative charge in at least one of the following conditions: during the production of a particle described herein, when formulated into a particle described herein, or subsequent to administration of a particle described herein to a subject, for example, while circulating in the subject and/or while in the endosome. Anionic moieties include polymeric species, such as moieties having more than one charge.

The term “anionic polymer” refers to an anionic moiety that has a plurality of negative charges (i.e., at least 2) when formulated into a particle described herein. In some embodiments, the anionic polymer has at least 3, 4, 5, 10, 15, or 20 negative charges.

The term “cationic moiety” refers to a moiety, which has a positive charge in at least one of the following conditions: during the production of a particle described herein, when formulated into a particle described herein, or subsequent to administration of a particle described herein to a subject, for example, while circulating in the subject and/or while in the endosome. Cationic moieties include polymeric species, such as moieties having more than one charge (e.g., a cationic PVA and/or a polyamine).

The term “cationic polymer” refers to a cationic moiety that has a plurality of positive charges (i.e., at least 2) when formulated into a particle described herein. In some embodiments, the cationic polymer has at least 3, 4, 5, 10, 15, or 20 positive charges.

The term “zwitterionic moiety” refers to a moiety, which has both a positive and a negative charge in at least one of the following conditions: during the production of a particle described herein, when formulated into a particle described herein, or subsequent to administration of a particle described herein to a subject, for example, while circulating in the subject and/or while in the endosome. Zwitterionic moieties include polymeric species, such as moieties having more than one charge.

“Pharmaceutically acceptable carrier or adjuvant,” as used herein, refers to a carrier or adjuvant that may be administered to a patient, together with a CDP-therapeutic peptide conjugate described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. In some cases, the polymer is biologically derived, i.e., a biopolymer. Non-limiting examples of biopolymers include peptides or proteins (i.e., polymers of various amino acids), or nucleic acids such as DNA or RNA. In some instances, a polymer may be comprised of subunits, e.g., a subunit described herein, wherein a subunit comprises polymers, e.g., PEG, but the subunit may be repeated within a conjugate. In some embodiments, a conjugate may comprise only one subunit described herein; however conjugates may comprise more than one identical subunit.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

A “hydroxy protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, acyl (e.g., acetyl), triethylsilyl (TES), t-butyldimethylsilyl (TBDMS), 2,2,2-trichloroethoxycarbonyl (Troc), and carbobenzyloxy (Cbz).

“Inert atmosphere,” as used herein, refers to an atmosphere composed primarily of an inert gas, which does not chemically react with the CDP-therapeutic peptide conjugates, particles, compositions or mixtures described herein. Examples of inert gases are nitrogen (N₂), helium, and argon.

“Linker,” as used herein, is a moiety having at least two functional groups. One functional group is capable of reacting with a functional group on a polymer described herein, and a second functional group is capable of reacting with a functional group on agent described herein. In some embodiments the linker has just two functional groups. A linker may have more than two functional groups (e.g., 3, 4, 5, 6, 7, 8, 9, 10 or more functional groups), which may be used, e.g., to link multiple agents to a polymer. Depending on the context, linker can refer to a linker moiety before attachment to either of a first or second moiety (e.g., agent or polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. It can be used to protect nanoparticles, liposomes, and/or micelles during lyophilization, for example to reduce or prevent aggregation, particle collapse and/or other types of damage. In an embodiment the lyoprotectant is a cryoprotectant. In an embodiment the lyoprotectant is a carbohydrate.

As used herein, the term “prevent” or “preventing” as used in the context of the administration of an agent to a subject, refers to subjecting the subject to a regimen, e.g., the administration of a CDP-therapeutic peptide conjugate such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The term “therapeutic peptide,” as used herein, refers to a peptide comprising two or more amino acids but not more than 100 amino acids, covalently linked together through one or more amide bonds, wherein upon administration of the peptide to a subject, the subject receives a therapeutic effect (e.g., administration of the therapeutic peptide treats a cell, or cures, alleviates, relieves or improves a symptom of a disorder) as opposed to, e.g., the use of a peptide as a linker which itself has no therapeutic effect. A therapeutic peptide may comprise, e.g., more than two, three, four, five, six, seven, eight, nine, ten, eleven, twelve, thirteen, fourteen, fifteen amino acids. In some embodiments, a therapeutic peptide comprises more than 15, e.g., greater than 20, 25, 30, 35, 40, 45, 50, 55, 60, 65, 70, 75, 80, 85, or 90 amino acids. For example, in some embodiments, the therapeutic peptide is more than 9, 10, 11 or 12 amino acids in length.

The therapeutic effect of the therapeutic peptide can occur by the therapeutic peptide acting as an agonist or as an antagonist. The term “agonist,” as used herein, is meant to refer to a peptide that mimics, or up-regulates, (e.g., potentiates or supplements) the activity of a protein. A direct agonist has at least one activity of the species to be agonized. E.g., a direct agonist can be a wild-type peptide or derivative thereof that has at least one activity of the wild-type protein. An indirect agonist can be a peptide which increases at least one activity of a protein. An indirect agonist includes a peptide which increases the interaction of a polypeptide with another molecule, e.g., a target peptide or nucleic acid. “Antagonist” as used herein is meant to refer to a peptide that reduces or down regulates (e.g., suppresses or inhibits) at least one activity of a protein. A direct antagonist can be a peptide which inhibits or decreases the interaction between a protein and another molecule, e.g., a target peptide or enzyme substrate. An indirect antagonist can be a peptide which reduces the amount of expressed protein present. In some embodiments, the therapeutic peptide is an agonist or an antagonist of a cytokine, a protease, a kinase or a membrane protein.

Exemplary therapeutic peptides include, e.g., a peptide that treats a cell, or cures, alleviates, relieves or improves a symptom of a metabolic disorder, e.g., a hormone, e.g., an anti-diabetogenic peptide; a peptide that treats a cell, or cures, alleviates, relieves or improves a symptom of a proliferative disorder, e.g., a tumor or metastases thereof; a peptide that treats a cell, or cures, alleviates, relieves or improves a symptom of a cardiovascular disorder; a peptide that treats a cell, or cures, alleviates, relieves or improves a symptom of an infectious disease; and a peptide that treats a cell, or cures, alleviates, relieves or improves a symptom of an allergic, inflammatory or autoimmune disorder. In some instances, the therapeutic peptide is not a hormone. For example, in some embodiments, the therapeutic peptide is a peptide other than luteinizing hormone releasing hormone (LHRH). In some embodiments, the therapeutic peptide is a peptide other than tubulysin. In some embodiments, the therapeutic peptide does not interact with, e.g., bind to an integrin. For example, in one embodiment, the therapeutic peptide does not have the sequence Arg-Gly-Asp.

Therapeutic peptides can comprise α-, β- and/or γ-amino acids. For example, the therapeutic peptide can comprise three or more α-amino acids, e.g., three or more consecutive α-amino acids. In one embodiment, the therapeutic peptide comprises at least four, five, six, seven, eight, nine, ten, or more α-amino acids, e.g., at least four, five, six, seven, eight, nine, ten, or more consecutive α-amino acids. Typically, all of the amino acids of the therapeutic peptide are α-amino acids or the therapeutic peptide includes less than 5, 4, 3 or 2 non-α amino acids. A therapeutic peptide may be linear, branched, cyclic, or a combination thereof.

In some instances, the therapeutic peptide is a “standard therapeutic peptide”, i.e., the majority of the amino acids (i.e., greater than 50% of the amino acids, e.g., 51%, 55%, 60%, 70%, 80%, 85%, 90%, 95%, 99%, or all of the amino acids) of the therapeutic peptide are standard amino acids. Standard amino acids are Ala, Arg, Asn, Asp, Cys, Gln, Glu, Gly, His, Ile, Leu, Lys, Met, Phe, Pro, Ser, Thr, Trp, Tyr, Val, Asx, and Glx. In other embodiments, the therapeutic peptide is a “non-standard therapeutic peptide”, i.e., the majority of the amino acids (i.e., greater than 50% of the amino acids, e.g., 51%, 55%, 60%, 70%, 80%, 85%, 90%, 95%, 99%, or all of the amino acids) of the therapeutic peptide are non-standard amino acids. The term “non-standard amino acid”, as used herein, refers to amino acids that have the required amino group, carboxylic acid, and side chain, but are not Ala, Arg, Asn, Asp, Cys, Gln, Glu, Gly, His, Ile, Leu, Lys, Met, Phe, Pro, Ser, Thr, Trp, Tyr, Val, Asx, or Glx.

The “therapeutic peptide” can be a fragment of a protein, e.g., a fragment having an amino acid sequence corresponding to the sequence of a commercially-available protein. In some embodiments, the therapeutic peptide is a fragment having an amino acid sequence corresponding to the sequence of a commercially available reference protein, and the glycan structure of the fragment differs from the glycan structure of the fragment from the commercially-available protein fragment. For example, the glycan structure of the therapeutic peptide may differ from the naturally-occurring glycosylation pattern of the peptide by one or more glycans, e.g., two, e.g., three, e.g., four, e.g., five, e.g., six, e.g., seven, e.g., eight, e.g., nine, e.g., ten or greater glycans.

In preferred embodiments, the therapeutic peptide is attached to the polymer via a linker (e.g., through a covalently linked chain of one or more atoms disposed between the therapeutic peptide and the polymer. The linker can be, e.g., a linker described herein.

In an embodiment, the therapeutic peptide has no substantial effect on the localization of the particle, e.g., it does not target the particle by affinity to a ligand, e.g., a surface protein or extracellular matrix component.

In some embodiments, if the conjugate includes a targeting agent that is a peptide, the targeting agent is a peptide that differs from the therapeutic peptide.

As used herein, the term “treat” or “treating” a subject having a disorder refers to subjecting the subject to a regimen, e.g., the administration of a CDP-therapeutic peptide conjugate such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl (CH₃C(O)—), benzoyl (C₆H₅C(O)—), and acetylamino acids (e.g., acetylglycine, CH₃C(O)NHCH₂C(O)—.

The term “alkoxy” refers to an alkyl group, as defined below, having an oxygen radical attached thereto. Representative alkoxy groups include methoxy, ethoxy, propyloxy, tert-butoxy and the like.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure. The term “alkylenyl” refers to a divalent alkyl, e.g., —CH₂—, —CH₂CH₂—, and —CH₂CH₂CH₂—.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The term “carboxy” refers to a —C(O)OH or salt thereof.

The term “hydroxy” and “hydroxyl” are used interchangeably and refer to —OH.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

CDP-Therapeutic Peptide Conjugates

Described herein are cyclodextrin containing polymer (“CDP”)-therapeutic peptide conjugates, wherein one or more therapeutic peptides are covalently attached to the CDP (e.g., either directly or through a linker). The CDP-therapeutic peptide conjugates include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, and 6,884,789, as well as U.S. Publication Nos. 20040087024, 20040109888, 20070025952, 20080058427, and 20080176958.

Accordingly, in one embodiment the CDP-therapeutic peptide conjugate is represented by Formula I:

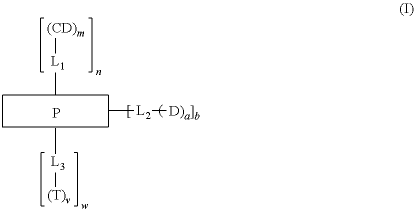

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a therapeutic peptide or a prodrug thereof;

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the therapeutic peptide moieties in the CDP-therapeutic peptide conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In certain embodiments, P contains a plurality of cyclodextrin moieties within the polymer chain as opposed to the cyclodextrin moieties being grafted on to pendant groups off of the polymeric chain. Thus in certain embodiments, the polymer chain of formula I further comprises n′ units of U, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U is represented by one of the general formulae below:

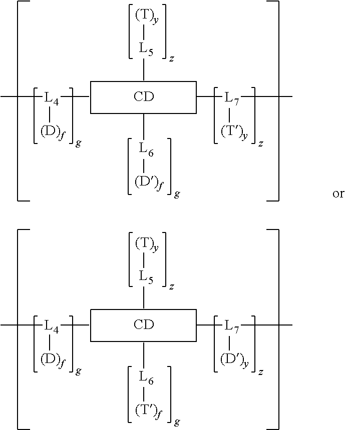

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different therapeutic peptide or prodrug forms thereof;

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

In some embodiments, both z moieties are 0. In some embodiments, one g is at least 1 and one g is 0 (e.g., the g in the polymer backbone chain is at least 1).

Preferably the polymer has a plurality of D or D′ moieties. In some embodiments, at least 50% of the U units have at least one D or D′. In some embodiments, one or more of the therapeutic peptide moieties in the CDP-therapeutic peptide conjugate can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include (e.g., comonomers of the CDP may include) polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA). N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-therapeutic peptide conjugate is represented by Formula II:

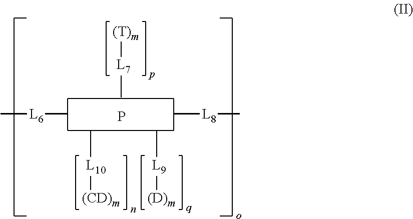

wherein

P represents a monomer unit of a polymer that comprises cyclodextrin moieties;

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a therapeutic peptide or a prodrug form thereof;

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the therapeutic peptide moieties in the CDP-therapeutic peptide conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-therapeutic peptide conjugate is represented either of the formulae below:

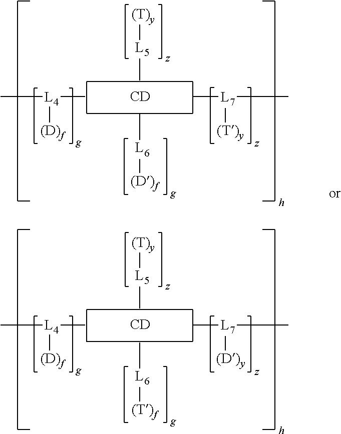

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different therapeutic peptide or prodrug thereof;

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

In some embodiments, both z moieties are 0. In some embodiments, one g is at least 1 and one g is 0 (e.g., the g in the polymer backbone chain is at least 1).

Preferably the polymer has a plurality of D or D′ moieties. In some embodiments, at least 50% of the polymer repeating units have at least one D or D′. In some embodiments, one or more of the therapeutic peptide moieties in the CDP-therapeutic peptide conjugate can be replaced with another therapeutic agent, e.g., another anticancer agent or anti-inflammatory agent.

In preferred embodiments, L4 and L7 represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6, 15-crown-5, 12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Exemplary cyclic moieties include cyclodextrins such as alpha, beta or gamma cyclodextrins. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL.

Thus, in certain embodiments, the invention relates to novel compositions of therapeutic cyclodextrin-containing polymeric compounds designed for drug delivery of a therapeutic peptide. In certain embodiments, these CDPs improve drug stability and/or solubility, and/or reduce toxicity, and/or improve efficacy of the therapeutic peptide when used in vivo. Furthermore, by selecting from a variety of linker groups, and/or targeting ligands, the rate of therapeutic peptide release from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

Described herein are CDP-therapeutic peptide conjugates, wherein one or more therapeutic peptide is covalently attached to the CDP. The CDP can include linear or branched cyclodextrin-containing polymers and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, 20080058427, and 20080176958, which are incorporated herein by reference in their entirety.

In some embodiments, the CDP-therapeutic peptide conjugate comprises a water soluble linear polymer conjugate comprising: cyclodextrin moieties; comonomers which do not contain cyclodextrin moieties (comonomers); and a plurality of therapeutic peptides; wherein the CDP-therapeutic peptide conjugate comprises at least four, five six, seven, eight, etc., cyclodextrin moieties and at least four, five six, seven, eight, or more, comonomers. In some embodiments, the therapeutic peptide is a therapeutic peptide described herein. The therapeutic peptide can be attached to the CDP via a functional group such as a hydroxyl group, or where appropriate, an amino group.

In some embodiments, one or more of the therapeutic peptide moieties in the CDP-therapeutic peptide conjugate can be replaced with another therapeutic agent, e.g., another anticancer agent, or another anti-inflammatory agent, or another cardiac agent, or another nephrology agent.

In some embodiments, at least four cyclodextrin moieties and at least four comonomers alternate in the CDP-therapeutic peptide conjugate. In some embodiments, said therapeutic peptides are cleaved from said CDP-therapeutic peptide conjugate under biological conditions to release therapeutic peptide. In some embodiments, the cyclodextrin moieties comprise linkers to which therapeutic peptides are linked. In some embodiments, the therapeutic peptides are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic peptide was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring. In some embodiments, the cyclodextrin moiety comprises an alpha, beta, or gamma cyclodextrin moiety. In some embodiments, at least about 50% of available positions on the CDP are reacted with a therapeutic peptide and/or a linker therapeutic peptide (e.g., at least about 55%, 60%, 65%, 70%, 75%, 80%, 85%, 90%, or 95%). In some embodiments, the therapeutic peptide is at least 5%, 10%, 15%, 20%, 25%, 30%, or 35% by weight of CDP-therapeutic peptide conjugate.

In some embodiments, the therapeutic peptide is poorly soluble in water. In some embodiments, the solubility of the therapeutic peptide is <5 mg/ml at physiological pH. In some embodiments, the therapeutic peptide is a hydrophobic compound with a log P>0.4, >0.6, >0.8, >1, >2, >3, >4, or >5.

In some embodiments, a therapeutic delivery system comprises a CDP-therapeutic peptide conjugate and one or more surfactants. Optionally, the surfactant may be PEG, poly(vinyl alcohol) (PVA), poly(vinylpyrrolidone) (PVP), poloxamer, a polysorbate, a polyoxyethylene ester, a PEG-lipid (e.g., PEG-ceramide, d-alpha-tocopheryl polyethylene glycol 1000 succinate), 1,2-distearoyl-sn-glycero-3-phosphoethanolamine or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is SOLUTOL® HS 15 (BASF, Florham Park, N.J.). In some embodiments, the surfactant may be present in an amount of up to about 35% by weight of the therapeutic delivery system (e.g., up to about 20% by weight or up to about 25% by weight, from about 15% to about 35% by weight, from about 20% to about 30% by weight, or from about 23% to about 26% by weight).

In some embodiments, the therapeutic delivery system further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

A therapeutic delivery system described herein may also include one or more counter ions, e.g., a charge moiety, a cationic moiety, an anionic moiety, or a zwitterionic moiety. The counter ion may neutralize a charge associated with a therapeutic peptide thereby allowing for improved formulations (e.g., improved stability, solubility, or transport). In some embodiments, the charged moiety is associated with a therapeutic peptide (e.g., hydrogen bonded to the therapeutic peptide, or part of a solvation layer around the therapeutic peptide). In some embodiments, the charged moiety is covalently attached to a polymer of the delivery therapeutic delivery system. In some embodiments, the charged moiety is covalently attached to a polymer that is covalently attached to a therapeutic peptide. In some embodiments the charged moiety is another peptide.

In some embodiments, a charged moiety is covalently attached to a CDP via a linker (e.g., at the carboxy terminal or hydroxyl terminal of the hydrophobic polymers). In some embodiments, the linker comprises a bond formed using click chemistry (e.g., as described in WO 2006/115547). In some embodiments, the linker comprises an amide bond, an ester bond, a disulfide bond, a sulfide bond, a ketal, a succinate, or a triazole. In some embodiments, a single charged moiety is covalently attached to a single CDP. In some embodiments, a charged moiety is covalently attached to a CDP via an amide, ester or ether bond. In some embodiments, a CDP is covalently attached to a plurality of charged moieties.

In some embodiments, a cationic moiety is a cationic polymer (e.g., PEI, cationic PVA, poly(histidine), poly(lysine), or poly(2-dmethylamino)ethyl methacrylate). In some embodiments, a cationic moiety is an amine (e.g. a primary, secondary, tertiary or quaternary amine). In some embodiments, at least a portion of the cationic moieties comprise a plurality of amines (e.g., a primary, secondary, tertiary or quaternary amines). In some embodiments, at least one amine in the cationic moiety is a secondary or tertiary amine. In some embodiments, at least a portion of the cationic moieties comprise a polymer, for example, polyethylene imine or polylysine Polymeric cationic moieties have a variety of molecular weights (e.g., ranging from about 500 to about 5000 Da, for example, from about 1 to about 2 kDa or about 2.5 kDa).

In some embodiments the cationic moiety is a polymer, for example, having one or more secondary or tertiary amines, for example cationic PVA (e.g., as provided by Kuraray, such as CM-318 or C-506), chitosan, and polyethyleneamine. Cationic PVA can be made, for example, by polymerizing a vinyl acetate/N-vinylformamide co-polymer, e.g., as described in US 2002/0189774, the contents of which are incorporated herein by reference. Other examples of cationic PVA include those described in U.S. Pat. No. 6,368,456 and Fatehi (Carbohydrate Polymers 79 (2010) 423-428, the contents of which are incorporated herein by reference. In some embodiments, at least a portion of the cationic moieties of comprise a cationic PVA (e.g., as provided by Kuraray, such as CM-318 or C-506).

Other exemplary cationic moieties include poly(histidine) and poly(2-dmethylamino)ethyl methacrylate). In some embodiments, the amine is positively charged at acidic pH. In some embodiments, the amine is positively charged at physiological pH. In some embodiments, at least a portion of the cationic moieties are selected from the group consisting of protamine sulfate, hexademethrine bromide, cetyl trimethylammonium bromide, spermine, and spermidine. In some embodiments, at least a portion of the cationic moieties are selected from the group consisting of tetraalkyl ammonium moieties, trialkyl ammonium moieties, imidazolium moieties, aryl ammonium moieties, iminium moieties, amidinium moieties, guanadinium moieties, thiazolium moieties, pyrazolylium moieties, pyrazinium moieties, pyridinium moieties, and phosphonium moieties. In some embodiments, at least a portion of the cationic moieties are cationic lipids. In some embodiments, at least a portion of the cationic moieties are conjugated to a non-polymeric hydrophobic moiety (e.g., cholesterol or Vitamin E TPGS). In some embodiments, the plurality of cationic moieties are from about 1 to about 60 weight % of the particle. In some embodiments, the ratio of the charge of the plurality of cationic moieties to the charge from the plurality of therapeutic peptides is from about 1:1 to about 50:1 (e.g., 1:1 to about 10:1 or 1:1 to 5:1).

Exemplary cationic moieties for use in the particles and conjugates described herein include amines such as polyamines (e.g., polyethyleneimine (PEI) or derivatives thereof such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives), cationic lipids (e.g. DOTIM, dimethyldioctadecyl ammonium bromide, 1,2 dioleyloxypropyl-3-trimethyl ammonium bromide, DOTAP, 1,2-dimyristyloxypropyl-3-dimethyl-hydroxyethyl ammonium bromide, EDMPC, ethyl-PC, DODAP, DC-cholesterol, and MBOP, CLinDMA, pCLinDMA, eCLinDMA, DMOBA, and DMLBA), polyamino acids (e.g., poly(lysine), poly(histidine), and poly(arginine)) and polyvinyl pyrrolidone (PVP). The cationic moiety can be positively charged at physiological pH.

Additional exemplary cationic moieties include protamine sulfate, hexademethrine bromide, cetyl trimethylammonium bromide, spermine, spermidine, and those described for example in WO2005007854, U.S. Pat. No. 7,641,915, and WO2009055445, the contents of each of which are incorporated herein by reference. Cationic moieties may include N-methyl D-glucamine, choline, arginine, lysine, procaine, tromethamine (TRIS), spermine, N-methyl-morpholine, glucosamine, N,N-bis 2-hydroxyethyl glycine, diazabicycloundecene, creatine, arginine ethyl ester, amantadine, rimantadine, ornithine, taurine, and citrulline. Cationic moieties may additionally include sodium, potassium, calcium, magnesium, ammonium, monoethanolamine, diethanolamine, triethanolamine, tromethamine, lysine, histidine, arginine, morpholine, methylglucamine, and glucosamine.

Anionic moieties which may be suitable for formulation with net positively charged therapeutic peptides include, but are not limited to, acetate, propionate, butyrate, pentanoate, hexanoate, heptanoate, levulinate, chloride, bromide, iodide, citrate, succinate, maleate, glycolate gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, sulfonate, acetic acid, adamantoic acid, alpha keto glutaric acid, D- or L-aspartic acid, benzensulfonic acid, benzoic acid, 10-camphorsulfunic acid, citric acid, 1,2-ethanedisulfonic acid, fumaric acid, D-gluconic acid, D-glucuronic acid, glucaric acid. D- or L-glutamic acid, glutaric acid, glycolic acid, hippuric acid, hydrobromic acid, hydrochloric acid, 1-hydroxyl-2-napthoic acid, lactobioinic acid, maleic acid, L-malic acid, mandelic acid, methanesulfonic acid, mucic acid, 1,5 napthalenedisulfonic acid tetrahydrate, 2-napthalenesulfonic acid, nitric acid, oleic acid, pamoic acid, phosphoric acid, p-toluenesulfonic acid hydrate, D-saccharid acid monopotassium salt, salicyclic acid, stearic acid, succinic acid, sulfuric acid, tannic acid, D- or L-tartaric acid.

In some embodiments, pharmaceutical salts are formed by the inclusion of counter ions (e.g., charged moieties described herein) with particles or conjugates described herein.

In some embodiments, the therapeutic peptide is attached to the CDP via a second compound.

In some embodiments, administration of the CDP-therapeutic peptide conjugate to a subject results in release of the therapeutic peptide over a period of at least 6 hours. In some embodiments, administration of the CDP-therapeutic peptide conjugate to a subject results in release of the therapeutic peptide over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-therapeutic peptide conjugate to a subject the rate of therapeutic peptide release is dependent primarily upon the rate of hydrolysis as opposed to enzymatic cleavage.

In some embodiments, the CDP-therapeutic peptide conjugate has a molecular weight of 10,000-500,000. In some embodiments, the cyclodextrin moieties make up at least about 2%, 5%, 10%, 20%, 30%, 50% or 80% of the CDP-therapeutic peptide conjugate by weight.

In some embodiments, the CDP-therapeutic peptide conjugate is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-therapeutic peptide conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-therapeutic peptide conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP-therapeutic peptide conjugate is a polymer having attached thereto a plurality of D moieties of the following formula:

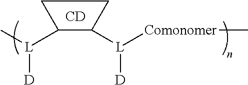

wherein each L is independently a linker, and each D is independently a therapeutic peptide, a prodrug derivative thereof, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one therapeutic peptide and in some embodiments, at least two therapeutic peptide moieties. In some embodiments, the molecular weight of the comonomer is from about 2000 to about 5000 Da (e.g., from about 2000 to about 4500, from about 3000 to about 4000 Da, or less than about 4000, (e.g., about 3400 Da)).

In some embodiments, the therapeutic peptide is a therapeutic peptide described herein. The therapeutic peptide can be attached to the CDP via a functional group such as a hydroxyl group, or where appropriate, an amino group. In some embodiments, one or more of the therapeutic peptide moieties in the CDP-therapeutic peptide conjugate can be replaced with another therapeutic agent, e.g., another anticancer agent or anti-inflammatory agent.

In some embodiments, the CDP-therapeutic peptide conjugate is a polymer having attached thereto a plurality of D moieties of the following formula:

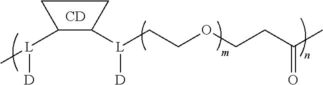

wherein each L is independently a linker, and each D is independently a therapeutic peptide, a prodrug derivative thereof, or absent, provided that the polymer comprises at least one therapeutic peptide and in some embodiments, at least two therapeutic peptide moieties (e.g., at least 3, 4, 5, 6, 7, 8, 9, 10, 11, 12, 13, 14, 15, 16, 17, 18, 19, 20 or more); and wherein the group

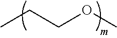

has a Mw of 4.0 kDa or less, e.g., 3.2 to 3.8 kDa, e.g., 3.4 kDa and n is at least 4, 5, 6, 7, 8, 9, 10, 11, 12, 13, 14, 15, 16, 17, 18, 19 or 20.

In some embodiments, the therapeutic peptide is a therapeutic peptide described herein. The therapeutic peptide can be attached to the CDP via a functional group such as a hydroxyl group, or where appropriate, an amino group. In some embodiments, one or more of the therapeutic peptide moieties in the CDP-therapeutic peptide conjugate can be replaced with another therapeutic agent, e.g., an anticancer agent or anti-inflammatory agent.

In some embodiments, less than all of the L moieties are attached to D moieties, meaning in some embodiments, at least one D is absent. In some embodiments, the loading of the D moieties on the CDP-therapeutic peptide conjugate is from about 1 to about 50% (e.g., from about 1 to about 25%, from about 5 to about 20% or from about 5 to about 15%). In some embodiments, each L independently comprises an amino acid or a derivative thereof. In some embodiments, each L independently comprises a plurality of amino acids or derivatives thereof. In some embodiments, each L is independently a dipeptide or derivative thereof.

In some embodiments, the CDP-therapeutic peptide conjugate is a polymer having attached thereto a plurality of L-D moieties of the following formula:

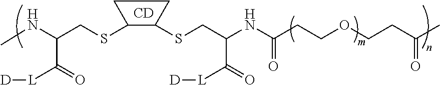

wherein each L is independently a linker or absent and each D is independently a therapeutic peptide, a prodrug derivative thereof, or absent and wherein the group

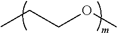

has a Mw of 5.0 kDa or less, e.g., 4.5 kDa or less, e.g., 4.0 kDa or less e.g., 3.2 to 3.8 kDa, e.g., 3.4 kDa and n is at least 4, 5, 6, 7, 8, 9, 10, 11, 12, 13, 14, 15, 16, 17, 18, 19 or 20, provided that the polymer comprises at least one therapeutic peptide and in some embodiments, at least two therapeutic peptide moieties (e.g., at least 3, 4, 5, 6, 7, 8, 9, 10, 11, 12, 13, 14, 15, 16, 17, 18, 19, 20 or more).

In some embodiments, the therapeutic peptide is a therapeutic peptide described herein.

In some embodiments, less than all of the C(═O) moieties are attached to L-D moieties, meaning in some embodiments, at least one L and/or D is absent. For example, in some embodiments, the CDP-therapeutic peptide conjugate is of the formula

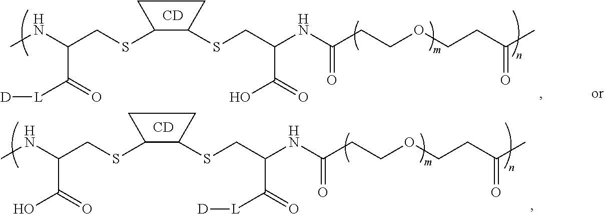

with the variables as defined above.

In some embodiments, the loading of the L, D and/or L-D moieties on the CDP-therapeutic peptide conjugate is from about 1 to about 50% (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the therapeutic peptide moieties in the CDP-therapeutic peptide conjugate can be replaced with another therapeutic agent, e.g., another anticancer agent or anti-inflammatory agent.

In some embodiments, the CDP-therapeutic peptide conjugate is a polymer having the following formula:

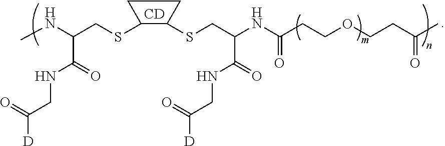

In some embodiments, less than all of the C(═O) moieties are attached to

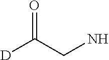

moieties, meaning in some embodiments,

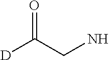

is absent, provided that the polymer comprises at least one therapeutic peptide and in some embodiments, at least two therapeutic peptide moieties (e.g., at least 3, 4, 5, 6, 7, 8, 9, 10, 11, 12, 13, 14, 15, 16, 17, 18, 19, 20 or more). In some embodiments, the loading of the

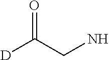

moieties on the CDP-therapeutic peptide conjugate is from about 1 to about 50% (e.g., from about 1 to about 25%, from about 5 to about 25% or from about 15 to about 15%).

In some embodiments, the therapeutic peptide is a therapeutic peptide described herein.

In some embodiments, one or more of the therapeutic peptide moieties in the CDP-therapeutic peptide conjugate can be replaced with another therapeutic agent, e.g., another anticancer agent or anti-inflammatory agent.

In some embodiments, the CDP-therapeutic peptide conjugate will contain a therapeutic peptide and at least one additional therapeutic agent. For instance, a therapeutic peptide and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

Cyclodextrins

In certain embodiments, the cyclodextrin moieties make up at least about 2%, 5% or 10% by weight, up to 20%, 30%, 50% or even 80% of the CDP by weight. In certain embodiments, the therapeutic peptides, or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 Daltons, preferably from about 5000 to about 200,000 Daltons and, even more preferably, from about 10,000 to about 100,000. Most preferably, Mₙ varies between about 12,000 and 65,000 Daltons. In certain other embodiments, Mₙ varies between about 3000 and 150,000 Daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

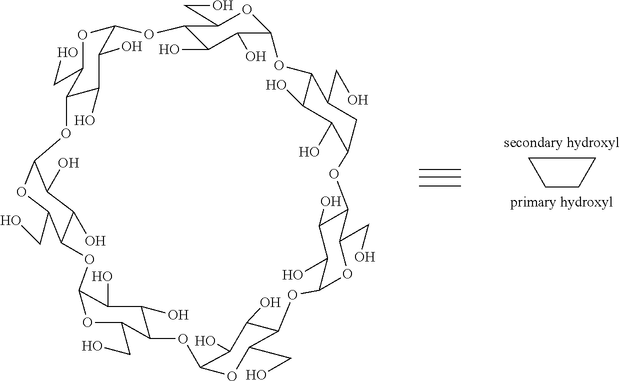

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-therapeutic peptide conjugate is oxidized. In certain embodiments, the cyclodextrin moieties of P alternate with linker moieties in the polymer chain.

### Comonomers

In addition to a cyclodextrin moiety, the CDP can also include a comonomer, for example, a comonomer described herein. In some embodiments, a comonomer of the CDP-therapeutic peptide conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-therapeutic peptide conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, a comonomer can be and/or can comprise a linker such as a linker described herein.

### Linkers/Tethers

The CDPs described herein can include one or more linkers. In some embodiments, a linker, such as a linker described herein, can link a cyclodextrin moiety to a comonomer. In some embodiments, a linker can link a therapeutic peptide to a CDP. In some embodiments, for example, when referring to a linker that links a therapeutic peptide to the CDP, the linker can be referred to as a tether.

In certain embodiments, a plurality of the linker moieties is attached to a therapeutic peptide or prodrug thereof and are cleaved under biological conditions.

Described herein are CDP-therapeutic peptide conjugates that comprise a CDP covalently attached to therapeutic peptides through attachments that are cleaved under biological conditions to release the therapeutic peptide. In certain embodiments, a CDP-therapeutic peptide conjugate comprises a therapeutic peptide covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the therapeutic peptide.

In some embodiments, such therapeutic peptides are covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-therapeutic peptide conjugate comprises a therapeutic peptide covalently attached to the CDP through a tether, wherein the tether comprises a self-cyclizing moiety. In some embodiments, the tether further comprises a selectivity-determining moiety. Thus, one aspect of the invention relates to a polymer conjugate comprising a therapeutic agent covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

The linker may be, for example, an alkylenyl (divalent alkyl) group. In some embodiments, one or more carbon atoms of the alkylenyl linker may be replaced with one or more heteroatoms. In some embodiments, one or more carbon atoms may be substituted with a substituent (e.g., alkyl, amino, or oxo substituents).

In some embodiments, the linker, prior to attachment to the therapeutic peptide and the CDP, may have one or more of the following functional groups: amine, amide, hydroxyl, carboxylic acid, ester, halogen, thiol, maleimide, carbonate, or carbamate.

In some embodiments, the linker may comprise an amino acid linker or a peptide linker. Frequently, in such embodiments, the peptide linker is cleavable by hydrolysis, under reducing conditions, or by a specific enzyme.

When the linker is the residue of a divalent organic molecule, the cleavage of the linker may be either within the linker itself, or it may be at one of the bonds that couples the linker to the remainder of the conjugate, i.e. either to the agent or the polymer. Exemplary functional groups that can be part of the linker include esters, ethers, amides, disulfides, and thioethers. A linker may include a bond resulting from click chemistry (e.g., an amide bond, an ester bond, a ketal, a succinate, or a triazole and those described in WO 2006/115547).

In some embodiments, a linker may be selected from one of the following:

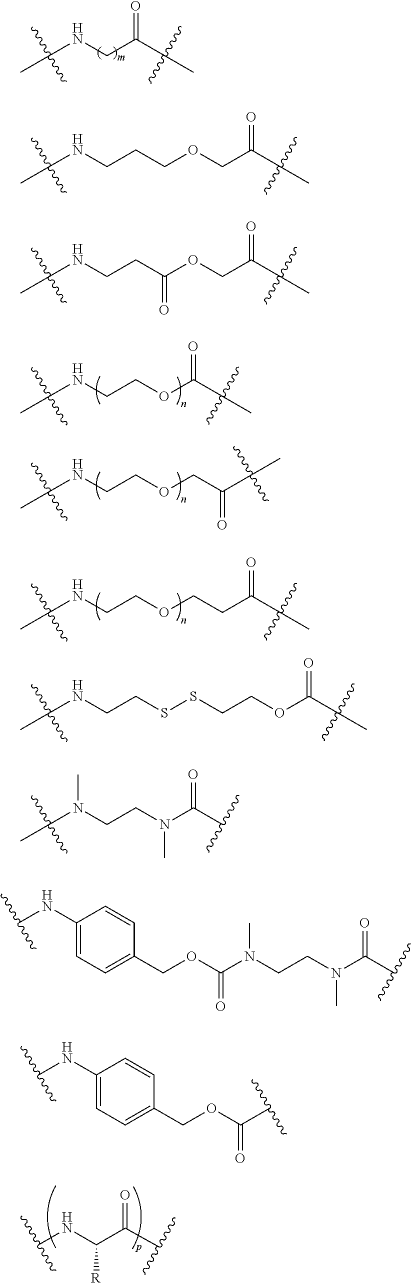

wherein m is 1-10, n is 1-10, p is 1-10, and R is an amino acid side chain.

A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids. In certain embodiments, the invention contemplates any combination of the foregoing. Those skilled in the art will recognize that, for example, any CDP of the invention in combination with any linker (e.g., a linker described herein such as a self-cyclizing moiety, any selectivity-determining moiety, and/or any therapeutic peptide) are within the scope of the invention.

In certain embodiments, the selectivity-determining moiety is selected such that the bond is cleaved under acidic conditions.

In certain embodiments where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

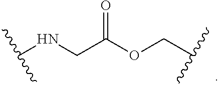 [SMILES: *COC(=O)CN*]

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA (SEQ ID NO:1) and GFLG (SEQ ID NO:2), preferably GFLG (SEQ ID NO:2).

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by Formula A:

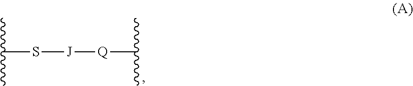

wherein S is a sulfur atom that is part of a disulfide bond; J is optionally substituted hydrocarbyl; and Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

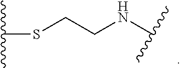 [SMILES: *NCCS*]

In certain embodiments, the selectivity-determining moiety is represented by Formula B:

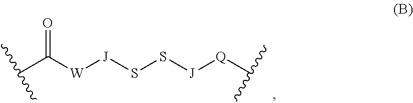

wherein W is either a direct bond or selected from lower alkyl, NR¹⁴, S, O;
S is sulfur; J, independently and for each occurrence, is hydrocarbyl or polyethylene glycol; Q is O or NR¹³, wherein R¹³ is hydrogen or alkyl; and R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂), —SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

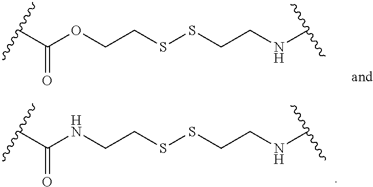 [SMILES: *NCCSSCCOC(*)=O ; *NCCSSCCNC(*)=O]

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

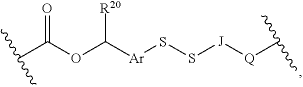

wherein Ar is a substituted or unsubstituted benzo ring; J is optionally substituted hydrocarbyl; and Q is O or NR¹³, wherein R¹³ is hydrogen or alkyl.

In certain embodiments, Ar is unsubstituted. In certain embodiments, Ar is a 1,2-benzo ring. For example, suitable moieties within Formula B include

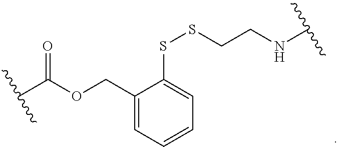 [SMILES: *NCCSSc1ccccc1COC(*)=O]

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone.

In certain embodiments, the self-cyclizing moiety has a structure

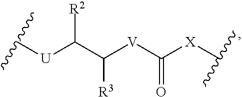

wherein U is selected from NR¹ and S; X is selected from O, NR⁵, and S, preferably O or S; V is selected from O, S and NR⁴, preferably O or NR⁴; R² and R³ are independently selected from hydrogen, alkyl, and alkoxy; or R² and R³ together with the carbon atoms to which they are attached form a ring; and R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl. In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together are —(CH₂)ₙ— wherein n is 3 or 4, thereby forming a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments, U is bonded to the self-cyclizing moiety.

In certain embodiments, the self-cyclizing moiety is selected from

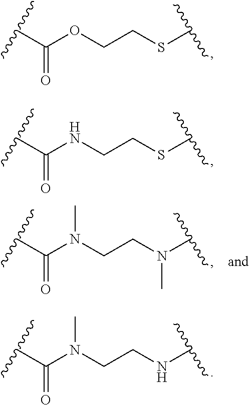

In certain embodiments, the selectivity-determining moiety may connect to the self-cyclizing moiety through carbonyl-heteroatom bonds, e.g., amide, carbamate, carbonate, ester, thioester, and urea bonds.

In certain embodiments, a therapeutic peptide is covalently attached to a polymer through a tether, wherein the tether comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH may be be a self-cyclizing moiety, and ABC may be selected such that enzyme Y cleaves between C and D. Once cleavage of the bond between C and D progresses to a certain point, D will cyclize onto H, thereby releasing therapeutic agent X, or a prodrug thereof.

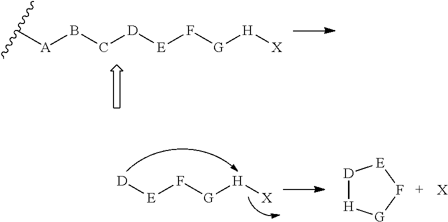

In certain embodiments, therapeutic peptide X may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker may be and/or comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, a linker group, e.g., between a therapeutic peptide and the CDP, comprises a self-cyclizing moiety. In certain embodiments, a linker group, e.g., between a therapeutic peptide and the CDP, comprises a selectivity-determining moiety.

In certain embodiments as disclosed herein, a linker group, e.g., between a therapeutic peptide and the CDP, comprises a self-cyclizing moiety and a selectivity-determining moiety.

In certain embodiments as disclosed herein, the therapeutic peptide or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to therapeutic peptides or prodrugs thereof that are cleaved under biological conditions.

In certain embodiments, at least one linker that connects the therapeutic peptide or prodrug thereof to the polymer comprises a group represented by the formula

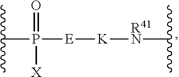

wherein P is phosphorus; O is oxygen; E represents oxygen or NR⁴⁰; K represents hydrocarbyl; X is selected from OR⁴² or NR⁴³R⁴⁴; and R⁴⁰, R⁴¹, R⁴², R⁴³, and R⁴⁴ independently represent hydrogen or optionally substituted alkyl.

In certain embodiments, E is NR⁴⁰ and R⁴⁰ is hydrogen.

In certain embodiments, K is lower alkylene (e.g., ethylene).

In certain embodiments, at least one linker comprises a group selected from

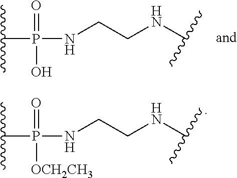

In certain embodiments, X is OR⁴².

In certain embodiments, the linker group comprises an amino acid or peptide, or derivative thereof (e.g., a glycine or cysteine).

In certain embodiments as disclosed herein, the linker is connected to the therapeutic peptide through a hydroxyl group (e.g., forming an ester bond). In certain embodiments as disclosed herein, the linker is connected to the therapeutic peptide through an amino group (e.g., forming an amide bond).

In certain embodiments, the linker group that connects to the therapeutic peptide may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

In one embodiment, the linker used to link therapeutic peptide to a CDP controls the rate of therapeutic peptide release from the CDP. For example, the linker can be a linker which in the PBS protocol described herein, releases within 24 hours as free therapeutic peptide, 70%, 75%, 80%, 85%, 86%, 87%, 88%, 89%, 90%, 91%, 92%, 93%, 94%, 95%, 96%, 97%, 98%, 99% or all of the therapeutic peptide in the CDP-conjugated therapeutic peptide initially present in the assay. In some embodiments, in the PBS protocol described herein, the linker releases 71±10% of the therapeutic peptide from the CDP-conjugated therapeutic peptide within 24 hours, wherein 71 is the % of therapeutic peptide released from the CDP-conjugated therapeutic peptide at 24 hours by a reference structure, e.g., a therapeutic peptide coupled via 2-(2-(2-aminoethoxy)ethoxy)acetic acetate (i.e., aminoethoxyethoxy) to the same CDP in the PBS protocol described herein. In other embodiments, the linker releases 88±10% of the therapeutic peptide from the CDP-conjugated therapeutic peptide within 24 hours, wherein 88 is the % of therapeutic peptide released from the CDP-conjugated therapeutic peptide at 24 hours by a reference structure, e.g., therapeutic peptide coupled via glycine to the same CDP in the PBS protocol described herein or the linker releases 95±5% of the therapeutic peptide from the CDP-conjugated therapeutic peptide within 24 hours, wherein 95 is the % of therapeutic peptide released from the CDP-conjugated therapeutic peptide at 24 hours by a reference structure, e.g., therapeutic peptide, coupled via alanine glycolate to the same CDP in the PBS protocol described herein. Such linkers include linkers which are released by hydrolysis of an ester bond, which hydrolysis releases therapeutic peptide conjugated to CDP from CDP. In one embodiment, the linker is selected from glycine, alanine glycolate and 2-(2-(2-aminoethoxy)ethoxy)acetic acetate (i.e. aminoethoxyethoxy). In one embodiment, the linker used to link therapeutic peptide to a CDP attaches to the therapeutic peptide via an ester linkage and the CDP via an amide linkage. In some preferred embodiments, the linker includes a heteroatom attached to the carbon positioned alpha to the carbonyl carbon that forms the ester linkage with the therapeutic peptide.

In one embodiment, the linker used to link therapeutic peptide to a CDP has the following formula

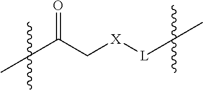

wherein X is O, NH, or Nalkyl; and L is an alkylenyl or heteroalkylenyl chain, wherein one or more of the carbons of the alkylenyl or heteroalkylenyl are optionally substituted (e.g., with an oxo moiety), or wherein L is absent; wherein the carbonyl portion of the linker attaches to the therapeutic peptide to form an ester linkage; and wherein the X-L portion of the linker attaches to the CDP to form an amide bond.

In one embodiment, X is NH. In one embodiment, X is NH and L is absent.

In one embodiment, X is O. In one embodiment, X is O and L is an alkylenyl or heteroalkylenyl chain, wherein one or more of the carbons of the alkylenyl or heteroalkylenyl are optionally substituted (e.g., with an oxo moiety). In one embodiment, L is —C(O)CH₂CH₂NH—.

In certain embodiments, the present invention contemplates a CDP, wherein a plurality of therapeutic peptides are covalently attached to the polymer through attachments that are cleaved under biological conditions to release the therapeutic agents as discussed above, wherein administration of the polymer to a subject results in release of the therapeutic agent over a period of at least 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month.

In some embodiments, the conjugation of the therapeutic peptide to the CDP improves the aqueous solubility of the therapeutic peptide and hence the bioavailability. Accordingly, in one embodiment of the invention, the therapeutic peptide has a log P>0.4, >0.6, >0.8, >1, >2, >4, or even >5.

The CDP-therapeutic peptide of the present invention preferably has a molecular weight in the range of 10,000 to 500,000; 30,000 to 200,000; or even 70,000 to 150,000 amu.

In certain embodiments, the present invention contemplates attenuating the rate of release of the therapeutic peptide by introducing various tether and/or linking groups between the therapeutic agent and the polymer. Thus, in certain embodiments, the CDP-therapeutic peptide conjugates of the present invention are compositions for controlled delivery of the therapeutic peptide.

### Therapeutic Peptides

Therapeutic peptides can be delivered to a subject using a CDP-therapeutic peptide conjugate, a therapeutic delivery system comprising a CDP-therapeutic peptide conjugate, particle or composition described herein. In some embodiments, the therapeutic peptide is a peptide with pharmaceutical activity. In another embodiment, the therapeutic peptide is a clinically used or investigated therapy. In another embodiment, the therapeutic peptide has been approved by the U.S. Food and Drug Administration for use in humans or other animals.

### Cancer

The disclosed CDP-therapeutic peptide conjugates are useful in treating proliferative disorders, e.g., treating a tumor and metastases thereof wherein the tumor or metastases thereof is a cancer described herein.

The therapeutic peptide can be, e.g., a peptide inhibitor of proliferative signaling (e.g., an inhibitor of mitogenic signaling or a peptide that restores the activity of a tumor suppressor protein such as p53), a cell cycle inhibitor, or an inducer of apoptosis. For example, a peptide inhibitor of proliferative signaling includes peptide inhibitors of Ras activation, peptide inhibitors of MAP kinase, a peptide inhibitor of NF-κB activation, and a peptide inhibitor of c-Myc activation. See, e.g., Bidwell et al. (2009) Expert Opin. Drug Delivery 6(10):1033-1047, the contents of which is incorporated herein by reference.

Examples of therapeutic peptides that can be used in the claimed conjugates, particles an compositions include the following:

A-6 (Angstrom Pharmaceuticals Inc.) an eight amino acid peptide, and variants and derivatives thereof, which can be used in the conjugates, therapeutic delivery systems, and compositions described herein to treat a proliferative disorder, e.g., cancer (e.g., ovarian cancer);

PPI-149 (abarelix, Plenaxis™), a ten amino acid peptide, and variants and derivatives thereof, which can be used in the conjugates, therapeutic delivery systems, and compositions described herein to treat a proliferative disorder such as cancer (e.g. prostate cancer);

ABT-510 (Abbott Laboratories), a nine amino acid peptide, and variants and derivatives thereof, which can be used in the conjugates, therapeutic delivery systems, and compositions described herein to treat a proliferative disorder such as cancer (e.g., lung cancer (e.g., small cell or non-small cell lung cancer), renal cell carcinoma, sarcoma, lymphoma, solid tumors, melanoma and malignant glioma);

ADH-1 (Exherin™, Adherex Technologies), a cyclic five amino acid peptide, and variants and derivatives thereof, which can be used in the conjugates, therapeutic delivery systems, and compositions described herein to treat a proliferative disorder such as cancer (e.g., solid tumors and melanoma);

AEZS-108 (AN-152, ZEN-008, AEtherna Zentaris), a ten amino acid peptide, and variants and derivatives thereof, which can be used in the conjugates, therapeutic delivery systems, and compositions described herein to treat proliferative disorders such as cancer (e.g., endometrial carcinoma, breast cancer, ovarian cancer, and prostate cancer);

afamelanotide (EP-1647, CUV-1647, Melanotan™, Clinuvel Pharmaceuticals, Ltd.) a thirteen amino acid peptide, and variants and derivatives thereof, which can be used in the conjugates, therapeutic delivery systems, and compositions described herein to treat a proliferative disorder such as cancer (e.g., skin cancer);

ambamustine (PTT-119, Abbott Laboratories) a three amino acid peptide, and variants and derivatives thereof, which can be used in the conjugates, therapeutic delivery systems, and compositions described herein to treat a proliferative disorder such as cancer (e.g., lymphoma (e.g., Non-Hodgkin lymphoma) and lung cancer (e.g., small cell or non-small cell lung cancer);

antagonist G (PTL-68001, Arana Therapeutics), a six amino acid peptide, and variants and derivatives thereof, which can be used in the conjugates, therapeutic delivery systems, and compositions described herein to treat a proliferative disorder such as cancer (e.g., lung cancer (e.g., small cell or non-small cell lung cancer), pancreatic cancer and colorectal cancer);

ATN-161 (Attenuon LLC), a five amino acid peptide, and variants and derivatives thereof, which can be used in the conjugates, therapeutic delivery systems, and compositions described herein to treat a proliferative disorder such as cancer (e.g., glioma);

avorelin (EP-23904, Meterelin™, Lutrelin™, Mediolanum Farmaceutici SpA), a nine amino acid peptide, and variants and derivatives thereon, which can be used in the conjugates, therapeutic delivery systems, and compositions described herein to treat a proliferative disorder such as cancer (e.g., prostate cancer and breast cancer);

buserelin (Suprefact™, Suprecur™, Sanofi-Aventis), a ten amino acid peptide, and variants and derivatives thereof, which can be used in the conjugates, therapeutic delivery systems, and compositions described herein to treat proliferative disorders such as cancer (e.g., prostate cancer);

carfilzomib (PR-171, Proteolix Inc.), a four amino acid peptide, and variants and derivatives thereof, which can be used in the conjugates, therapeutic delivery systems, and compositions described herein to treat a proliferative disorder such as cancer (e.g., multiple myeloma, lymphoma, hematological neoplasms, and solid tumors);

CBP-501 (Takeda Pharmaceuticals), a twelve amino acid peptide, and variants and derivatives thereof, which can be used in the conjugates, therapeutic delivery systems, and compositions described herein to treat proliferative disorders such as cancer (e.g., lung cancer (e.g., small cell or non-small cell lung cancer) and mesothelioma);

cemadotin (LU-103793, Abbott Laboratories), a five amino acid peptide, and variants and derivatives thereof, which can be used in the conjugates, therapeutic delivery systems, and compositions described herein to treat proliferative disorders such as cancer;

cetrorelix (NS-75, Cetrotide™, AEterna Zentaris), a ten amino acid peptide, and variants and derivatives thereof, which can be used in the conjugates, therapeutic delivery systems, and compositions described herein to treat proliferative disorders such as benign protastatic hyperplasia, fibroids (e.g., uterine fibroids), cancer (e.g., breast cancer, ovarian cancer, prostate cancer);

chlorotoxin (TM-601. TransMolecular Inc.), a thirty-six amino acid peptide, and variants and derivatives thereof, which can be used in the conjugates, therapeutic delivery systems, and compositions described herein to treat proliferative disorders such as cancer (e.g., glioma);

cilengitide (EMD-121974, EMD-85189), a five amino acid peptide, and variants and derivatives thereof, which can be used in the conjugates, therapeutic delivery systems, and compositions described herein to treat proliferative disorders such as cancer (e.g., lung cancer (e.g., small cell or non-small cell lung cancer), glioblastoma, pancreatic cancer and prostate cancer);

CTCE-9908 (Chemokine Therapeutics Corp.), a seventeen amino acid peptide, and variants and derivatives thereof, which can be used in the conjugates, therapeutic delivery systems, and compositions described herein to treat a proliferative disorder such as cancer;

CVX-045 (Pfizer-Covx), and variants and derivatives thereof which can be used in the conjugates, therapeutic delivery systems, and compositions described herein to treat a proliferative disorder such as cancer (e.g., a solid tumor);

CVX-060 (Pfizer-Covx), and variants and derivatives thereof, which can be used in the conjugates, therapeutic delivery systems, and compositions described herein to treat a proliferative disorder such as cancer;

degarelix (FE 200486, Ferring Pharmaceuticals), a ten amino acid peptide, and variants and derivatives thereof, which can be used in the conjugates, therapeutic delivery systems, and compositions described herein to treat a proliferative disorder such as cancer (e.g., prostate cancer);

desolorelin (Somagard™, Shire), and variants and derivatives thereof, which can be used in the conjugates, therapeutic delivery systems, and compositions described herein to treat a proliferative disorder such as cancer (e.g., lymphoma (e.g., Non-Hodgkin lymphoma), brain cancer, prostate cancer, melanoma);

didemnin B (NSC-325319, PharmaMar), a six amino acid peptide, and variants and derivatives thereof, which can be used in the conjugates, therapeutic delivery systems, and compositions described herein to treat a proliferative disorder such as cancer (e.g., lymphoma (e.g., Non-Hodgkin lymphoma), brain cancer, melanoma);

DRF-7295 (Dabur India Ltd.), and variants and derivatives thereof, which can be used in the conjugates, therapeutic delivery systems, and compositions described herein to treat a proliferative disorder such as cancer (e.g., breast cancer and colorectal cancer);

edotreotide (SMT-487, OctreoTher™, Onaita™, Molecular Insight Pharmaceuticals), a cyclic seven amino acid peptide, and variants and derivatives thereof, which can be used in the conjugates, therapeutic delivery systems, and compositions described herein to treat a proliferative disorder such as cancer;

elisidepsin (PM-02734, Irvalec™, PharmaMar), and variants and derivatives thereof, which can be used in the conjugates, therapeutic delivery systems, and compositions described herein to treat a proliferative disorder such as cancer (e.g., lung cancer (e.g., small cell or non-small cell lung cancer));

epoetin alfa (Procrit™, Centocor Ortho Biotech) and variants and derivatives thereof, which can be used in the conjugates, therapeutic delivery systems, and compositions described herein to improve platelet counts in subjects undergoing myelosuppressive chemotherapy or to relieve anemia associated with chemotherapy;

EP-100 (Esperance Pharmaceuticals Inc.), a thirty-three amino acid peptide, and variants and derivatives thereof, which can be used in the conjugates, therapeutic delivery systems, and compositions described herein to treat a proliferative disorder such as cancer (e.g., prostate cancer);

ganirelix (Org-37462, RS-26306, Orgalutran™, Antagon™, Schering-Plough Corp), and variants and derivatives thereof, which can be used in the conjugates, therapeutic delivery systems, and compositions described herein to treat a proliferative disorder such as endometriosis and cancer (e.g., prostate cancer and breast cancer);

glutoxim (NOV-002, Pharma Vam), a six amino acid peptide, and variants and derivatives thereof, which can be used in the conjugates, therapeutic delivery systems, and compositions described herein to treat a proliferative disorder such as cancer (e.g., lung cancer (e.g., small cell or non-small cell lung cancer) and ovarian cancer);

goralatide (BIM-32001, Ipsen), a four amino acid peptide, and variants and derivatives thereof, which can be used in the conjugates, therapeutic delivery systems, and compositions described herein to treat a proliferative disorder such as cancer;

goserelin (ICI-118630, AstraZeneca), a ten amino acid peptide, and variants and derivatives thereof, which can be used in the conjugates, therapeutic delivery systems, and compositions described herein to treat a proliferative disorder such as cancer (e.g., prostate cancer, breast cancer, and uterine cancer);

histrelin (Vantas™, Johnson & Johnson), a nine amino acid peptide, and variants and derivatives thereof, which can be used in the conjugates, therapeutic delivery systems, and compositions described herein to treat a proliferative disorder such as cancer (e.g., prostate cancer);

labradimil (RMP-7, Cereport™, Johnson & Johnson), a nine amino acid peptide, and variants and derivatives thereof, which can be used in the conjugates, therapeutic delivery systems, and compositions described herein to treat a proliferative disorder such as cancer (e.g., glioma and brain cancer);

lanreotide (Somatuline™, Ipsen) an eight amino acid peptide, and variants and derivatives thereof, which can be used in the conjugates, therapeutic delivery systems, and compositions described herein to treat a proliferative disorder such as cancer (e.g. neuroblastoma);

leuprolide (Lupron™, Prostap™, Leuplin™, Enantone™, Takeda Pharmaceuticals), a nine amino acid peptide, and variants and derivatives thereof, which can be used in the conjugates, therapeutic delivery systems, and compositions described herein to treat a proliferative disorder such as fibroids (e.g., uterine fibroids) and cancer (e.g., prostate cancer);

LY-2510924 (AVE-0010, Sanofi-Aventis), a cyclic amino acid peptide, and variants and derivatives thereof, which can be used in the conjugates, therapeutic delivery systems, and compositions described herein to treat a proliferative disorder such as and cancer (e.g., breast cancer);

mifamurtide (Junovan™, Metpact™, Takeda Pharmaceuticals), a three amino acid peptide, and variants and derivatives thereof, which can be used in the conjugates, therapeutic delivery systems, and compositions described herein to treat a proliferative disorder such as cancer (e.g., osteosarcoma);

met-enkephalin (INNO-105, Innovive Pharmaceuticals Inc.), a five amino acid peptide, and variants and derivatives thereof, which can be used in the conjugates, therapeutic delivery systems, and compositions described herein to treat a proliferative disorder such as cancer (e.g., a solid tumor, pancreatic cancer);

muramyk tripeptide (Novartis), a three amino acid peptide, and variants and derivatives thereof, which can be used in the conjugates, therapeutic delivery systems, and compositions described herein to treat a proliferative disorder such as cancer;

nafarelin (RS-94991, Samynarel™, Nasanyl™, Synarel™, Synareia™, Roche), and variants and derivatives thereof, which can be used in the conjugates, therapeutic delivery systems, and compositions described herein to treat a proliferative disorder such as endometriosis and cancer (e.g., prostate cancer and breast cancer);

octreotide (SMS-201-995, Sandostatin™, Novartis), and variants and derivatives thereof, which can be used in the conjugates, therapeutic delivery systems, and compositions described herein to treat a proliferative disorder such as benign prostatic hyperplasia and cancer (e.g., prostate cancer);

ozarelix (D-63153, SPI-153, Spectrum Pharmaceuticals) a ten amino acid peptide, and variants and derivatives thereof, which can be used in the conjugates, therapeutic delivery systems, and compositions described herein to treat a proliferative disorder such as benign prostatic hyperplasia and cancer (e.g., prostate cancer);

pasireotide (SOM-230, Novartis) a six amino acid peptide, and variants and derivatives thereof, which can be used in the conjugates, therapeutic delivery systems, and compositions described herein to treat a proliferative disorder such as cancer (e.g., neuroendocrine tumors);

POL-6326 (Polyphor), and variants and derivatives thereof, which can be used in the conjugates, therapeutic delivery systems, and compositions described herein to treat a proliferative disorder such as cancer;

ramorelix (Hoe-013, Sanofi Aventis), a nine amino acid peptide, and variants and derivatives thereof, which can be used in the conjugates, therapeutic delivery systems, and compositions described herein to treat a proliferative disorder such as fibroids (e.g., uterine fibroids) and cancer (e.g., prostate cancer);

RC-3095 (AEterna Zentaris), a six amino acid peptide, and variants and derivatives thereof, which can be used in the conjugates, therapeutic delivery systems, and compositions described herein to treat a proliferative disorder such as cancer (e.g., a solid tumor);

Re-188-P-2045 (P2045, Neotide™, Bryan Oncor), an eleven amino acid peptide, and variants and derivatives thereof, which can be used in the conjugates, therapeutic delivery systems, and compositions described herein to treat a proliferative disorder such as cancer (e.g., lung cancer (e.g., small cell or non-small cell lung cancer));

romurtide (DJ-7041, Nopia™, Muroctasin™, Daiichi Sankyo), a two amino acid peptide, and variants and derivatives thereof, which can be used in the conjugates, therapeutic delivery systems, and compositions described herein to treat a proliferative disorder such as cancer;

YHI-501 (TZT-1027, Yakult Honsha KK), a two amino acid peptide, and variants and derivatives thereof, which can be used in the conjugates, therapeutic delivery systems, and compositions described herein to treat a proliferative disorder such as cancer (e.g., solid tumors);

Soblidotin (YHI-501, TZT-1027, Yakult Honsha KK), a two amino acid peptide, and variants and derivatives thereof, which can be used in the conjugates, therapeutic delivery systems, and compositions described herein to treat a proliferative disorder such as cancer;

SPI-1620 (Spectrum Pharmaceuticals), a fourteen amino acid peptide, and variants and derivatives thereof, which can be used in the conjugates, therapeutic delivery systems, and compositions described herein to treat a proliferative disorder such as cancer (e.g., solid tumors);

tabilautide (RP-56142, Sanofi Aventis), a three amino acid peptide, and variants and derivatives thereof, which can be used in the conjugates, therapeutic delivery systems, and compositions described herein to treat a proliferative disorder such as cancer;

TAK-448 (Takeda Pharmaceuticals), and variants and derivatives thereof, which can be used in the conjugates, therapeutic delivery systems, and compositions described herein to treat a proliferative disorder such as cancer (e.g., prostate cancer);

TAK-683 (Takeda Pharmaceuticals), and variants and derivatives thereof, which can be used in the conjugates, therapeutic delivery systems, and compositions described herein to treat a proliferative disorder such as cancer (e.g., prostate cancer);

tasidotin (ILX-651, BSF-223651, Genzyme), a five amino acid peptide, and variants and derivatives thereof, which can be used in the conjugates, therapeutic delivery systems, and compositions described herein to treat a proliferative disorder such as cancer (e.g., melanoma, prostate cancer and lung cancer (e.g., small cell or non-small cell lung cancer));

teverelix (EP-24332, Antarelix™, Ardana Biosciences), a ten amino acid peptide, and variants and derivatives thereof which can be used in the conjugates, therapeutic delivery systems, and compositions described herein to treat a proliferative disorder such as endometriosis, benign prostatic hyperplasia and cancer (e.g., prostate cancer);

tigapotide (PCK-3145, Kotinos Pharmaceuticals), a fifteen amino acid peptide, and variants and derivatives thereof, which can be used in the conjugates, therapeutic delivery systems, and compositions described herein to treat a proliferative disorder such as endometriosis, benign prostatic hyperplasia and cancer (e.g., prostate cancer);

thrombopoetin (Johnson & Johnson), and variants and derivatives thereof, which can be used in the conjugates, therapeutic delivery systems, and compositions described herein to improve platelet counts in subjects undergoing myelosuppressive chemotherapy or to relieve anemia associated with chemotherapy;

thymalfasin (Zadaxin™, Timosa™, Thymalfasin™, SciClone Pharmaceuticals), a twenty-eight amino acid peptide, and variants and derivatives thereof, which can be used in the conjugates, therapeutic delivery systems, and compositions described herein to treat a proliferative disorder such as cancer (e.g., melanoma, lung cancer (e.g., small cell or non-small cell lung cancer) and carcinoma (e.g., hepatocellular carcinoma));

TLN-232 (CAP-232, TT-232, Thallion Pharmaceuticals), a seven amino acid peptide, and variants and derivatives thereof, which can be used in the conjugates, therapeutic delivery systems, and compositions described herein to treat a proliferative disorder such as endometriosis, benign prostatic hyperplasia and cancer;

triptorelin (WY-42462, Debiopharma), a ten amino acid peptide, and variants and derivatives thereof, which can be used in the conjugates, therapeutic delivery systems, and compositions described herein to treat a proliferative disorder such as endometriosis, fibroids (e.g., uterine fibroids), benign prostatic hyperplasia and cancer (e.g., prostate cancer and breast cancer);

tyroserleutide (CMS-024, China Medical System), a three amino acid peptide, and variants and derivatives thereof, which can be used in the conjugates, therapeutic delivery systems, and compositions described herein to treat a proliferative disorder such as cancer (e.g., liver cancer (e.g., hepatocellular carcinoma); and

tyroservatide (CMS-024-02, China Medical Systems), a three amino acid peptide, and variants and derivatives thereof, which can be used in the conjugates, therapeutic delivery systems, and compositions described herein to treat a proliferative disorder such as cancer (e.g., lung cancer (e.g., small cell or non-small cell lung cancer)).

### Allergy, Inflammatory and Autoimmune Disorders

The disclosed CDP-therapeutic peptide conjugates may include peptides that treat or prevent allergy, inflammatory and/or autoimuune disorders. Exemplary therapeutic peptides that can be used in the disclosed CDP-therapeutic peptide conjugates include the following:

A-623 (AMG-623, Anthera Pharmaceuticals), a peptide, and variants and derivatives thereof, which can be used in the conjugates, therapeutic delivery systems, and compositions described herein to treat an allergy, inflammatory disorder, or immune disorder such as lupus erythematosus and chronic lymphocytic leukemia;

AG-284 (AnergiX.MS™, GlaxoSmithKline), a nineteen amino acid peptide, and variants and derivatives thereof, which can be used in the conjugates, therapeutic delivery systems, and compositions described herein to treat an allergy, inflammatory disorder, or immune disorder such as multiple sclerosis;

AI-502 (AutoImmune), a peptide, and variants and derivatives thereof, which can be used in the conjugates, therapeutic delivery systems, and compositions described herein to treat an allergy, inflammatory disorder, or immune disorder such as transplant rejection;

Allotrap 2702 (B-2702, Allotrap 2702™, Genzyme), a ten amino acid peptide, and variants and derivatives thereof, which can be used in the conjugates, therapeutic delivery systems, and compositions described herein to treat an allergy, inflammatory disorder, or immune disorder such as transplant rejection;

AZD-2315 (AstraZeneca), an eight amino acid peptide, and variants and derivatives thereof, which can be used in the conjugates, therapeutic delivery systems, and compositions described herein to treat an allergy, inflammatory disorder, or immune disorder such as rheumatoid arthritis;

Cnsnqic-Cyclic (802-2, Adeona Pharmaceuticals), a cyclic five amino acid peptide, and variants and derivatives thereof, which can be used in the conjugates, therapeutic delivery systems, and compositions described herein to treat an allergy, inflammatory disorder, or immune disorder such as Factor VIII deficiency, multiple sclerosis, and graft versus host disease;

delmitide (RDP-58, Genzyme), a ten amino acid peptide, and variants and derivatives thereof, which can be used in the conjugates, therapeutic delivery systems, and compositions described herein to treat an allergy, inflammatory disorder, or immune disorder such as inflammatory bowel disease, ulcerative colitis, and Crohn's disease;

dirucotide (MBP-8298, Eli Lilly and Co.), a seventeen amino acid peptide, and variants and derivatives thereof, which can be used in the conjugates, therapeutic delivery systems, and compositions described herein to treat an allergy, inflammatory disorder, or immune disorder such as multiple sclerosis;

disitertide (NAFB-001, P-144, ISDIN SA), a cyclic fourteen amino acid peptide, and variants and derivatives thereof, which can be used in the conjugates, therapeutic delivery systems, and compositions described herein to treat an allergy, inflammatory disorder, or immune disorder such as scleroderma;

dnaJP1 (AT-001, Adeona Pharmaceuticals), a fifteen amino acid peptide, and variants and derivatives thereof, which can be used in the conjugates, therapeutic delivery systems, and compositions described herein to treat an allergy, inflammatory disorder, or immune disorder such as rheumatoid arthritis;

edratide (TV-4710, Teva Pharmaceuticals), a twenty amino acid peptide, and variants and derivatives thereof which can be used in the conjugates, therapeutic delivery systems, and compositions described herein to treat an allergy, inflammatory disorder, or immune disorder such as systemic lupus erythematosus;

F-991 (Clinquest Inc.), a nine amino acid peptide, and variants and derivatives thereof, which can be used in the conjugates, therapeutic delivery systems, and compositions described herein to treat an allergy, inflammatory disorder, or immune disorder such as allergic asthma and skin disorder;

FAR-404 (Enkorten™, Farmacija d.o.o.), a peptide, and variants and derivatives thereof, which can be used in the conjugates, therapeutic delivery systems, and compositions described herein to treat an allergy, inflammatory disorder, or immune disorder such as functional bowel disorder, multiple sclerosis, rheumatoid arthritis, asthma, and systemic lupus erythematosus;

glaspimod (SKF-107647, GlaxoSmithKline), an eight amino acid peptide, and variants and derivatives thereof, which can be used in the conjugates, therapeutic delivery systems, and compositions described herein to treat an allergy, inflammatory disorder, or immune disorder such as leucopenia drug induced fungal infection, immune disorder, viral infection, bacterial infection, and immune deficiency;

glatiramer (COP-1, Copaxone™, Teva Phamiaceuticals), a peptide, and variants and derivatives thereof, which can be used in the conjugates, therapeutic delivery systems, and compositions described herein to treat an allergy, inflammatory disorder, or immune disorder such as glaucoma, Huntington's chorea, motor neuron disease, multiple sclerosis, and neurodegenerative disease;

glucosamyl muramyl tripeptide (Theramide™, DOR BioPharma Inc.), a three amino acid peptide, and variants and derivatives thereof, which can be used in the conjugates, therapeutic delivery systems, and compositions described herein to treat an allergy, inflammatory disorder, or immune disorder such as herpesvirus infection, postoperative infections, psoriasis, respiratory tract disorders (e.g. lung disorders), and tuberculosis;

GMDP (Likopid™, Licopid™, Arana Therapeutics), a two amino acid peptide, and variants and derivatives thereof, which can be used in the conjugates, therapeutic delivery systems, and compositions described herein to treat an allergy, inflammatory disorder, or immune disorder such as herpesvirus infection, postoperative infections, psoriasis, respiratory tract disorders (e.g., lung disorders), and tuberculosis;

icatibant (JE-049, HOE-140, Firazyr™, Shire), an eight amino acid peptide, and variants and derivatives thereof, which can be used in the conjugates, therapeutic delivery systems, and compositions described herein to treat an allergy, inflammatory disorder, or immune disorder such as hereditary angioedema, rhinitis, asthma, osteoarthritis, pain, and liver cirrhosis;

IPP-201101 (Lupuzor™, ImmuPharma Ltd.), a peptide, and variants and derivatives thereof, which can be used in the conjugates, therapeutic delivery systems, and compositions described herein to treat an allergy, inflammatory disorder, or immune disorder such as systemic lupus erythematosus;

lusupultide (Venticute, Nycomed GmbH), a peptide, and variants and derivatives thereof, which can be used in the conjugates, therapeutic delivery systems, and compositions described herein, and act as a pulmonary surfactant for the treatment of pulmonary distress, such as asthma.

MS peptide (Briana Bio-Tech Inc.), a peptide, and variants and derivatives thereof, which can be used in the conjugates, therapeutic delivery systems, and compositions described herein to treat an allergy, inflammatory disorder, or immune disorder such as multiple sclerosis;

NPC-567 (Johnson & Johnson) and variants and derivatives thereof, which can be used in the conjugates, therapeutic delivery systems, and compositions described herein to treat an allergy, inflammatory disorder, or immune disorder such as asthma;

Org-42982 (AG-4263, AnergiX.RA™, GlaxoSmithKline), a thirteen amino acid peptide, and variants and derivatives thereof, which can be used in the conjugates, therapeutic delivery systems, and compositions described herein to treat an allergy, inflammatory disorder, or immune disorder such as rheumatoid arthritis;

pentigetide (TA-521, Pentyde™, Bausch & Lomb), a five amino acid peptide, and variants and derivatives thereof, which can be used in the conjugates, therapeutic delivery systems, and compositions described herein to treat an allergy, inflammatory disorder, or immune disorder such as allergic rhinitis and allergic conjunctivitis;

PI-0824 (Genzyme), a nineteen amino acid peptide, and variants and derivatives thereof, which can be used in the conjugates, therapeutic delivery systems, and compositions described herein to treat an allergy, inflammatory disorder, or immune disorder such as pemphigus vulgaris;

PI-2301 (Peptimmune), a peptide, and variants and derivatives thereof, which can be used in the conjugates, therapeutic delivery systems, and compositions described herein to treat an allergy, inflammatory disorder, or immune disorder such as multiple sclerosis;

PLD-116 (Barr Pharmaceuticals Inc.), a fifteen amino acid peptide, and variants and derivatives thereof, which can be used in the conjugates, therapeutic delivery systems, and compositions described herein to treat an allergy, inflammatory disorder, or immune disorder such as ulcerative colitis;

PMX-53 (Arana Therapeutics), a cyclic six amino acid peptide, and variants and derivatives thereof, which can be used in the conjugates, therapeutic delivery systems, and compositions described herein to treat an allergy, inflammatory disorder, or immune disorder such as inflammation, rheumatoid arthritis, and psoriasis;

PTL-0901 (Acambis plc), a nine amino acid peptide, and variants and derivatives thereof, which can be used in the conjugates, therapeutic delivery systems, and compositions described herein to treat an allergy, inflammatory disorder, or immune disorder such as allergic rhinitis;

RA peptide (Acambis plc), a four amino acid peptide, and variants and derivatives thereof, which can be used in the conjugates, therapeutic delivery systems, and compositions described herein to treat an allergy, inflammatory disorder, or immune disorder such as rheumatoid arthritis;

TCMP-80 (Elan Corp.), a two amino acid peptide, and variants and derivatives thereof, which can be used in the conjugates, therapeutic delivery systems, and compositions described herein to treat an allergy, inflammatory disorder, or immune disorder;

thymodepressin (Immunotech Developments), a two amino acid peptide, and variants and derivatives thereof, which can be used in the conjugates, therapeutic delivery systems, and compositions described herein to treat an allergy, inflammatory disorder, or immune disorder such as recurring autoimmune cytopenia (1, 2, 3 lineage), hypoplastic anemia, rheumatoid arthritis, and psoriasis;

thymopentin (TP-5, Timunox™, Johnson & Johnson), a five amino acid peptide, and variants and derivatives thereof, which can be used in the conjugates, therapeutic delivery systems, and compositions described herein to treat an allergy, inflammatory disorder, or immune disorder such as lung infection, rheumatoid arthritis, HIV infection, and primary immunodeficiencies;

tiplimotide (NBI-5788, Neurocrine Biosciences Inc.), a seventeen amino acid peptide, and variants and derivatives thereof, which can be used in the conjugates, therapeutic delivery systems, and compositions described herein to treat an allergy, inflammatory disorder, or immune disorder such as multiple sclerosis;

ularitide (CDD-95-126, ESP-305, CardioBiss™, Nephrobiss™, EKR Therapeutics), a cyclic thirty-two amino acid peptide, and variants and derivatives thereof, which can be used in the conjugates, therapeutic delivery systems, and compositions described herein to treat an allergy, inflammatory disorder, or immune disorder such as asthma; and

ZP-1848 (Zealand Pharma), a peptide, and variants and derivatives thereof, which can be used in the conjugates, therapeutic delivery systems, and compositions described herein to treat an allergy, inflammatory disorder, or immune disorder.

### Cardiovascular Disease

The disclosed CDP-therapeutic peptide conjugates may be useful in the prevention and treatment of cardiovascular disease.

Exemplary therapeutic peptides that can be used in the disclosed CDP-therapeutic peptide conjugates include the following:

AC-2592 (Betatropin™, Amylin Pharmaceuticals), a thirty amino acid peptide, and variants and derivatives thereof, which can be used in the conjugates, therapeutic delivery systems, and compositions described herein to treat a cardiovascular disorder such as heart failure;

AC-625 (Amylin Pharmaceuticals), a peptide, and variants and derivatives thereof, which can be used in the conjugates, therapeutic delivery systems, and compositions described herein to treat a cardiovascular disorder such as hypertension;

anaritide (Auriculin™, Johnson & Johnson), a cyclic twenty-five amino acid peptide, and variants and derivatives thereof, which can be used in the conjugates, therapeutic delivery systems, and compositions described herein to treat a cardiovascular disorder such as renal failure, heart failure, and hypertension;

APL-180 (Novartis), a peptide, and variants and derivatives thereof, which can be used in the conjugates, therapeutic delivery systems, and compositions described herein to treat a cardiovascular disorder such as coronary disorder;

atriopeptin (Astellas Pharma), a twenty-five amino acid peptide, and variants and derivatives thereof, which can be used in the conjugates, therapeutic delivery systems, and compositions described herein to treat a cardiovascular disorder;

BGC-728 (BTG plc), a cyclic peptide, and variants and derivatives thereof, which can be used in the conjugates, therapeutic delivery systems, and compositions described herein to treat a cardiovascular disorder such as myocardial infarction and cerebrovascular ischemia;

carperitide (SUN-4936, HANP™, Daiichi Sankyo), a cyclic peptide, and variants and derivatives thereof, which can be used in the conjugates, therapeutic delivery systems, and compositions described herein to treat a cardiovascular disorder such as heart failure;

CD-NP (Nile Therapeutics), a forty-one amino acid peptide, and variants and derivatives thereof, which can be used in the conjugates, therapeutic delivery systems, and compositions described herein to treat a cardiovascular disorder such as heart failure;

CG-77X56 (Cardeva™, Teva Pharmaceuticals), a peptide, and variants and derivatives thereof, which can be used in the conjugates, therapeutic delivery systems, and compositions described herein to treat a cardiovascular disorder such as heart failure;

D-4F (APP-018, Novartis), an eighteen amino acid peptide, and variants and derivatives thereof, which can be used in the conjugates, therapeutic delivery systems, and compositions described herein to treat a cardiovascular disorder such as atherosclerosis;

danegaptide (ZP-1609, WAY-261134, GAP-134, Zealand Pharma), a two amino acid peptide, and variants and derivatives thereof, which can be used in the conjugates, therapeutic delivery systems, and compositions described herein to treat a cardiovascular disorder such as heart arrhythmia;

DMP-728 (DU-728, Bristol-Myers Squibb), a cyclic three amino acid peptide, and variants and derivatives thereof, which can be used in the conjugates, therapeutic delivery systems, and compositions described herein to treat a cardiovascular disorder such as thrombosis coronary thrombosis);

efegatran (LY-294468, Eli Lilly and Co.), a three amino acid peptide, and variants and derivatives thereof, which can be used in the conjugates, therapeutic delivery systems, and compositions described herein to treat a cardiovascular disorder such as myocardial infarction and thrombosis (e.g., coronary thrombosis);

EMD-73495 (Merck kGaA), a peptide, and variants and derivatives thereof, which can be used in the conjugates, therapeutic delivery systems, and compositions described herein to treat a cardiovascular disorder;

eptifibatide (C68-22, Integrelin™, Integrilin™, Takeda Pharmaceuticals), a cyclic six amino acid peptide, and variants and derivatives thereof, which can be used in the conjugates, therapeutic delivery systems, and compositions described herein to treat a cardiovascular disorder such as acute coronary syndrome, myocardial infarction, and unstable angina pectoris;

ET-642 (RLT-peptide, Pfizer), a twenty-two amino acid peptide, and variants and derivatives thereof, which can be used in the conjugates, therapeutic delivery systems, and compositions described herein to treat a cardiovascular disorder such as atherosclerosis;

FE 202158 (Ferring Pharmaceuticals), a cyclic nine amino acid peptide, and variants and derivatives thereof, which can be used in the conjugates, therapeutic delivery systems, and compositions described herein to treat a cardiovascular disorder such as vasodilatory hypotension (e.g., sepsis and intradialytic hypotension);

FX-06 (Ikaria), a peptide, and variants and derivatives thereof, which can be used in the conjugates, therapeutic delivery systems, and compositions described herein to treat a cardiovascular disorder such as reperfusion injury;

icrocaptide (ITF-1697, Italfarmaco), a four amino acid peptide, and variants and derivatives thereof, which can be used in the conjugates, therapeutic delivery systems, and compositions described herein to treat a cardiovascular disorder such as respiratory distress syndrome;

KAI-1455 (KAI Pharmaceuticals), a twenty amino acid peptide, and variants and derivatives thereof, which can be used in the conjugates, therapeutic delivery systems, and compositions described herein to treat a cardiovascular disorder such as cardiovascular surgery cytoprotection;

KAI-9803 (KAI Pharmaceuticals), a twenty-three amino acid peptide, and variants and derivatives thereof, which can be used in the conjugates, therapeutic delivery systems, and compositions described herein to treat a cardiovascular disorder such as myocardial infarction, reperfusion injury, and coronary artery disease;

L-346670 (Merck & Co. Inc.), a cyclic twenty-six amino acid peptide, and variants and derivatives thereof, which can be used in the conjugates, therapeutic delivery systems, and compositions described herein to treat a cardiovascular disorder such as hypertension;

L-364343 (Merck & Co. Inc.), a cyclic twenty-nine amino acid peptide, and variants and derivatives thereof, which can be used in the conjugates, therapeutic delivery systems, and compositions described herein to treat a cardiovascular disorder such as hypertension;

LSI-518P (Lipid Sciences Inc.), a peptide, and variants and derivatives thereof, which can be used in the conjugates, therapeutic delivery systems, and compositions described herein to treat a cardiovascular disorder;

nesiritide (Noratak™, Natrecor™, Johnson & Johnson), a thirty-two amino acid peptide, and variants and derivatives thereof, which can be used in the conjugates, therapeutic delivery systems, and compositions described herein to treat a cardiovascular disorder such as heart failure;

peptide rennin inhibitor (Pfizer), a peptide, and variants and derivatives thereof, which can be used in the conjugates, therapeutic delivery systems, and compositions described herein to treat a cardiovascular disorder;

PL-3994 (Palatin Technologies), a fifteen amino acid peptide, and variants and derivatives thereof, which can be used in the conjugates, therapeutic delivery systems, and compositions described herein to treat a cardiovascular disorder such as hypertension and heart failure;

rotigaptide (ZP-123, GAP-486, Zealand Pharma), a six amino acid peptide, and variants and derivatives thereof, which can be used in the conjugates, therapeutic delivery systems, and compositions described herein to treat a cardiovascular disorder such as ventricular arrhythmia and atrial fibrillation;

saralasin (P-113, Sarenin™, Procter & Gamble), an eight amino acid peptide, and variants and derivatives thereof, which can be used in the conjugates, therapeutic delivery systems, and compositions described herein to treat a cardiovascular disorder;

SKF-105494 (GlaxoSmithKline), a cyclic seven amino acid peptide, and variants and derivatives thereof, which can be used in the conjugates, therapeutic delivery systems, and compositions described herein to treat a cardiovascular disorder such as hypertension;

terlakiren (CP-80794, Pfizer), a two amino acid peptide, and variants and derivatives thereof, which can be used in the conjugates, therapeutic delivery systems, and compositions described herein to treat a cardiovascular disorder such as hypertension;

thymalfasin (Zadaxin™, Timosa™, Thymalfasin™, SciClone Pharmaceuticals), a twenty-eight amino acid peptide, and variants and derivatives thereof, which can be used in the conjugates, therapeutic delivery systems, and compositions described herein to treat a cardiovascular disorder such as angiogenesis disorder;

tridecactide (AP-214, Action Pharma), a ten amino acid peptide, and variants and derivatives thereof, which can be used in the conjugates, therapeutic delivery systems, and compositions described herein to treat a cardiovascular disorder such as reperfusion injury and renal disease;

ularitide (CDD-95-126, ESP-305, CardioBiss™, Nephrobiss™, EKR Therapeutics), a cyclic thirty-two amino acid peptide, and variants and derivatives thereof, which can be used in the conjugates, therapeutic delivery systems, and compositions described herein to treat a cardiovascular disorder such as heart failure and renal failure;

urocortin II (Neurocrine Biosciences Inc.), a thirty-eight amino acid peptide, and variants and derivatives thereof, which can be used in the conjugates, therapeutic delivery systems, and compositions described herein to treat a cardiovascular disorder such as heart failure; and

ZP-120 (Zealand Pharma), a twelve amino acid peptide, and variants and derivatives thereof, which can be used in the conjugates, therapeutic delivery systems, and compositions described herein to treat a cardiovascular disorder such as isolated systolic hypertension and heart failure.

### Renal Disease

The disclosed CDP-therapeutic peptide conjugates are useful in treating kidney disorders, e.g., a kidney disorder described herein.

The therapeutic peptide can be, e.g., a peptide agonist of GHRH receptor, a peptide agonist of ANP receptor, a peptide agonist of AVP receptora peptide agonist of CALC receptor, a peptide agonist of CRH receptor, a peptide agonist of SST receptor, a peptide agonist of IL-2 receptor, and a peptide agonist of MC receptor.

Examples of therapeutic peptides that can be used in the claimed conjugates, particles and compositions include the following:

AKL-0707 (Aleka Pharma) a twenty-nine amino acid peptide, and variants and derivatives thereof, which can be used in the conjugates, therapeutic delivery systems, and compositions described herein to treat a kidney disorder, e.g., kidney dysfunction associated with a lipid metabolism disorder;

aniritide (Johnson & Johnson) a twenty-five amino acid cyclic peptide, and variants and derivatives thereof, which can be used in the conjugates, therapeutic delivery systems, and compositions described herein to treat a kidney disorder, e.g., renal failure;

BIM-44002 (Ipsen) a twenty-eight amino acid peptide, and variants and derivatives thereof, which can be used in the conjugates, therapeutic delivery systems, and compositions described herein to treat a kidney disorder, e.g., renal failure, e.g., hypercalcemia associated with renal failure;

human calcitonin (Cibacalcin®, Novartis) a thirty-two amino acid peptide, and variants and derivatives thereof, which can be used in the conjugates, therapeutic delivery systems, and compositions described herein to treat a kidney disorder, e.g., renal failure, e.g., hypercalcemia associated with renal failure;

salmon calcitonin (Calcimar®, Sanofi-Aventis) a thirty-two amino acid cyclic peptide, and variants and derivatives thereof, which can be used in the conjugates, therapeutic delivery systems, and compositions described herein to treat a kidney disorder, e.g., renal failure, e.g., hypercalcemia associated with renal failure;

C-peptide (SPM-933, Cebix) a thirty-one amino acid linear peptide, and variants and derivatives thereof, which can be used in the conjugates, therapeutic delivery systems, and compositions described herein to treat a kidney disorder, e.g., nephropathy, e.g., diabetic nephropathy;

desmopressin (Minirin®, DDAVP®, Octostim®, Ferring Pharmaceuticals) a nine amino acid cyclic peptide, and variants and derivatives thereof, which can be used in the conjugates, therapeutic delivery systems, and compositions described herein to treat a kidney disorder, e.g., nephropathy, e.g., diabetic nephropathy;

DG-3173 (PTR-3173, Somatoprim®, DeveloGen) an eight amino acid cyclic peptide, and variants and derivatives thereof, which can be used in the conjugates, therapeutic delivery systems, and compositions described herein to treat a kidney disorder, e.g., nephropathy, e.g., diabetic nephropathy;

EA-230 (Exponential Biotherapies) a four amino acid linear peptide, and variants and derivatives thereof, which can be used in the conjugates, therapeutic delivery systems, and compositions described herein to treat a kidney disorder, e.g., renal failure;

elcatonin (Sidinuo®, Elcitonin®, Asahi Kasei Pharma) a thirty-one amino acid cyclic peptide, and variants and derivatives thereof, which can be used in the conjugates, therapeutic delivery systems, and compositions described herein to treat a kidney disorder, e.g., renal failure, e.g., hypercalcemia associated with renal failure;

KAI-4169 (KAI Pharmaceuticals, Inc.; see U.S. Patent Application Publication No. 2011/0028394), a seven amino acid peptide, and variants and derivatives thereof including, for example, peptides that comprise sequences with cell-penetrating characteristics (see the amino acid sequences, e.g., SEQ ID NO:2, disclosed in U.S. Patent Application Publication No. 2011/0028394, which is herein incorporated by reference in its entirety), which can be used in the conjugates, therapeutic delivery systems, and compositions described herein to treat kidney disease, for example chronic kidney disease, e.g., hyperparathyroidism, for example, secondary hyperparathyroidism, associated with patients with chronic kidney disease;

lypressin (Diapid®, Novartis) a nine amino acid cyclic peptide, and variants and derivatives thereof, which can be used in the conjugates, therapeutic delivery systems, and compositions described herein to treat a kidney disorder, e.g., diabetes insipidus;

terlipressin (Glypressin®, Ferring Pharmaceuticals) a twelve amino acid cyclic peptide, and variants and derivatives thereof, which can be used in the conjugates, therapeutic delivery systems, and compositions described herein to treat a kidney disorder, e.g., hepatorenal syndrome;

tridecactide (AP-214, Action Pharma) a ten amino acid linear peptide, and variants and derivatives thereof, which can be used in the conjugates, therapeutic delivery systems, and compositions described herein to treat a kidney disorder; and

ularitide (CDD-95-126, ESP-305, CardioBiss®, Nephrobiss®, EKR Therapeutics) a thirty-two amino acid cyclic peptide, and variants and derivatives thereof, which can be used in the conjugates, therapeutic delivery systems, and compositions described herein to treat a kidney disorder, e.g., renal failure.

### Metabolic and Other Disorders

The disclosed CDP-therapeutic peptide conjugates may be useful in the prevention and treatment of metabolic disorders.

In some embodiments, the therapeutic peptide is a hormone. Examples of hormones include enkephalin, GLP-1 (e.g., GLP-1 (7-37), GLP-1 (7-36)), GLP-2, insulin, insulin-like growth factor-1, insulin-like growth factor-2, orexin A, orexin B, neuropeptide Y, growth hormone-releasing hormone, thryotropin-releasing hormone, cholecystokinin, melanocyte-stimulating hormone, corticotrophin-releasing factor, melanin concentrating hormone, galanin, bombesin, calcitonin gene related peptide, neurotensin, endorphin, dynorpin, the C-peptide of proinsulin, and irisin.

Preferably, the therapeutic peptide is an anti-diabetogenic peptide. An anti-diabetogenic peptide includes a peptide having one or more of the following activities: 1) ability to increase insulin secretion; 2) ability to increase insulin biosynthesis; 3) ability to decrease glucagon secretion; 4) ability to delay gastric emptying; 5) reduce hepatic gluconeogenesis; 6) improve insulin sensitivity; 7) improve glucose sensing by the beta cell; 8) enhance glucose disposal; 9) reduce insulin resistance; and 10) promote beta cell function or viability. Examples of anti-diabetogenic peptides include glucagon-like peptide-1 (GLP-1), insulin, insulin-like growth factor-1, insulin-like growth factor-2, exedin-4, gastric inhibitory polypeptide, irisin and variants and derivatives thereof. Variants of some of the small peptides listed above are known. For example, known variants of GLP-1 include, for example, GLP-1 (7-36), GLP-1 (7-37), Gln⁹-GLP-1 (7-37), Thr¹⁶-Lys¹⁸-GLP-1 (7-37), Lys¹⁸-GLP-1 (7-37) and Gly⁸-GLP-1. Derivatives include, for example, acid addition salts, carboxylate salts, lower alkyl esters, and amides such as those described in PCT Publication WO 91/11457.

Exemplary therapeutic peptides include:

A-71378 (Abbott Laboratories) which is a six amino acid peptide (and variants and derivatives thereof) that can be used in the conjugates, therapeutic delivery systems, and compositions described herein to treat metabolic disorders such as obesity;

PYY 3-36 (Amylin Pharmaceuticals) a thirty-four amino acid peptide (and variants and derivatives thereof) that can be used in the conjugates, therapeutic delivery systems, and compositions described herein to treat metabolic disorders such as obesity;

AC-253 (Antam, Amylin Pharmaceuticals), and variants and derivatives thereof, which can be used in the conjugates, therapeutic delivery systems, and compositions described herein to treat metabolic disorders such as diabetes (e.g., type 1 diabetes, type 2 diabetes, and/or gestational diabetes) and obesity;

albiglutide (GSK-716155, Syncria, GlaxoSmithKline), and variants and derivatives thereof, which can be used in the conjugates, therapeutic delivery systems, and compositions described herein to treat metabolic disorders such as diabetes (e.g., type 1 diabetes, type 2 diabetes, gestational diabetes);

AKL-0707 (LAB GHRH, Akela Pharma), a 29 amino acid peptide, and variants and derivatives thereof, which can be used in the conjugates, therapeutic delivery systems, and compositions described herein to treat metabolic disorders such as lipid metabolism disorder and malnutrition;

AOD-9604 (Metabolic Pharmaceuticals, Ltd.), a cyclic 16 amino acid peptide, and variants and derivatives thereof, which can be used in the conjugates, therapeutic delivery systems, and compositions described herein to treat metabolic disorders such as obesity;

BAY-73-7977 (Bayer AG), and variants and derivatives thereof, which can be used in the conjugates, therapeutic delivery systems, and compositions described herein to treat a metabolic disorder such as diabetes (e.g., type 1 diabetes, type 2 diabetes, and gestational diabetes);

BMS-686117 (Bristol-Myers Squibb), an eleven amino acid peptide, and variants and derivatives thereof, which can be used in the conjugates, therapeutic delivery systems, and compositions described herein to treat metabolic disorders such as diabetes (e.g. type 1 diabetes, type 2 diabetes, and gestational diabetes);

BIM-44002 (Ipsen), a twenty-eight amino acid peptide, and variants and derivatives thereof, which can be used in the conjugates, therapeutic delivery systems, and compositions described herein to treat metabolic disorders such as hypercalcemia;

CVX-096 (Pfizer-Covx), and variants and derivatives thereof, which can be used in the conjugates, therapeutic delivery systems, and compositions described herein to treat a metabolic disorder such as diabetes (e.g. type 1 diabetes, type 2 diabetes, and gestational diabetes);

davalintide (AC-2307, Amylin Pharmaceuticals), a cyclic thirty amino acid peptide, and variants and derivatives thereof, which can be used in the conjugates, therapeutic delivery systems, and compositions described herein to treat a metabolic disorder such as obesity;

AC-2993 (LY-2148568, Byetta™, Amylin Pharmaceuticals) a thirty-eight amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes) and obesity;

exsulin (INGAP peptide, Exsulin), a fifteen amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

glucagon (Glucogen™, Novo Nordisk), a twenty-nine amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

Irisin (Ember Theraputics, Inc., see Bostrom et al., 2012, Nature 481(7382):463-468), a 112 amino acid peptide, and variants and derivatives thereof, which can be used in the conjugates, therapeutic delivery systems, and compositions described herein to treat a metabolic disorder such as obesity, disorders associated with glucose homeostasis, e.g., diabetes (for example, type 1 diabetes, type 2 diabetes, gestational diabetes), and disorders that are improved with exercise.

ISF402 (Dia-B Tech), a four amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

KAI-4169 (KAI Pharmaceuticals, Inc.: see U.S. Patent Application Publication No. 2011/0028394), a seven amino acid peptide, and variants and derivatives thereof including, for example, peptides that comprise sequences with cell-penetrating characteristics (see the amino acid sequences, e.g., SEQ ID NO:2, disclosed in U.S. Patent Application Publication No. 2011/0028394, which is herein incorporated by reference in its entirety), which can be used in the conjugates, therapeutic delivery systems, and compositions described herein to treat hyperparathyroidism, disorders involving abnormal calcium levels (e.g., hypercalcemia), and bone disease;

larazotide (AT-1001, SPD-550, Alba Therapeutics Corp), an eight amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

linaclotide (Ironwood Pharmaceuticals, Inc.), a 14 amino acid cyclic peptide, and variants and derivatives thereof, which can be used in the conjugates, therapeutic delivery systems, and compositions described herein to treat gastrointestinal disorders, for example, irritable bowel syndrome, e.g., irritable bowel syndrome with constipation, and constipation, e.g., chronic constipation.

liraglutide (Victoza™, Novo Nordisk), a thirty-one amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes) and obesity;

lixisenatide (AVE-0010, ZP-10, Sanofi Aventis), a forty-four amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

LY-2189265 (Eli Lilly & Co.), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

LY-548805 (Eli Lilly & Co.), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

NBI-6024 (Neurocrine Biosciences, Inc.), a fifteen amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

obinepitide (7TM Pharma), a thirty-six amino acid peptide, and variants and derivatives thereof, which can be used in the conjugates, therapeutic delivery systems, and compositions described herein to treat a metabolic disorder such as obesity;

peptide YY (3-36) (MDRNA Inc.), a thirty-four amino acid peptide, and variants and derivatives thereof, which can be used in the conjugates, therapeutic delivery systems, and compositions described herein to treat a metabolic disorder such as obesity;

pramlintide (Symlin™, Amylin Pharmaceuticals), a cyclic thirty four amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes) and obesity;

R-7089 (Roche), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

semaglutide (NN-9535, Novo Nordisk), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

somatropin (Nutropin, Genentech), a 191 amino acid peptide, and variants and derivatives thereof, which can be used in the conjugates, therapeutic delivery systems, and compositions described herein to treat metabolic disorders such as growth disorders, e.g., Turner syndrome;

SST analog (Merck & Co. Inc.), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

SUN-E7001 (CS-872, Daiichi Sankyo), a thirty amino acid peptide, and variants and derivatives thereof, which can be used in the conjugates, therapeutic delivery systems, and compositions described herein to treat a metabolic disorder such as diabetes (e.g., type I diabetes, type 2 diabetes, gestational diabetes);

taspoglutide (BIM-51077, Roche), a thirty amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

tesamorelin (TH-9507, Theratechnologies), a forty-four amino acid peptide, and variants and derivatives thereof, which can be used in the conjugates, therapeutic delivery systems, and compositions described herein to treat a metabolic disorder such as somatotrophin deficiency, muscle wasting and lipodystrophy;

TH-0318 (OctoPlus NV), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

TKS-1225 (oxyntomodulin, Wyeth), a thirty-seven amino acid peptide, and variants and derivatives thereof, which can be used in the conjugates, therapeutic delivery systems, and compositions described herein to treat a metabolic disorder such as obesity;

TM-30339 (7TM Pharma), and variants and derivatives thereof, which can be used in the conjugates, therapeutic delivery systems, and compositions described herein to treat a metabolic disorder such as obesity;

TT-223 (E1-INT, Eli Lilly & Co.), and variants and derivatives thereof, which can be used in the conjugates, therapeutic delivery systems, and compositions described herein to treat a metabolic disorder such as diabetes (e.g., type 1 diabetes, type 2 diabetes, gestational diabetes);

unacylated ghrelin (AZP-01, Alize Pharma), a twenty-eight amino acid peptide, and variants and derivatives thereof, which can be used in the conjugates, therapeutic delivery systems, and compositions described herein to treat a metabolic disorder such as diabetes (e.g., type 1 diabetes, type 2 diabetes, gestational diabetes); and

urocortin II (Neurocrine Biosciences Inc.), a thirty-eight amino acid peptide, and variants and derivatives thereof, which can be used in the conjugates, therapeutic delivery systems, and compositions described herein to treat a metabolic disorder such as obesity.

### Infectious Disease

The CDP-therapeutic peptide conjugates described herein can include a peptide that treats or prevents infectious disease. Exemplary therapeutic peptides that can be used in the disclosed CDP-therapeutic peptide conjugates include the following:

albuvirtide (Frontier Biotechnologies), a peptide, and variants and derivatives thereof, which can be used in the conjugates, therapeutic delivery systems, and compositions described herein to treat a microbial disorder or viral disorder such as HIV infection;

ALG-889 (Allergene Inc.), a sixteen amino acid peptide, and variants and derivatives thereof, which can be used in the conjugates, therapeutic delivery systems, and compositions described herein to treat a microbial disorder or viral disorder such as HIV infection and immune disorder;

alloferon (Allokine-alpha™, EntoPharm Co. Ltd.), a thirteen amino acid peptide, and variants and derivatives thereof, which can be used in the conjugates, therapeutic delivery systems, and compositions described herein to treat a microbial disorder or viral disorder such as hepatitis B virus infection, hepatitis C virus infection, herpesvirus infection, and cancer;

ALX-40-AC (NPS Pharmaceuticals), a nine amino acid peptide, and variants and derivatives thereof, which can be used in the conjugates, therapeutic delivery systems, and compositions described herein to treat a microbial disorder or viral disorder such as HIV infection;

CB-182804 (Cubist Pharmaceuticals), a peptide, and variants and derivatives thereof, which can be used in the conjugates, therapeutic delivery systems, and compositions described herein to treat a microbial disorder or viral disorder such as multidrug-resistant Gram negative bacterial infection;

CB-183315 (Cubist Pharmaceuticals), a peptide, and variants and derivatives thereof, which can be used in the conjugates, therapeutic delivery systems, and compositions described herein to treat a microbial disorder or viral disorder such as Clostridium difficile-associated diarrhea;

CZEN-002 (Migami), a polymeric eight amino acid peptide, and variants and derivatives thereof, which can be used in the conjugates, therapeutic delivery systems, and compositions described herein to treat a microbial disorder or viral disorder such as vulvovaginal candidiasis;

enfuvirtide (T-20, Fuzeon™, Roche), a thirty-six amino acid peptide, and variants and derivatives thereof which can be used in the conjugates, therapeutic delivery systems, and compositions described herein to treat a microbial disorder or viral disorder such as HIV infection;

glucosamyl muramyl tripeptide (Theramide™, DOR BioPharma Inc.), a three amino acid peptide, and variants and derivatives thereof, which can be used in the conjugates, therapeutic delivery systems, and compositions described herein to treat a microbial disorder or viral disorder such as herpesvirus infection, postoperative infections, psoriasis, respiratory tract disorders (e.g., lung disorders), and tuberculosis;

GMDP (Likopid™, Licopid™, Arana Therapeutics), a two amino acid peptide, and variants and derivatives thereof, which can be used in the conjugates, therapeutic delivery systems, and compositions described herein to treat a microbial disorder or viral disorder such as herpesvirus infection, postoperative infections, psoriasis, respiratory tract disorders (e.g., lung disorders), and tuberculosis;

golotimod (SCV-07, SciClone Pharmaceuticals), a two amino acid peptide, and variants and derivatives thereof, which can be used in the conjugates, therapeutic delivery systems, and compositions described herein to treat a microbial disorder or viral disorder such as hepatitis C, viral infection, and tuberculosis;

GPG-NH2 (Tripep), a three amino acid peptide, and variants and derivatives thereof, which can be used in the conjugates, therapeutic delivery systems, and compositions described herein to treat a microbial disorder or viral disorder such as HIV infection;

hLF (1-11) (AM-Pharma Holding BV), an eleven amino acid peptide, and variants and derivatives thereof, which can be used in the conjugates, therapeutic delivery systems, and compositions described herein to treat a microbial disorder or viral disorder such as bacterial infection, mycoses, bacteremia, and candidemia;

IMX-942 (Inimex Pharmaceuticals), a peptide, and variants and derivatives thereof, which can be used in the conjugates, therapeutic delivery systems, and compositions described herein to treat a microbial disorder or viral disorder such as hospital-acquired bacterial infections:

iseganan (IB-367, Ardea Biosciences Inc.), a cyclic sixteen amino acid peptide, and variants and derivatives thereof, which can be used in the conjugates, therapeutic delivery systems, and compositions described herein to treat a microbial disorder or viral disorder such as stomatitis and nosocomial pneumonia;

murabutide (VA-101, CY-220 Sanoti-Aventis), a two amino acid peptide, and variants and derivatives thereof, which can be used in the conjugates, therapeutic delivery systems, and compositions described herein to treat a microbial disorder or viral disorder such as hepatitis virus infection and HIV infection;

neogen (Neogen™, Immunotech Developments), a peptide, and variants and derivatives thereof, which can be used in the conjugates, therapeutic delivery systems, and compositions described herein to treat a microbial disorder or viral disorder such as viral infection, bacterial infection, and hemopoietic disorder;

NP-213 (Novexatin™, NovaBiotics), a cyclic amino acid peptide, and variants and derivatives thereof, which can be used in the conjugates, therapeutic delivery systems, and compositions described herein to treat a microbial disorder or viral disorder such as onychomycosis;

oglufanide (IM-862, Implicit Bioscience), a two amino acid peptide, and variants and derivatives thereof, which can be used in the conjugates, therapeutic delivery systems, and compositions described herein to treat a microbial disorder or viral disorder such as hepatitis C virus infection;

omiganan (CPI-226, Omigard™, Migenix Inc.), a twelve amino acid peptide, and variants and derivatives thereof, which can be used in the conjugates, therapeutic delivery systems, and compositions described herein to treat a microbial disorder or viral disorder such as catheter infection and rosacea;

OP-145 (OctoPlus NV), a peptide, and variants and derivatives thereof, which can be used in the conjugates, therapeutic delivery systems, and compositions described herein to treat a microbial disorder or viral disorder such as otitis;

p-1025 (Sinclair Pharma plc), a nineteen amino acid peptide, and variants and derivatives thereof, which can be used in the conjugates, therapeutic delivery systems, and compositions described herein to treat a microbial disorder or viral disorder such as dental caries;

P-113 (PAC-113, HistaWash™, Histat Gingivitis Gel™, Histat Periodontal Wafer™, Pacgen Biopharmaceuticals Corp.), a twelve amino acid peptide, and variants and derivatives thereof, which can be used in the conjugates, therapeutic delivery systems, and compositions described herein to treat a microbial disorder or viral disorder such as Candida albicans infection and gingivitis;

Pep-F (5K, Milkhaus Laboratory Inc.), a peptide, and variants and derivatives thereof, which can be used in the conjugates, therapeutic delivery systems, and compositions described herein to treat a microbial disorder or viral disorder such as herpesvirus infection;

R-15-K (BlockAide/CR™, Adventrx Pharmaceuticals Inc.), a fifteen amino acid peptide, and variants and derivatives thereof, which can be used in the conjugates, therapeutic delivery systems, and compositions described herein to treat a microbial disorder or viral disorder such as HIV infection;

sifuvirtide (FusoGen Pharmaceuticals Inc.), a thirty-six amino acid peptide, and variants and derivatives thereof, which can be used in the conjugates, therapeutic delivery systems, and compositions described herein to treat a microbial disorder or viral disorder such as HIV infection;

SPC-3 (Columbia Laboratories), a polymeric fifty-six amino acid peptide, and variants and derivatives thereof, which can be used in the conjugates, therapeutic delivery systems, and compositions described herein to treat a microbial disorder or viral disorder such as HIV infection;

thymalfasin (Zadaxin™, Timosa™, Thymalfasin™, SciClone Pharmaceuticals), a twenty-eight amino acid peptide, and variants and derivatives thereof, which can be used in the conjugates, therapeutic delivery systems, and compositions described herein to treat a microbial disorder or viral disorder such as cancer (e.g., heptocellular carcinoma), hepatitis B virus infection, hepatitis C virus infection, HIV infection, influenza virus infection, aspergillus infection, and wound healing;

thymonoctan (FCE-25388, Pfizer), an eight amino acid peptide, and variants and derivatives thereof, which can be used in the conjugates, therapeutic delivery systems, and compositions described herein to treat a microbial disorder or viral disorder such as hepatitis virus infection and HIV infection:

thymopentin (TP-5, Timunox™, Johnson & Johnson), a five amino acid peptide, and variants and derivatives thereof, which can be used in the conjugates, therapeutic delivery systems, and compositions described herein to treat a microbial disorder or viral disorder such as lung infection and HIV infection;

tifuvirtide (R-724, T-1249, Roche), a thirty-nine amino acid peptide, and variants and derivatives thereof, which can be used in the conjugates, therapeutic delivery systems, and compositions described herein to treat a microbial disorder or viral disorder such as HIV infection;

TRI-1144 (Trimeris Inc.), a thirty-eight amino acid peptide, and variants and derivatives thereof, which can be used in the conjugates, therapeutic delivery systems, and compositions described herein to treat a microbial disorder or viral disorder such as HIV infection;

VIR-576 (Pharis Biotec), a forty amino acid peptide, and variants and derivatives thereof, which can be used in the conjugates, therapeutic delivery systems, and compositions described herein to treat a microbial disorder or viral disorder such as HIV infection; and

XOMA-629 (XOMA Ltd.), a fifteen amino acid peptide, and variants and derivatives thereof, which can be used in the conjugates, therapeutic delivery systems, and compositions described herein to treat a microbial disorder or viral disorder such as acne, Staphylococcus aureus infection, and impetigo.

In some embodiments, the agent is a derivative of a therapeutic peptide with pharmaceutical activity, such as an acetylated derivative or a pharmaceutically acceptable salt. In some embodiments, the therapeutic peptide is a prodrug such as a hexanoate conjugate.

Therapeutic peptide may mean a combination of therapeutic peptides that have been combined and attached to a polymer and/or loaded into the particle. Any combination of therapeutic peptides may be used. In certain embodiments for treating cancer, at least two traditional chemotherapeutic therapeutic peptides are attached to a polymer and/or loaded into the particle.

Additional therapeutic peptides compatible with therapeutic delivery systems, particles, and conjugates described herein include pituitary hormones (e.g., hGH), ANF, growth factors, e.g., growth factor releasing factor (GFRF), bMSH, somatostatin, platelet-derived growth factor releasing factor, human chorionic gonadotropin (hCG), erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, and polypeptides and polypeptide analogs and derivatives thereof.

Exemplary CDP-Therapeutic Peptide Conjugates

CDP-therapeutic peptide conjugates can be made using many different combinations of components described herein. For example, various combinations of cyclodextrins (e.g., beta-cyclodextrin), comonomers (e.g., PEG containing comonomers), linkers linking the cyclodextrins and comonomers, and/or linkers tethering the therapeutic peptide to the CDP are described herein.

A CDP-therapeutic peptide conjugate may be represented by the following formula:

CDP-CO-ABX-TP

In this formula,
CDP is the cyclodextrin-containing polymer shown below:

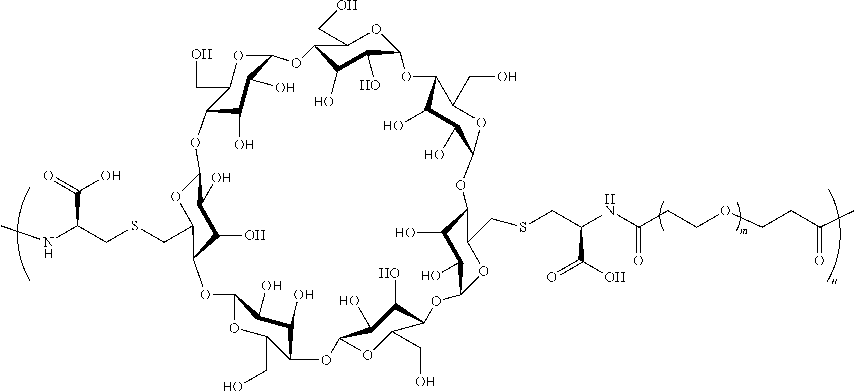

wherein the group

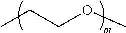

has a Mw of 3.4 kDa or less and n is at least 4, 5, 6, 7, 8, 9, 10, 11, 12, 13, 14, 15, 16, 17, 18, 19 or 20. Note that the therapeutic peptide is conjugated to the CDP through the carboxylic acid moieties of the polymer as provided above. Full loading of the therapeutic peptide onto the CDP is not required. In some embodiments, at least one, e.g., at least 2, 3, 4, 5, 6 or 7, of the carboxylic acid moieties remains unreacted with the therapeutic peptide after conjugation (e.g., a plurality of the carboxylic acid moieties remain unreacted).

CO represents the carbonyl group of the cysteine residue of the CDP;

A and B represent the link between the CDP and the therapeutic peptide. Position A is either a bond between linker B and the cysteine acid carbonyl of CDP, a bond between the therapeutic peptide and the cysteine acid carbonyl of CDP or depicts a portion of the linker that is attached via a bond to the cysteine acid carbonyl of the CDP. Position B is either not occupied or represents the linker or the portion of the linker that is attached via a bond to the therapeutic peptide;

X represents the heteroatom to which the linker is coupled on the therapeutic peptide; and

TP represents the therapeutic peptide.

One or more protecting groups can be used in the processes described above to make the CDP-therapeutic peptide conjugates described herein. A protecting group can be used to control the point of attachment of the therapeutic peptide and/or therapeutic peptide linker to position A. In some embodiments, the protecting group is removed and, in other embodiments, the protecting group is not removed. If a protecting group is not removed, then it can be selected so that it is removed in vivo (e.g., acting as a prodrug). An example is hexanoic acid which has been shown to be removed by lipases in vivo if used to protect a hydroxyl group in doxorubicin. Protecting groups are generally selected for both the reactive groups of the therapeutic peptide and the reactive groups of the linker that are not targeted to be part of the coupling reaction. The protecting group should be removable under conditions which will not degrade the therapeutic peptide and/or linker material. Examples include t-butyldimethylsilyl (“TBDMS”) and TROC (derived from 2,2,2-trichloroethoxy chloroformate). Carboxybenzyl (“CBz”) can also be used in place of TROC if there is selectivity seen for removal over olefin reduction. This can be addressed by using a group which is more readily removed by hydrogenation such as -methoxybenzyl OCO—. Other protecting groups may also be acceptable. One of skill in the art can select suitable protecting groups for the products and methods described herein.

### CDPs, Methods of Making Same, and Methods of Conjugating CDPs to Therapeutic Peptides

Generally, the CDP-therapeutic peptide conjugates described herein can be prepared in one of two ways: monomers bearing therapeutic peptides, targeting ligands, and/or cyclodextrin moieties can be polymerized, or polymer backbones can be derivatized with therapeutic peptides, targeting ligands, and/or cyclodextrin moieties.

Thus, in one embodiment, the synthesis of the CDP-therapeutic peptide conjugates can be accomplished by reacting monomers M-L-CD and M-L-D (and, optionally, M-L-T), wherein

CD represents a cyclic moiety, such as a cyclodextrin molecule, or derivative thereof;

L, independently for each occurrence, may be absent or represents a linker group;

D, independently for each occurrence, represents the same or different therapeutic peptide or prodrug thereof;

T, independently for each occurrence, represents the same or different targeting ligand or precursor thereof; and

M represents a monomer subunit bearing one or more reactive moieties capable of undergoing a polymerization reaction with one or more other M in the monomers in the reaction mixture, under conditions that cause polymerization of the monomers to take place.

In some embodiments, one or more of the therapeutic peptide moieties in the CDP-therapeutic peptide conjugate can be replaced with another therapeutic agent, e.g., another anticancer agent or anti-inflammatory agent.

In certain embodiments, the reaction mixture may further comprise monomers that do not bear CD, T, or D moieties, e.g., to space the derivatized monomer units throughout the polymer.

In an alternative embodiment, the invention contemplates synthesizing a CDP-therapeutic peptide conjugate by reacting a polymer P (the polymer bearing a plurality of reactive groups, such as carboxylic acids, alcohols, thiols, amines, epoxides, etc.) with grafting agents X-L-CD and/or Y-L-D (and, optionally, Z-L-T), wherein

CD represents a cyclic moiety, such as a cyclodextrin molecule, or derivative thereof;

L, independently for each occurrence, may be absent or represents a linker group;

D, independently for each occurrence, represents the same or different therapeutic peptide or prodrug thereof;

T, independently for each occurrence, represents the same or different targeting ligand or precursor thereof;

X, independently for each occurrence, represents a reactive group, such as carboxylic acids, alcohols, thiols, amines, epoxides, etc., capable of forming a covalent bond with a reactive group of the polymer; and

Y and Z, independently for each occurrence, represent inclusion hosts or reactive groups, such as carboxylic acids, alcohols, thiols, amines, epoxides, etc., capable of forming a covalent bond with a reactive group of the polymer or inclusion complexes with CD moieties grafted to the polymer, under conditions that cause the grafting agents to form covalent bonds and/or inclusion complexes, as appropriate, with the polymer or moieties grafted to the polymer.

In some embodiments, one or more of the therapeutic peptide moieties in the CDP-therapeutic peptide conjugate can be replaced with another therapeutic agent, e.g., another anticancer agent or anti-inflammatory agent.

For example, if the CDP includes alcohols, thiols, or amines as reactive groups, the grafting agents may include reactive groups that react with them, such as isocyanates, isothiocyanates, acid chlorides, acid anhydrides, epoxides, ketenes, sulfonyl chlorides, activated carboxylic acids (e.g., carboxylic acids treated with an activating agent such as PyBrOP, carbonyldiimidazole, or another reagent that reacts with a carboxylic acid to form a moiety susceptible to nucleophilic attack), or other electrophilic moieties known to those of skill in the art. In certain embodiments, a catalyst may be needed to cause the reaction to take place (e.g., a Lewis acid, a transition metal catalyst, an amine base, etc.) as will be understood by those of skill in the art.

In certain embodiments, the different grafting agents are reacted with the polymer simultaneously or substantially simultaneously (e.g., in a one-pot reaction), or are reacted sequentially with the polymer (optionally with a purification and/or wash step between reactions).

Another aspect of the present invention is a method for manufacturing the linear or branched CDPs and CDP-therapeutic peptide conjugates as described herein. While the discussion below focuses on the preparation of linear cyclodextrin molecules, one skilled in the art would readily recognize that the methods described can be adapted for producing branched polymers by choosing an appropriate comonomer precursor.

Accordingly, one embodiment of the invention is a method of preparing a linear CDP. According to the invention, a linear CDP may be prepared by copolymerizing a cyclodextrin monomer precursor disubstituted with one or more appropriate leaving groups with a comonomer precursor capable of displacing the leaving groups. The leaving group, which may be the same or different, may be any leaving group known in the art which may be displaced upon copolymerization with a comonomer precursor. In a preferred embodiment, a linear CDP may be prepared by iodinating a cyclodextrin monomer precursor to form a diiodinated cyclodextrin monomer precursor and copolymerizing the diiodinated cyclodextrin monomer precursor with a comonomer precursor to form a linear CDP having a repeating unit of formula I or IL provided in the section entitles “CDP-Therapeutic peptide conjugates” or a combination thereof, each as described above. In some embodiments, the cyclodextrin moiety precursors are in a composition, the composition being substantially free of cyclodextrin moieties having other than two positions modified to bear a reactive site (e.g. 1, 3, 4, 5, 6, or 7). While examples presented below discuss iodinated cyclodextrin moieties, one skilled in the art would readily recognize that the present invention contemplates and encompasses cyclodextrin moieties wherein other leaving groups such as alkyl and aryl sulfonate may be present instead of iodo groups. In a preferred embodiment, a method of preparing a linear cyclodextrin copolymer of the invention by iodinating a cyclodextrin monomer precursor as described above to form a diiodinated cyclodextrin monomer precursor of formula IVa, IVb, IVc or a mixture thereof:

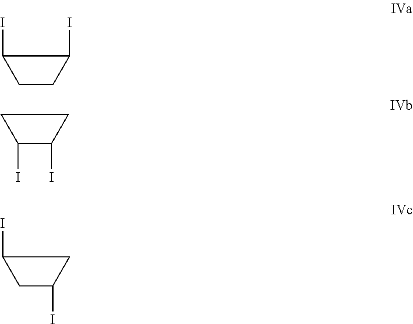

In some embodiments, the iodine moieties as shown on the cyclodextrin moieties are positioned such that the derivatization on the cyclodextrin is on the A and D glucopyranose moieties. In some embodiments, the iodine moieties as shown on the cyclodextrin moieties are positioned in such that the derivatization on the cyclodextrin is on the A and C glucopyranose moieties. In some embodiments, the iodine moieties as shown on the cyclodextrin moieties are positioned in such that the derivatization on the cyclodextrin is on the A and F glucopyranose moieties. In some embodiments, the iodine moieties as shown on the cyclodextrin moieties are positioned in such that the derivatization on the cyclodextrin is on the A and E glucopyranose moieties.

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also according to the invention, the iodo groups or other appropriate leaving group may be displaced with a group that permits reaction with a comonomer precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

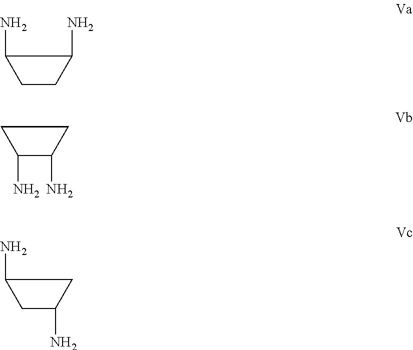

In some embodiments, the amino moieties as shown on the cyclodextrin moieties are positioned such that the derivatization on the cyclodextrin is on the A and D glucopyranose moieties. In some embodiments, the amino moieties as shown on the cyclodextrin moieties are positioned in such that the derivatization on the cyclodextrin is on the A and C glucopyranose moieties. In some embodiments, the amino moieties as shown on the cyclodextrin moieties are positioned in such that the derivatization on the cyclodextrin is on the A and F glucopyranose moieties. In some embodiments, the amino moieties as shown on the cyclodextrin moieties are positioned in such that the derivatization on the cyclodextrin is on the A and E glucopyranose moieties.

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-β-cyclodextrin). The cyclodextrin monomer precursor is aminated at only two positions. The diaminated cyclodextrin monomer precursor may then be copolymerized with a comonomer precursor, as described above, to produce a linear cyclodextrin copolymer having a repeating unit of formula I-II provided in the section entitles “CDP-Therapeutic peptide conjugates” or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

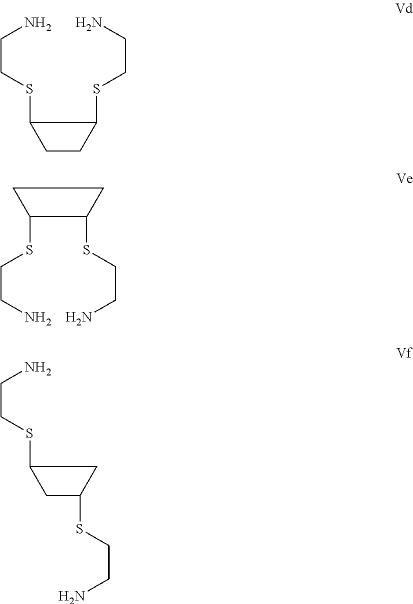

In some embodiments, the —SCH₂CH₂NH₂ moieties as shown on the cyclodextrin moieties are positioned such that the derivatization on the cyclodextrin is on the A and D glucopyranose moieties. In some embodiments, the —SCH₂CH₂NH₂ moieties as shown on the cyclodextrin moieties are positioned in such that the derivatization on the cyclodextrin is on the A and C glucopyranose moieties. In some embodiments, the —SCH₂CH₂NH₂ moieties as shown on the cyclodextrin moieties are positioned in such that the derivatization on the cyclodextrin is on the A and F glucopyranose moieties. In some embodiments, the —SCH₂CH₂NH₂ moieties as shown on the cyclodextrin moieties are positioned in such that the derivatization on the cyclodextrin is on the A and E glucopyranose moieties.

A linear oxidized CDP may also be prepared by oxidizing a reduced linear cyclodextrin-containing copolymer as described below. This method may be performed as long as the comonomer does not contain an oxidation sensitive moiety or group such as, for example, a thiol.

A linear CDP of the invention may be oxidized so as to introduce at least one oxidized cyclodextrin monomer into the copolymer such that the oxidized cyclodextrin monomer is an integral part of the polymer backbone. A linear CDP which contains at least one oxidized cyclodextrin monomer is defined as a linear oxidized cyclodextrin copolymer or a linear oxidized cyclodextrin-containing polymer. The cyclodextrin monomer may be oxidized on either the secondary or primary hydroxyl side of the cyclodextrin moiety. If more than one oxidized cyclodextrin monomer is present in a linear oxidized cyclodextrin copolymer of the invention, the same or different cyclodextrin monomers oxidized on either the primary hydroxyl side, the secondary hydroxyl side, or both may be present. For illustration purposes, a linear oxidized cyclodextrin copolymer with oxidized secondary hydroxyl groups has, for example, at least one unit of formula VIa or VIb:

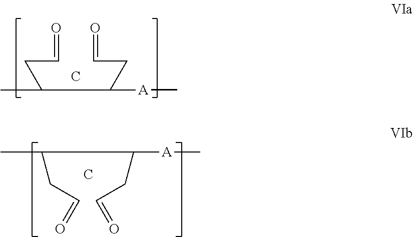

In formulae VIa and VIb, C is a substituted or unsubstituted oxidized cyclodextrin monomer and the comonomer (i.e., shown herein as A) is a comonomer bound, i.e., covalently bound, to the oxidized cyclodextrin C. Also in formulae VIa and VIb, oxidation of the secondary hydroxyl groups leads to ring opening of the cyclodextrin moiety and the formation of aldehyde groups.

A linear oxidized CDP copolymer may be prepared by oxidation of a linear cyclodextrin copolymer as discussed above. Oxidation of a linear cyclodextrin copolymer of the invention may be accomplished by oxidation techniques known in the art. (Hisamatsu et al., Starch 44:188-191 (1992)). Preferably, an oxidant such as, for example, sodium periodate is used. It would be understood by one of ordinary skill in the art that under standard oxidation conditions that the degree of oxidation may vary or be varied per copolymer. Thus in one embodiment of the invention, a CDP may contain one oxidized cyclodextrin monomer. In another embodiment, substantially all cyclodextrin monomers of the copolymer would be oxidized.

Another method of preparing a linear oxidized CDP involves the oxidation of a diiodinated or diaminated cyclodextrin monomer precursor, as described above, to form an oxidized diiodinated or diaminated cyclodextrin monomer precursor and copolymerization of the oxidized diiodinated or diaminated cyclodextrin monomer precursor with a comonomer precursor. In a preferred embodiment, an oxidized diiodinated cyclodextrin monomer precursor of formula VIIa, VIIb, VIIc, or a mixture thereof:

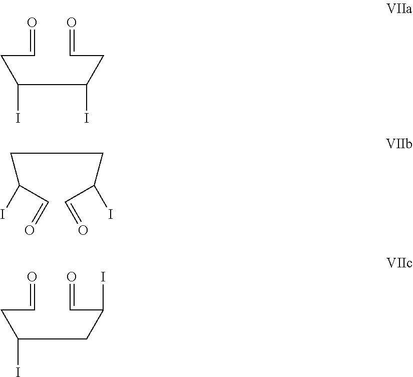

may be prepared by oxidation of a diiodinated cyclodextrin monomer precursor of formulae IVa, IVb, IVc, or a mixture thereof, as described above. In another preferred embodiment, an oxidized diaminated cyclodextrin monomer precursor of formula VIIIa, VIIIb, VIIIc or a mixture thereof:

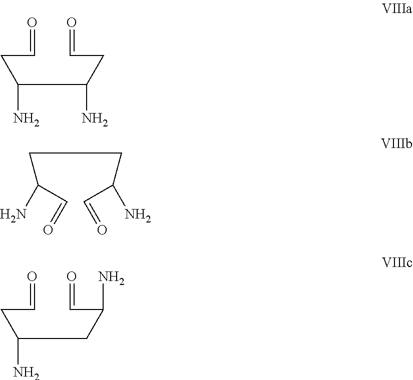

may be prepared by amination of an oxidized diiodinated cyclodextrin monomer precursor of formulae VIIa, VIIb, VIIc, or a mixture thereof, as described above. In still another preferred embodiment, an oxidized diaminated cyclodextrin monomer precursor of formula IXa, IXb, IXc or a mixture thereof:

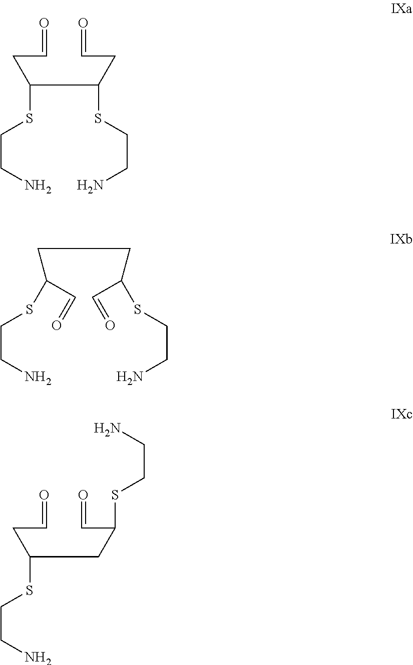 [SMILES: NCCSC(CC=O)C(CC=O)SCCN ; NCCSC(C=O)CCC(C=O)SCCN ; NCCSC(C=O)CC(CC=O)SCCN]

may be prepared by displacement of the iodo or other appropriate leaving groups of an oxidized cyclodextrin monomer precursor disubstituted with an iodo or other appropriate leaving group with the amino or other nucleophilic group containing moiety such as, e.g. HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine.

Alternatively, an oxidized diiodinated or diaminated cyclodextrin monomer precursor, as described above, may be prepared by oxidizing a cyclodextrin monomer precursor to form an oxidized cyclodextrin monomer precursor and then diiodinating and/or diaminating the oxidized cyclodextrin monomer, as described above. As discussed above, the cyclodextrin moiety may be modified with other leaving groups other than iodo groups and other amino group containing functionalities. The oxidized diiodinated or diaminated cyclodextrin monomer precursor may then be copolymerized with a comonomer precursor, as described above, to form a linear oxidized cyclodextrin copolymer of the invention.

A linear oxidized CDP may also be further modified by attachment of at least one ligand to the copolymer. The ligand is as described above.

In some embodiments, a CDP comprises: cyclodextrin moieties, and comonomers which do not contain cyclodextrin moieties (comonomers), and wherein the CDP comprises at least four, five six, seven, eight, nine, ten, eleven, twelve, thirteen, fourteen, fifteen, sixteen, seventeen, eighteen, nineteen or twenty cyclodextrin moieties and at least four, five six, seven, eight, nine, ten, eleven, twelve, thirteen, fourteen, fifteen, sixteen, seventeen, eighteen, nineteen or twenty comonomers.

In some embodiments, the at least four, five six, seven, eight, etc., cyclodextrin moieties and at least four, five six, seven, eight, nine, ten, eleven, twelve, thirteen, fourteen, fifteen, sixteen, seventeen, eighteen, nineteen or twenty comonomers alternate in the water soluble linear polymer.

In some embodiments, the cyclodextrin moieties comprise linkers to which therapeutic agents may be further linked.

In some embodiments, the CDP has no therapeutic peptides attached. In some embodiments, the CDP has a plurality (i.e., more than one) of therapeutic peptides attached (e.g., through a linker). In some embodiments, the therapeutic peptides are attached via a second linker.

In some embodiments, the comonomer is a compound containing residues of least two functional groups through which reaction and thus linkage of the cyclodextrin monomers is achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the residues of the two functional groups are the same and are located at termini of the comonomer. In some embodiments, a comonomer contains one or more pendant groups with at least one functional group through which reaction and thus linkage of a therapeutic peptide can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient therapeutic peptide such that up to at least 5%, 10%, 15%, 20%, 25%, 30%, or even 35% by weight of the water soluble linear polymer, when conjugated, is therapeutic peptide.

In some embodiments, the molecular weight of the CDP is 10,000-500,000 Da, e.g., about 30,000 to about 100,000 Da.

In some embodiments, the cyclodextrin moieties make up at least about 2%, 5%, 10%, 11%, 12%, 13%, 14%, 15%, 16%, 17%, 18%, 19%, 20%, 30%, 50% or 80% of the polymer by weight.

In some embodiments, the CDP is made by a method comprising providing cyclodextrin moiety precursors modified to bear one reactive site at each of exactly two positions, and reacting the cyclodextrin moiety with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and comonomer is produced.

In some embodiments, the CDP comprises a comonomer selected from the group consisting of an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain.

In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP is a polymer of the following formula:

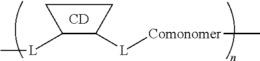

wherein each L is independently a linker, each comonomer is independently a comonomer described herein, and n is at least 4, 5, 6, 7, 8, 9, 10, 11, 12, 13, 14, 15, 16, 17, 18, 19 or 20. In some embodiments, the molecular weight of the comonomer is from about 2000 to about 5000 Da (e.g., from about 3000 to about 4000 Da (e.g., about 3400 Da).

In some embodiments, the CDP is a polymer of the following formula:

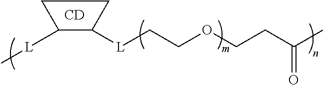

wherein each L is independently a linker, wherein the group

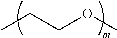

has a Mw of 3.4 kDa or less and n is at least 4, 5, 6, 7, 8, 9, 10, 11, 12, 13, 14, 15, 16, 17, 18, 19 or 20.

In some embodiments,

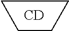

is alpha, beta or gamma cyclodextrin, e.g., beta cyclodextrin.

In some embodiments, each L independently comprises an amino acid or a derivative thereof. In some embodiments, at least one L comprises cysteine or a derivative thereof. In some embodiments, each L comprises cysteine. In some embodiments, each L is cysteine and the cysteine is connected to the CD by way of a thiol linkage.

In some embodiments, the CDP is a polymer of the following formula:

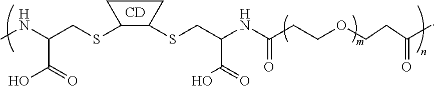

wherein the group

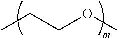

has a Mw of 3.4 kDa or less and n is at least 4, 5, 6, 7, 8, 9, 10, 11, 12, 13, 14, 15, 16, 17, 18, 19 or 20.

In some embodiments,

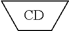

is alpha, beta or gamma cyclodextrin, e.g., beta cyclodextrin.

In some embodiments, the CDP is a polymer of the following formula:

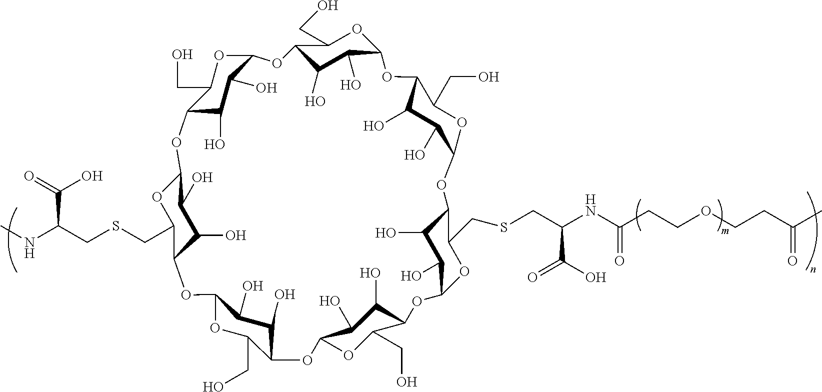

wherein the group

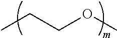

has a Mw of 3.4 kDa or less and n is at least 4, 5, 6, 7, 8, 9, 10, 11, 12, 13, 14, 15, 16, 17, 18, 19 or 20.

In some embodiments, the group

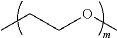

has a Mw of 3.4 kDa and the Mw of the compound as a whole is from 27 kDa to 99.6 kDa.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four, five six, seven, eight, or more, cyclodextrin moieties and at least four, five six, seven, eight, or more, comonomers.

In some embodiments, the at least four, five, six, seven, eight, or more cyclodextrin moieties and at least four, five, six, seven, eight, or more comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin comonomers comprise linkers to which therapeutic peptides may be further linked. In some embodiments, the therapeutic peptides are linked via second linkers.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—. —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient therapeutic peptide such that up to at least 3%, 5%, 10%, 15%, 20%, 25%, 30%, or even 35% by weight of the CDP, when conjugated, is therapeutic peptide.

In some embodiments, the CDP has a molecular weight of 10,000-500,000. In some embodiments, the cyclodextrin moieties make up at least about 2%, 5%, 10%, 20%, 30%, 50% or 80% of the CDP by weight.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain. the CDP comprises a comonomer selected from the group consisting of a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP of the following formula can be made by the scheme below:

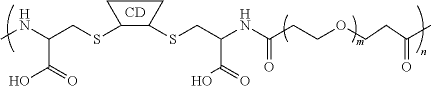

providing a compound of formula A and formula B:

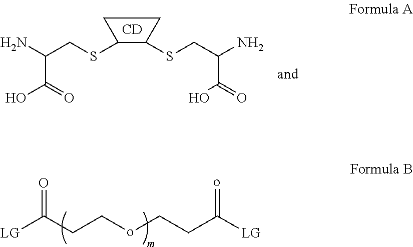

wherein LG is a leaving group;
and contacting the compounds under conditions that allow for the formation of a covalent bond between the compounds of formula A and B, to form a polymer of the following formula:

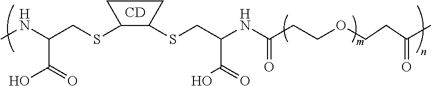

wherein the group

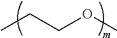

has a Mw of 3.4 kDa or less and n is at least four.

In some embodiments, Formula B is

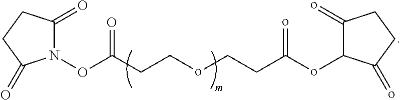

In some embodiments, the group

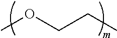

has a Mw of 3.4 kDa and the Mw of the compound is from 27 kDa to 99.6 kDa.

In some embodiments, the compounds of formula A and formula B are contacted in the presence of a base. In some embodiments, the base is an amine containing base. In some embodiments, the base is DEA.

In some embodiments, a CDP of the following formula can be made by the scheme below:

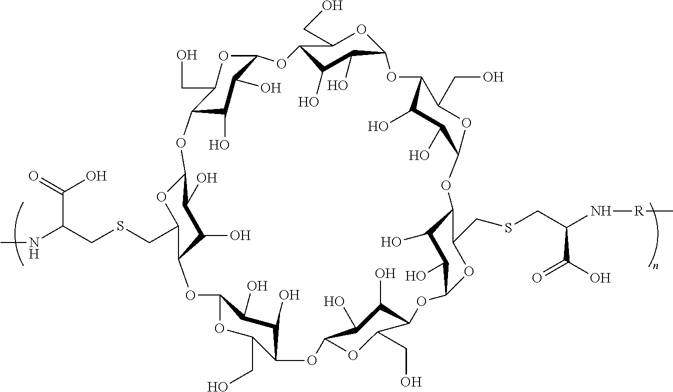

wherein R is of the form:

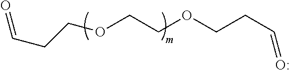

comprising the steps of:

reacting a compound of the formula below:

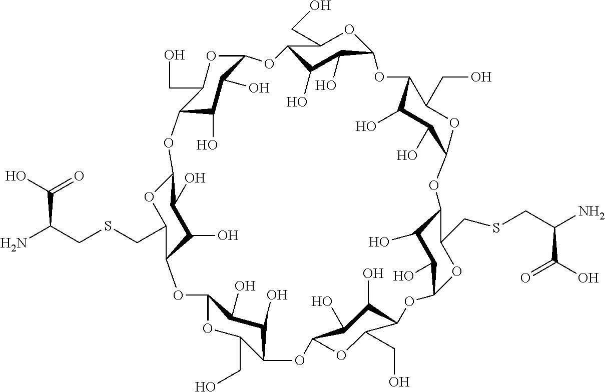 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

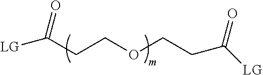

wherein the group

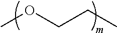

has a Mw of 3.4 kDa or less and n is at least four, in the presence of a non-nucleophilic organic base in a solvent.

In some embodiments,

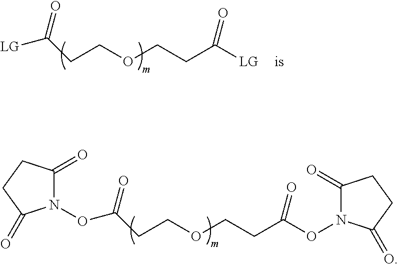

In some embodiments, the solvent is a polar aprotic solvent. In some embodiments, the solvent is DMSO.

In some embodiments, the method also includes the steps of dialysis; and lyophilization.

In some embodiments, a CDP provided below can be made by the following scheme:

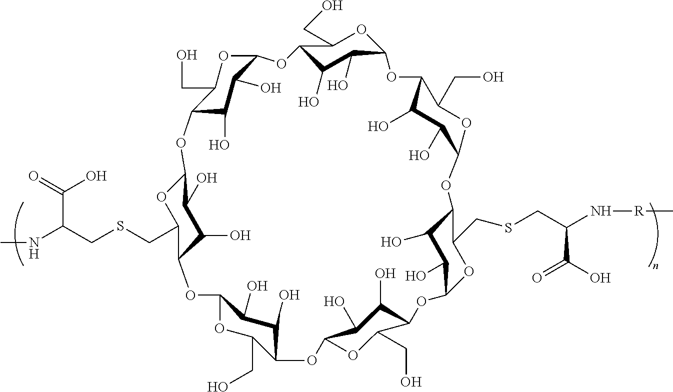

wherein R is of the form:

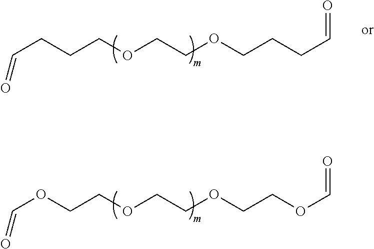

comprising the steps of:

reacting a compound of the formula below:

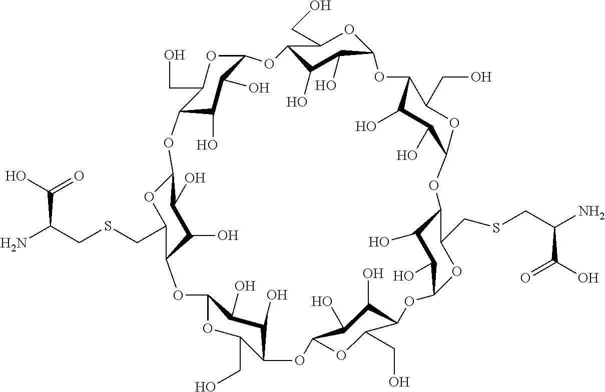 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

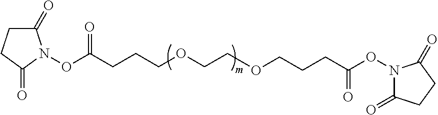

wherein the group

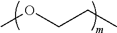

has a Mw of 3.4 kDa or less and n is at least four, or with a compound provided below:

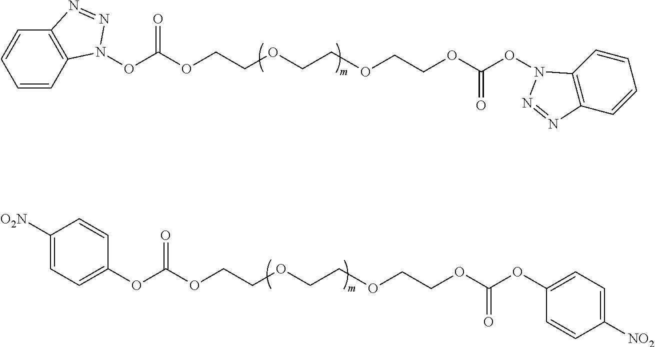

wherein the group

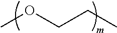

has a Mw of 3.4 kDa; in the presence of a non-nucleophilic organic base in DMSO;

and dialyzing and lyophilizing the following polymer

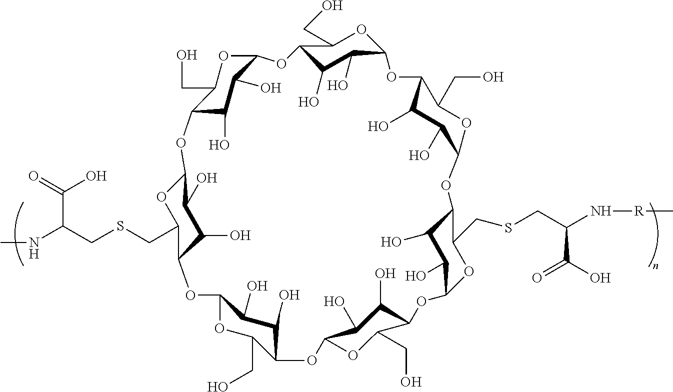

A CDP described herein may be attached to or grafted onto a substrate. The substrate may be any substrate as recognized by those of ordinary skill in the art. In another preferred embodiment of the invention, a CDP may be crosslinked to a polymer to form, respectively, a crosslinked cyclodextrin copolymer or a crosslinked oxidized cyclodextrin copolymer. The polymer may be any polymer capable of crosslinking with a CDP (e.g., polyethylene glycol (PEG) polymer, polyethylene polymer). The polymer may also be the same or different CDP. Thus; for example, a linear CDP may be crosslinked to any polymer including, but not limited to, itself, another linear CDP, and a linear oxidized CDP. A crosslinked linear CDP may be prepared by reacting a linear CDP with a polymer in the presence of a crosslinking agent. A crosslinked linear oxidized CDP may be prepared by reacting a linear oxidized CDP with a polymer in the presence of an appropriate crosslinking agent. The crosslinking agent may be any crosslinking agent known in the art. Examples of crosslinking agents include dihydrazides and disulfides. In a preferred embodiment, the crosslinking agent is a labile group such that a crosslinked copolymer may be uncrosslinked if desired.

A linear CDP and a linear oxidized CDP may be characterized by any means known in the art. Such characterization methods or techniques include, but are not limited to, gel permeation chromatography (GPC), matrix assisted laser desorption ionization-time of flight mass spectrometry (MALDI-TOF Mass spec), ¹H and ¹³C NMR, light scattering and titration.

The invention also provides a cyclodextrin composition containing at least one linear CDP and at least one linear oxidized CDP as described above. Accordingly, either or both of the linear CDP and linear oxidized CDP may be crosslinked to another polymer and/or bound to a ligand as described above. Therapeutic compositions according to the invention contain a therapeutic peptide and a linear CDP or a linear oxidized CDP, including crosslinked copolymers. A linear CDP, a linear oxidized CDP and their crosslinked derivatives are as described above. The therapeutic peptide may be any synthetic, semi-synthetic or naturally occurring biologically active therapeutic peptide, including those known in the art.

One aspect of the present invention contemplates attaching a therapeutic peptide to a CDP for delivery of a therapeutic peptide. The present invention discloses various types of linear, branched, or grafted CDPs wherein a therapeutic peptide is covalently bound to the polymer. In certain embodiments, the therapeutic peptide is covalently linked via a biohydrolyzable bond, for example, an ester, amide, carbamates, or carbonate.

A general strategy for synthesizing linear, branched or grafted cyclodextrin-containing polymers (CDPs) for loading a therapeutic peptide, and an optional targeting ligand is shown in Scheme II.

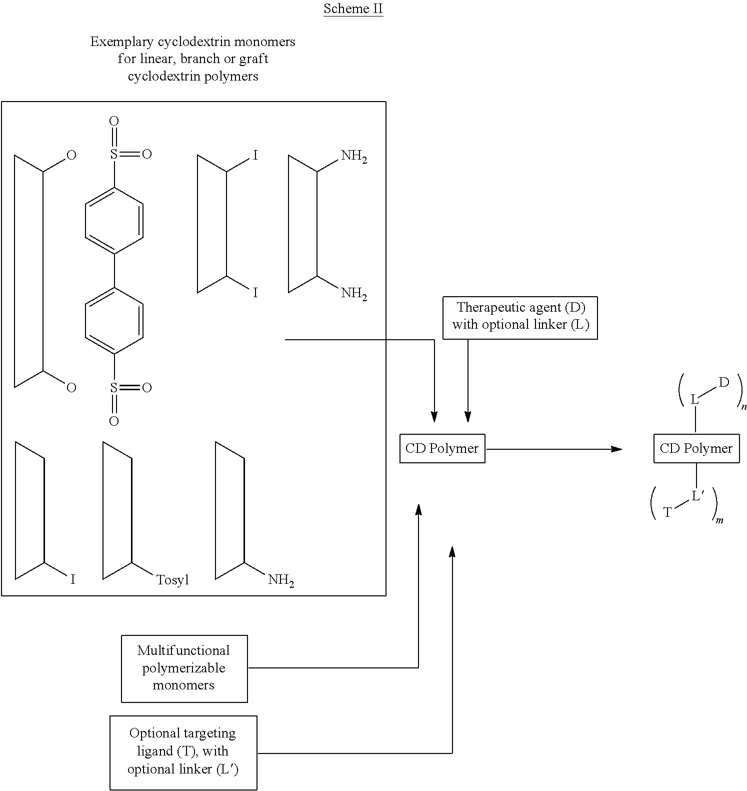

To illustrate further, comonomer precursors (shown in the scheme below as A), cyclodextrin moieties, therapeutic peptides, and/or targeting ligands may be assembled as shown in Schemes IIa-IIb below. Note that in schemes IIa-IIb, in any given reaction there may be more than one comonomer precursor, cyclodextrin moiety, therapeutic agent or targeting ligand that is of the same type or different. Furthermore, prior to polymerization, one or more comonomer precursor, cyclodextrin moiety, therapeutic agent or targeting ligand may be covalently linked with each other in one or more separate step. The scheme as provided above includes embodiments, where not all available positions for attachment of the therapeutic peptide are occupied on the CDP. For example, in some embodiments, less than all of the available points of attachments are reacted, leaving less than 100% yield of the therapeutic peptide onto the polymer. Accordingly, the loading of the therapeutic peptide onto the polymer can vary. This is also the case regarding a targeting agent when a targeting agent is included.

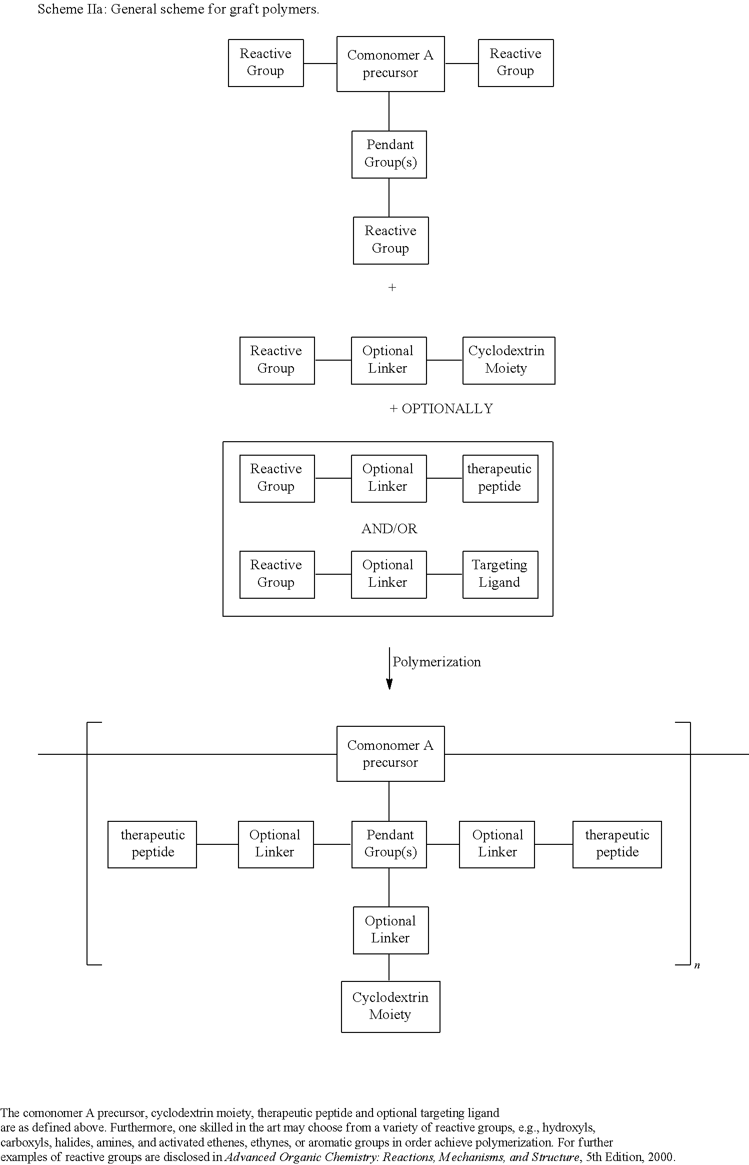

In some embodiments, one or more of the therapeutic peptide moieties in the CDP-therapeutic peptide conjugate can be replaced with another therapeutic agent, e.g., another anticancer agent or anti-inflammatory agent.

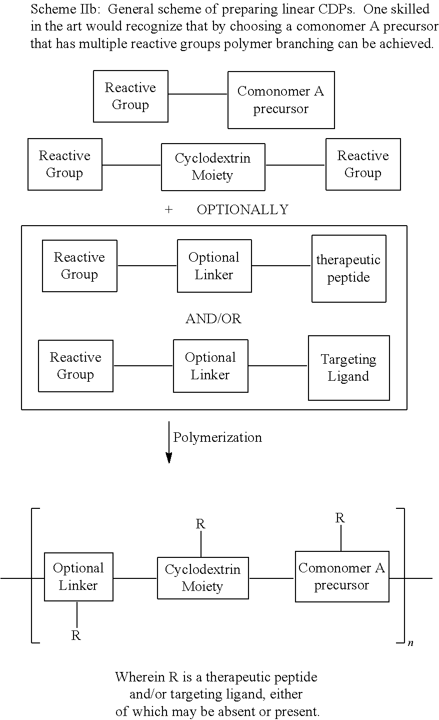

In some embodiments, one or more of the therapeutic peptide moieties in the CDP-therapeutic peptide conjugate can be replaced with another therapeutic agent, e.g., another anticancer agent or anti-inflammatory agent.

Examples of different ways of synthesizing CDP-therapeutic peptide conjugates are shown in Schemes III-VIII below. In each of Schemes III-VIII, one or more of the therapeutic peptide moieties in the CDP-therapeutic peptide conjugate can be replaced with another therapeutic agent, e.g., another anticancer agent or anti-inflammatory agent.

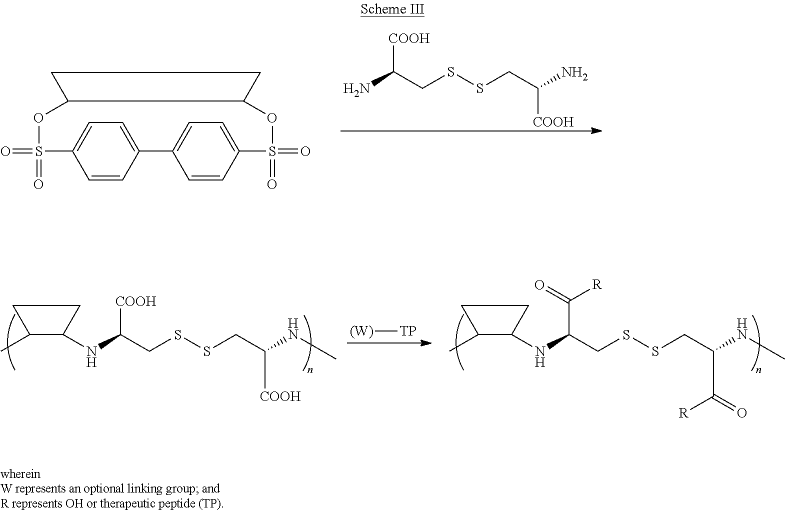

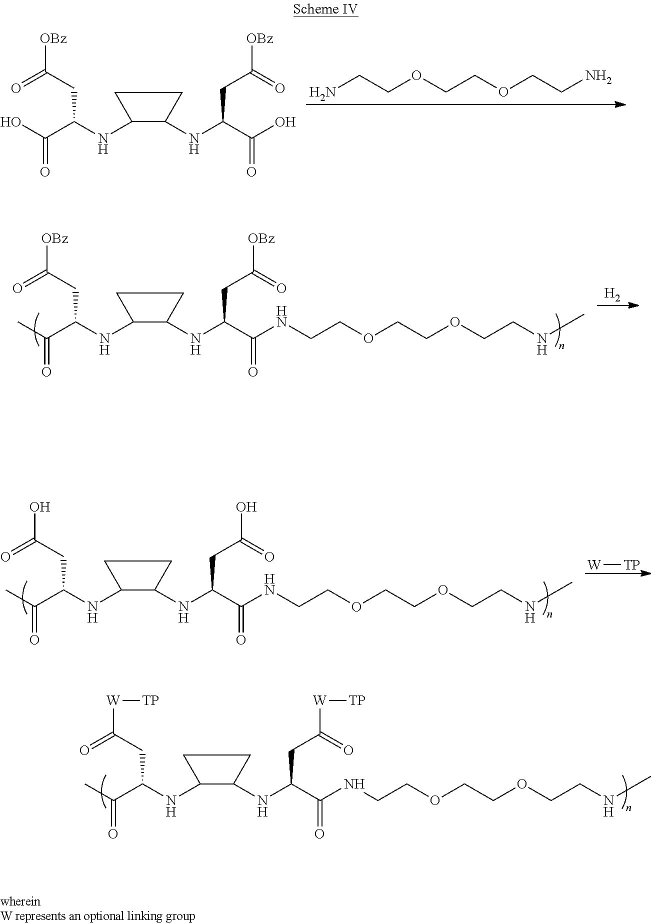

Scheme IV, as provided above, includes embodiments where W-therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

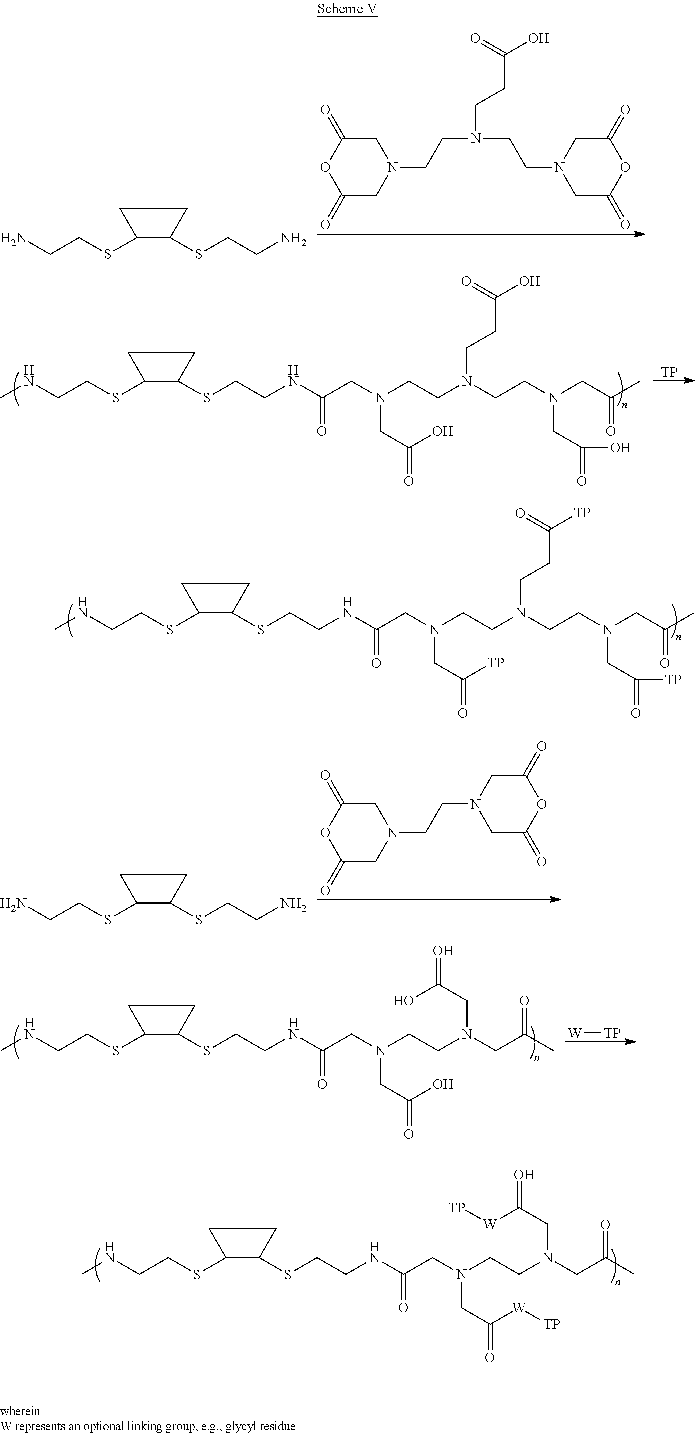

Scheme V, as provided above, includes embodiments where W-therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

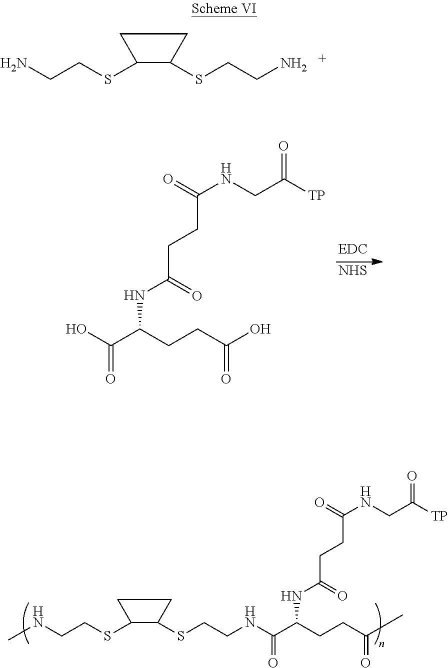

Scheme VI, as provided above, includes embodiments where therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

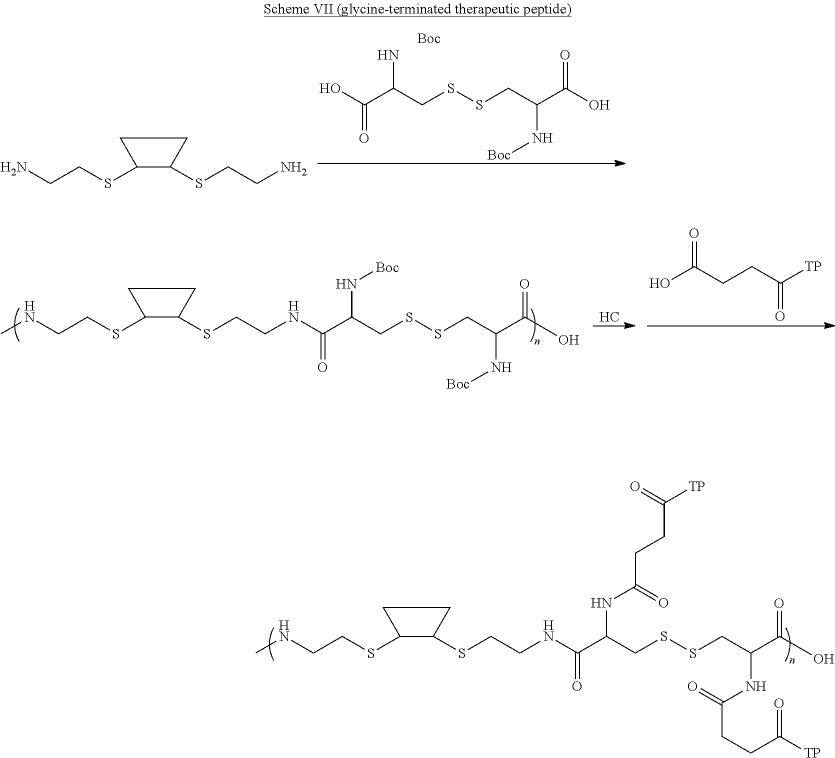

Scheme VII, as provided above, includes embodiments where a therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

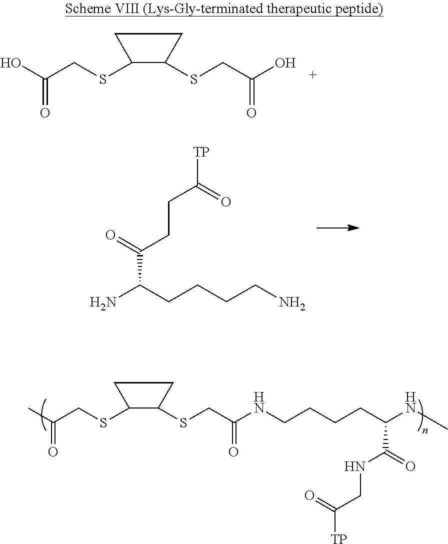

Scheme VIII, as provided above, includes embodiments where therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

Additional examples of methods of synthesizing CDP-therapeutic peptide conjugates are shown in Schemes IX-XIV below. In each of Schemes IX-XIV, one or more of the therapeutic peptide moieties in the CDP-therapeutic peptide conjugate can be replaced with another therapeutic agent, e.g., another anticancer agent or anti-inflammatory agent.

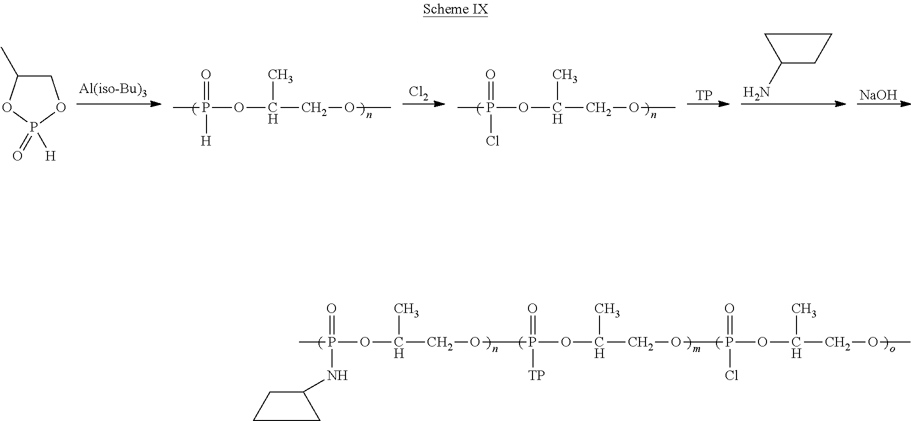

Scheme IX, as provided above, includes embodiments where therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

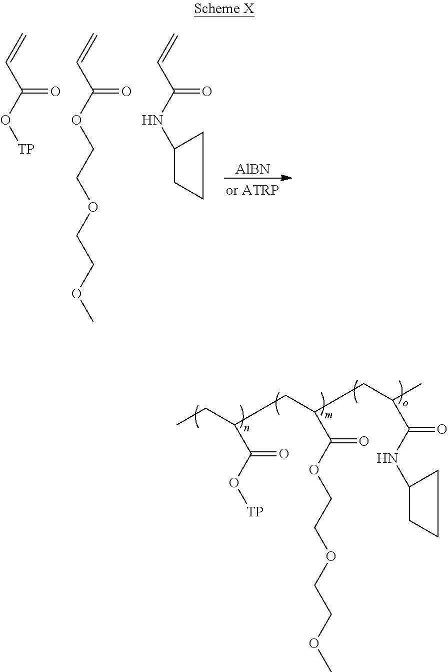

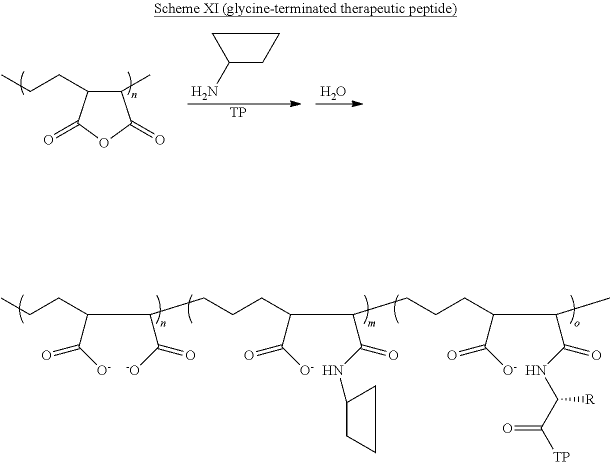

Scheme XI, as provided above, includes embodiments where gly-therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

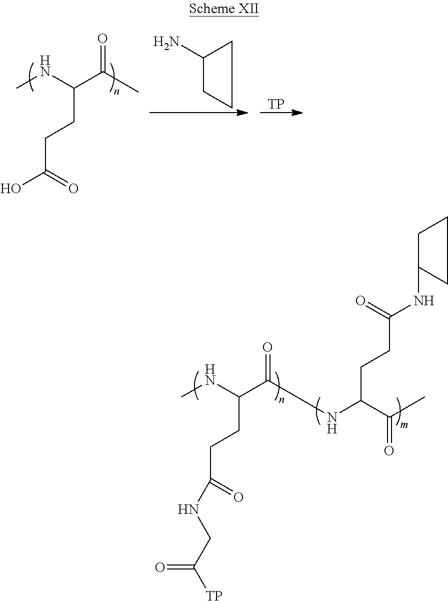

Scheme XII, as provided above, includes embodiments where therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

The present invention further contemplates CDPs and CDP-conjugates synthesized using CD-biscysteine monomer and a di-NHS ester such as PEG-DiSPA or PEG-BTC as shown in Schemes XIII-XIV below.

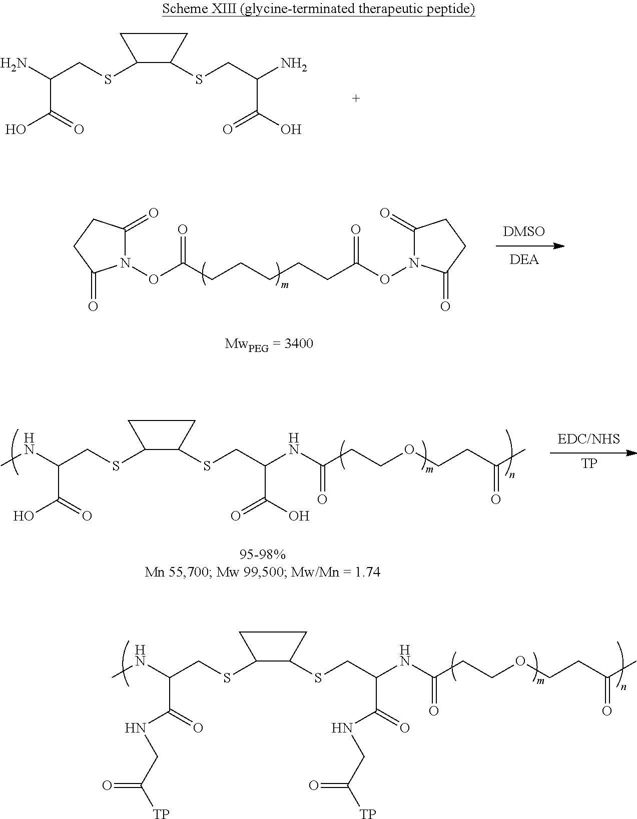

Scheme XIII, as provided above, includes embodiments where therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

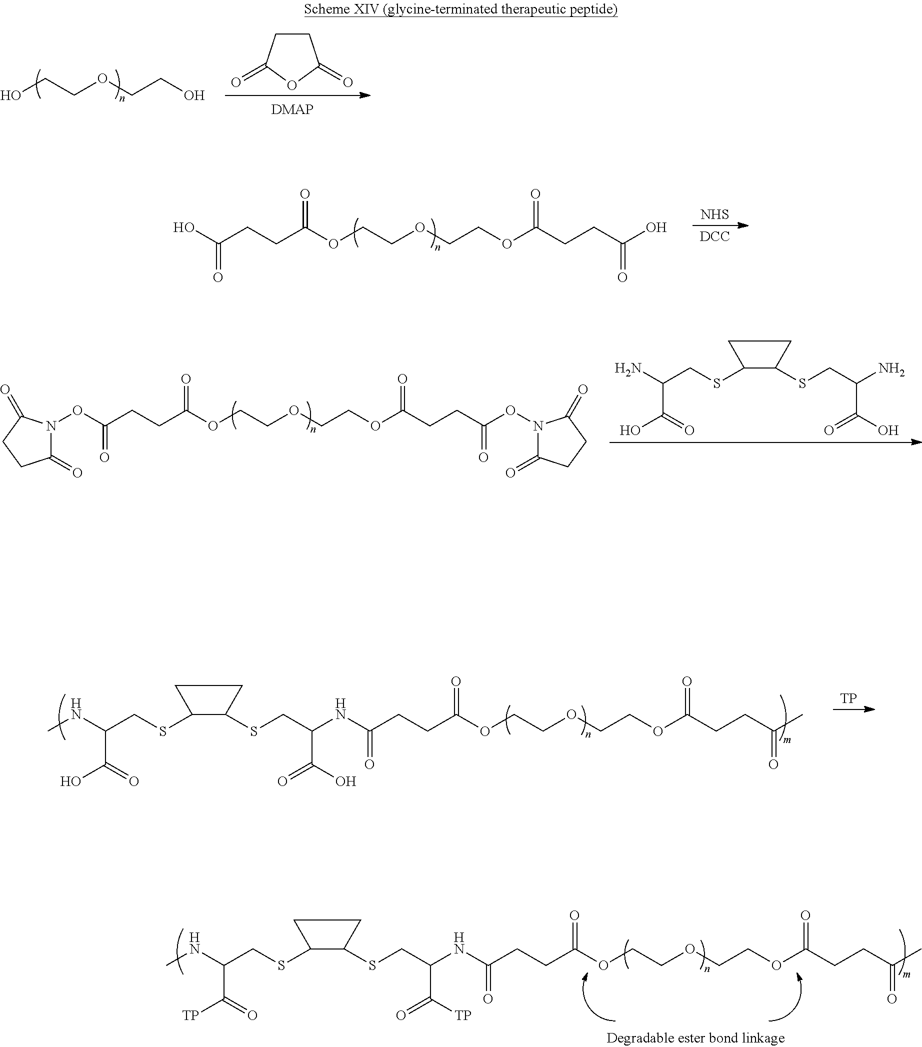

Scheme XIV, as provided above, includes embodiments where gly-therapeutic peptide is absent in one or more positions as provided above. This can be achieved, for example, when less than 100% yield is achieved when coupling the therapeutic peptide to the polymer and/or when less than an equivalent amount of therapeutic peptide is used in the reaction. Accordingly, the loading of the therapeutic peptide, by weight of the polymer, can vary.

### Exemplary CDP-Therapeutic Peptide Conjugates

CDP-therapeutic peptide conjugates can be made using many different combinations of components described herein. One or more peptide-polymer conjugates may be linked to CDP using the linker chemistry described herein.

Exemplary CDP-therapeutic peptide conjugates include the following.

1) CDP-Ester Linker-Therapeutic Peptide

This conjugate will generally include the modification of carbonyl end group of peptide with amino group which can be conjugated to the CDP polymer. This linker will have an ester bond to the therapeutic peptide which can be cleaved off at high pH or by an enzyme such as estearase. An exemplary scheme is shown below:

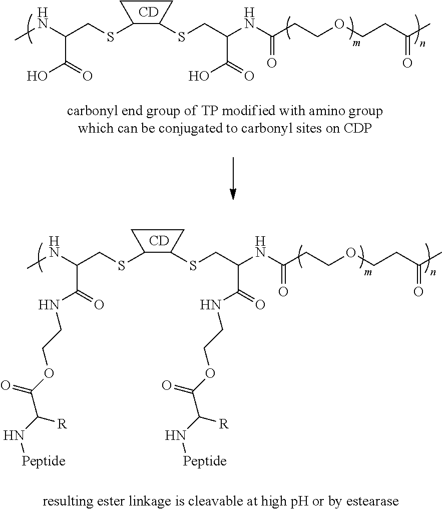

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

2) CDP-Amide Linker-Therapeutic Peptide

This conjugate will generally include the modification of carbonyl end group of CDP with an amine functional group. The amino group of CDP derivatives then can react with carbonyl end group of therapeutic peptide or carbonyl groups on the side chains of amino acids such as glutamic acid or aspartic acid to form a stable amide bond. An exemplary scheme is shown below.

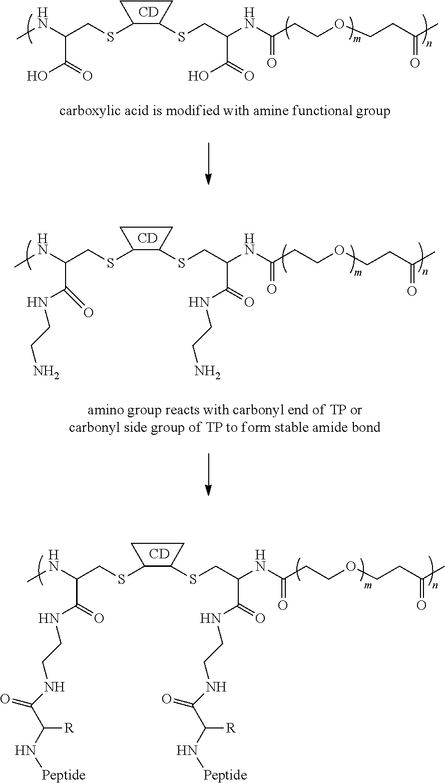

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

3) CDP-Disulfide Linker-Therapeutic Peptide

This conjugate will generally include the modification of carbonyl end group of CDP with a reactive sulfihydryl group. This group can react with therapeutic peptides containing cysteine groups which could be located at the end group or along the chain. It can also react with peptides that are derivatized with sulfihydryl group. The disulfide bond can be reduced internally to release peptide. An exemplary scheme is shown below.

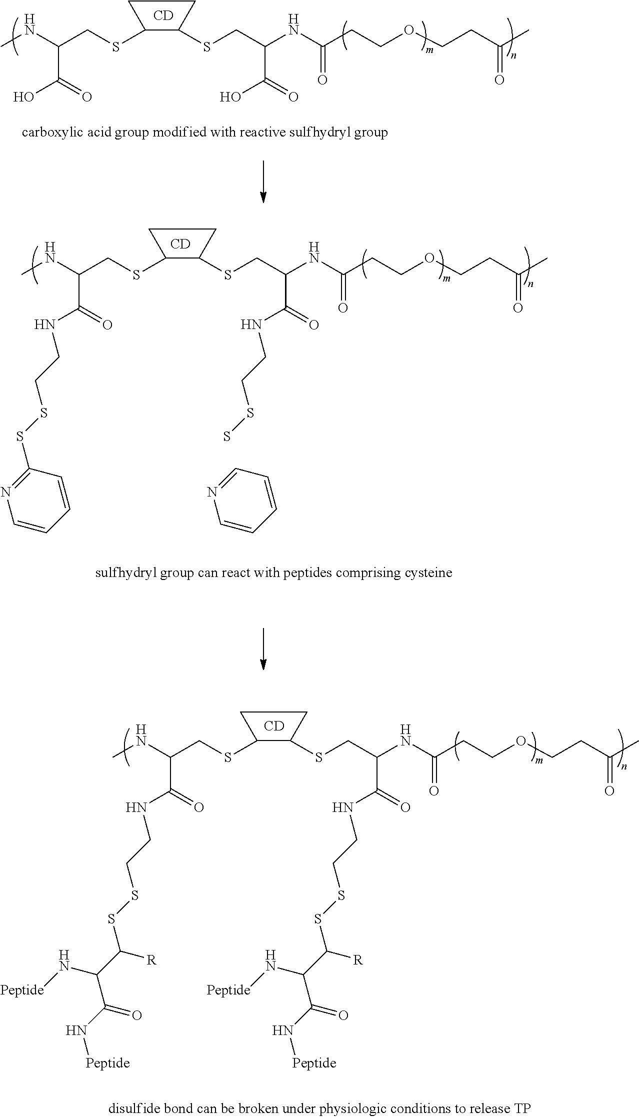

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

4) CDP-Disulfide Linker-Therapeutic Peptide

This conjugate will generally include the modification of hydroxyl group on tyrosine with disulfide amino group which can be conjugated to CDP. Upon reduction of disulfide bond, the linker will cyclize and kick out the polypeptides. Tyrosine or phenol group derivatized amino acids can be used. The disulfide bond can be reduced internally to release therapeutic peptide. An exemplary scheme is shown below.

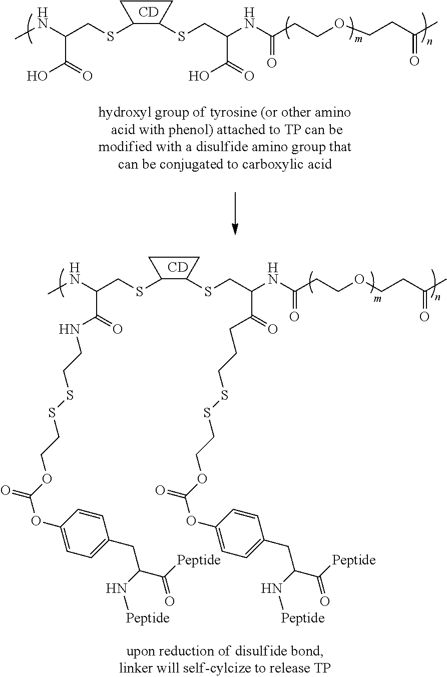

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

5) CDP-Thioether Linker-Therapeutic Peptide

This conjugate will generally include the modification of the carbonyl end group of CDP with a maleimide group. This group can react with therapeutic peptides containing cysteine located at the end group or along the peptide chain. It can also react with peptides that are derivatized with sulfihydryl group. This conjugate will have a non-releasing thioether bond. An exemplary scheme is shown below.

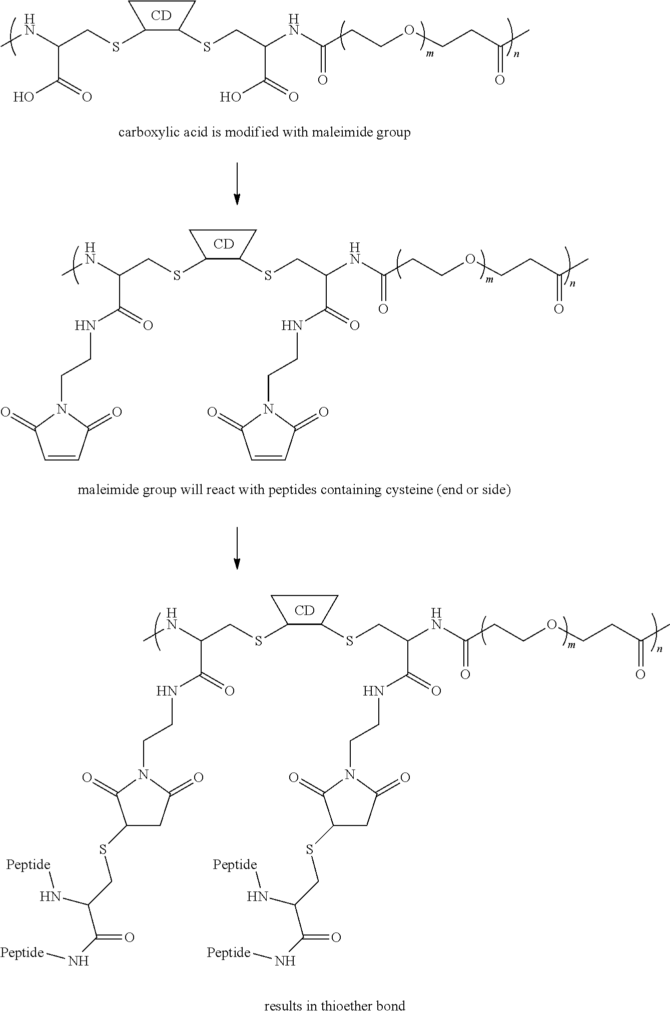

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

6) Linkers Synthesized Using Click Chemistry

A CDP polymer terminated with an alkyne group (e.g. acetylene) can be conjugated to a therapeutic peptide with an azide group, or a CDP polymer terminated with an azide group can be conjugated to a therapeutic peptide with an alkyne group. In order to be able to release the therapeutic peptide more easily, a cleavable linker (e.g. ester or disulfide) can be introduced in between the azide or alkyne functional group and the therapeutic peptide.

A CDP terminated with an acetylene group (alkyne) can be reacted, with an azide functional therapeutic peptide. The synthesis can include the use of an insoluble substrate, e.g., to functionalize the therapeutic peptide. In some embodiments, a terminal amino-functional group (e.g. glycine) can be converted into an alkyne moiety via a coupling reaction with 4-pentynoic acid in the presence of N,N″-dicyclohexylcarbodiimide. For example:

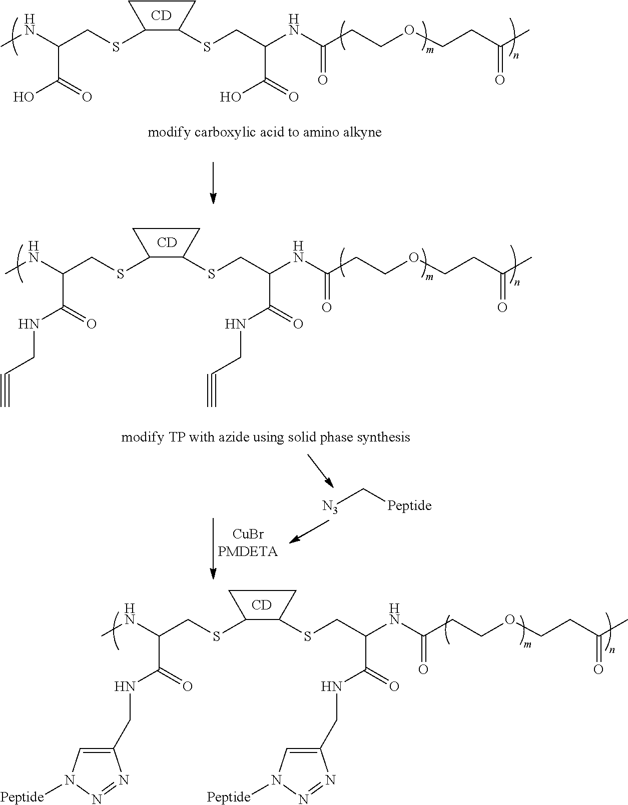

In some embodiments, the resulting conjugate may have less than full loading with therapeutic peptide, e.g., not all available carbonyl end groups will have an ester linkage to a therapeutic peptide. The loading may be less than about 50%, less than about 30%, less than about 25%, less than about 15%, less than about 10%, less than about 5%, less than about 1% weight of therapeutic peptide relative to the conjugate. In some embodiments, the TP-loaded CDP will comprise one or more subunits of the dual-loaded CD-PEG copolymer shown above.

Other exemplary coupling reactions using click chemistry include a Michael Addition (1,4 addition) (e.g., addition of a base (NaOH or KOH) to form an enolate, and allowing the enolate to react with an α,β-unsaturated ketone); Diels Alder reaction (e.g., reaction of a conjugated diene to an alkene group to form a cyclohexene group); and an epoxy ring opening with amine or hydroxyl groups (e.g., a nucleophilic substitution-Sn2 reaction).

### Particles

In embodiments, the CDP-therapeutic peptide conjugate forms or is provided as a particle (e.g., a nanoparticle). In some embodiments, the particle has a diameter of less than 500 nm, e.g., less than 300 nm (e.g., the particles in a composition described herein have a Dv90 of less than 500 nm, e.g., less than 300 nm). The nanoparticles generally range in size from 10 to 300 nm in diameter, e.g., 10 to 280, 20 to 280, 30 to 250, 30 to 200, 20 to 150, 30 to 100, 20 to 80, 10 to 80, 10 to 70, 20 to 60 or 20 to 50 nm 10 to 70, 10 to 60 or 10 to 50 nm diameter. In one embodiment, the nanoparticle is 20 to 60 nm in diameter. In one embodiment, the composition comprises a population or a plurality of nanoparticles with an average diameter from 10 to 300 nm, e.g., 20 to 280, 15 to 250, 15 to 200, 20 to 150, 15 to 100, 20 to 80, 15 to 80, 15 to 70, 15 to 60, 15 to 50, or 20 to 50 nm. In one embodiment, the average nanoparticle diameter is from 15 to 60 nm (e.g., 20-60 nm), e.g., the average of the nanoparticles in a composition described herein have a Dv90 of 15 to 60 nm. In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

Conjugate Number

Conjugate number, as used herein, is the number of cyclodextrin containing polymer (“CDP”) therapeutic agent (e.g., therapeutic peptide) conjugate molecules, present in a particle or nanoparticle. For purposes of determining conjugate number, a particle or nanoparticle is an entity having one, or typically, more than one CDP therapeutic agent conjugate molecules, which, at the concentration suitable for administration to humans, behaves as a single unit in any of water, e.g., water at neutral pH, PBS, e.g., PBS at pH 7.4, or in a formulation in which it will be administered to patients. For purposes of calculating conjugate number, a CDP therapeutic agent conjugate molecule is a single CDP polymer with its covalently linked therapeutic agent.

Methods disclosed herein, provide for evaluating a particle, e.g., a nanoparticle, or preparation of particles, e.g., nanoparticles, wherein said particles, e.g., nanoparticles, comprise a CDP therapeutic agent (e.g., therapeutic peptide) conjugate. Generally, the method comprises providing a sample comprising a plurality of said particles, e.g., nanoparticles, determining a value for the number of CDP therapeutic agent (e.g., therapeutic peptide) conjugates in a particle, e.g., nanoparticle, in the sample, to thereby evaluate a preparation of particles, e.g., nanoparticles.

Typically the value for a particle will be a function of the values obtained for a plurality of particles, e.g., the value will be the average of values determined for a plurality of particles.

In embodiments the method further comprises comparing the determined value with a reference value. The comparison can be used in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. E.g., based on the result of the determination, or upon comparison to a reference standard, the batch from which the sample is taken can be processed, e.g., as just described.

In one embodiment, the CDP-therapeutic peptide conjugate forms or is provided as a particle (e.g., a nanoparticle) having a conjugate number described herein. By way of example, a CDP-therapeutic peptide conjugate forms, or is provided in, a nanoparticle having a conjugate number of: 1 or 2 to 25; 1 or 2 to 20; 1 or 2 to 15; 1 or 2 to 10; 1 to 3; 1 to 4:1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-therapeutic peptide conjugate is administered as a preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features, a method of evaluating a particle or a preparation of particles, wherein said particles, comprise one or a plurality of CDP therapeutic agent (e.g., therapeutic peptide) conjugate molecules, e.g., CDP-therapeutic peptide conjugates. The method comprises:

providing a sample comprising one or a plurality of said particles;

determining a value for the number of CDP conjugate molecules in a particle in said sample (the conjugate number),

thereby evaluating a preparation of particles.

In an embodiment the method comprises one or both of:

a) comparing said determined value with a reference value, e.g., a range of values, or

b) responsive to said determination, classifying said particles.

In an embodiment, the particle is a nanoparticle.

In an embodiment the method further comprises comparing said determined value with a reference standard. The reference value can be selected from a value, e.g., a range, provided herein, e.g., 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP therapeutic peptide conjugate is selected from those disclosed in herein.

In an embodiment said therapeutic peptide is selected from those disclosed herein.

In an embodiment said particle is selected from those disclosed in herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

In another aspect, the invention features, a particle, e.g., a nanoparticle, comprising one or more CDP-therapeutic peptide conjugates described herein, having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75, wherein said CDP-therapeutic agent conjugate is other than tubulysin.

As discussed above, conjugate number is defined as the number of CDP-therapeutic agent conjugate molecules that self-assemble into a particle or nanoparticle, thus

C_{J}=[CDP-therapeutic peptide conjugate]/P (or NP)

where Cj is conjugate number, [CDP-therapeutic peptide conjugate]/ is the number of CDP-therapeutic peptide conjugate molecules, and P (or NP) is a single particle (or nanoparticle).

In order to arrive and conjugate number one determines the size of a particle, e.g., by dynamic light scattering. The size should be viscosity-adjusted size. The hydrodynamic volume of a CDP-therapeutic agent conjugate, or a molecule of similar molecular weight, is determined, to provide an expected hydrodynamic volume. Comparison of the expected hydrodynamic volume for the CDP-therapeutic peptide conjugate with the volume for a particle of determined size provides conjugate number.

The determination of conjugate number is demonstrated with CRLX101, in which camptothecin is coupled to the CDP backbone. In the case of CRLX101, a number of fundamental assumptions are made in postulating nanoparticle characteristics. First, macromolecular volume estimates are based on work done with bovine serum albumin (BSA), a biological macromolecule of similar size to CRLX101 (BSA MS=67 kDa, 101 MW=66.5 kDa). It has been demonstrated that a single strand of BSA has a hydrodynamic diameter of 9.5 nm. Simple volume calculations yield a volume of 3589 nm³. Extending this to CRLX101 with an average 30 nm particle, gives a volume of 33,485 nm³. With a particle size of 5-40 nm the conjugate number is 1-30.

Polymer Polydispersity. CRLX101 molecules fall within a range of molecular weights, with molecules of varying weight providing varying contributions to the particle diameter and conjugate number. Particles could form which are made up of strands which are larger and smaller than the average. Strands may also associate to a maximum size which could be shear-limited.

Particle Shape. Particle shape is assumed to be roughly spherical, and driven by either (or both) the hydrophobic region created by the CDP-therapeutic agent conjugate, or by guest-host complexation with pendant therapeutic agent molecules making inclusion complexes with CDs from adjacent strands. One critical point of note is that as a drug product, the NPs are in a somewhat controlled environment as they are characterized. Upon administration, myriad possibilities exist for interaction with endogenous substances: inclusion complexes of circulating small molecules, metal ion complexation with the PEG subunits, etc. Any one of these are all of them in concert could dramatically alter the NP structure and function.

### Compositions of CDP-Therapeutic Peptide Conjugates

Compositions of CDP-therapeutic peptide conjugates described above may include mixtures of products. For example, the conjugation of a therapeutic peptide to a polymer may proceed in less than 100% yield, and the composition comprising the CDP-therapeutic peptide conjugate may thus also include unconjugated polymer.

Compositions of CDP-therapeutic peptide conjugates may also include CDP-therapeutic peptide conjugates that have the same polymer and the same agent, and differ in the nature of the linkage between the agent and the polymer. The CDP-therapeutic peptide conjugates may be present in the composition in varying amounts. For example, when a therapeutic peptide having a plurality of available attachment points is reacted with a polymer, the resulting composition may include more of a product conjugated via a more reactive carboxyl group, and less of a product attached via a less reactive carboxyl group.

Additionally, compositions of CDP-therapeutic peptide conjugates may include therapeutic peptides that are attached to more than one polymer chain.

### Pharmaceutical Compositions

In another aspect, the present invention provides a composition, e.g., a pharmaceutical composition, comprising a CDP-therapeutic peptide conjugate and a pharmaceutically acceptable carrier or adjuvant.

In some embodiments, a pharmaceutical composition may include a pharmaceutically acceptable salt of a compound described herein, e.g., a CDP-therapeutic peptide conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a CDP-therapeutic peptide conjugate, which may be any liquid solution compatible with the CDP-therapeutic peptide conjugate, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, and mixtures thereof.

A composition described herein may also include another component, such as an antioxidant, antibacterial, buffer, bulking agent, chelating agent, an inert gas, a tonicity agent and/or a viscosity agent.

In one embodiment, the CDP-therapeutic peptide conjugate is provided in lyophilized form and is reconstituted prior to administration to a subject. The lyophilized CDP-therapeutic peptide conjugate can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the CDP-therapeutic peptide conjugate from damage from crystal formation and the fusion process during freeze-drying. The lyoprotectant or stabilizer can be one or more of polyethylene glycol (PEG), a PEG lipid conjugate (e.g. PEG-ceramide or D-alpha-tocopheryl polyethylene glycol 1000 succinate), poly(vinyl alcohol) (PVA), poly(vinylpyrrolidone) (PVP), polyoxyethylene esters, poloxomers, Tweens, lecithins, saccharides, oligosaccharides, polysaccharides and polyols (e.g., trehalose, mannitol, sorbitol, lactose, sucrose, glucose and dextran), salts and crown ethers.

In some embodiments, the lyophilized CDP-therapeutic peptide conjugate is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution having a concentration of about 2 mg/mL to about 4 mg/mL of the CDP-therapeutic peptide conjugate. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject CDP-therapeutic peptide conjugate with a third vial containing sufficient parenteral diluent to prepare the final concentration for administration. A typical diluent is Lactated Ringer's Injection.

The final dilution of the reconstituted CDP-therapeutic peptide conjugate may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

### Routes of Administration

The pharmaceutical compositions described herein may be administered orally, parenterally (e.g., via intravenous, subcutaneous, intracutaneous, intramuscular, intraarticular, intraarterial, intrasynovial, intrastemal, intrathecal, intralesional or intracranial injection), topically, mucosally (e.g., rectally or vaginally), nasally, buccally, ophthalmically, via inhalation spray (e.g., delivered via nebulization, propellant or a dry powder device) or via an implanted reservoir.

Pharmaceutical compositions suitable for parenteral administration comprise one or more CDP-therapeutic peptide conjugate(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the CDP-therapeutic peptide conjugate then depends upon its rate of dissolution which, in turn, may depend upon crystal size and crystalline form. Alternatively, delayed absorption of a parenterally administered drug form is accomplished by dissolving or suspending the CDP-therapeutic peptide conjugate in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the CDP-therapeutic peptide conjugate, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-therapeutic peptide conjugate may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more CDP-therapeutic peptide conjugate described herein with one or more suitable non-irritating excipients which is solid at room temperature, but liquid at body temperature. The composition will therefore melt in the rectum or vaginal cavity and release the CDP-therapeutic peptide conjugate. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention.

### Dosages and Dosage Regimens

The CDP-therapeutic peptide conjugate can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the CDP-therapeutic peptide conjugate is administered to a subject at a dosage of e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m² of the therapeutic peptide. Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the CDP-therapeutic peptide conjugate is administered as a bolus infusion or intravenous push, e.g., over a period of 15 minutes, 10 minutes, 5 minutes or less. In one embodiment, the CDP-therapeutic peptide is administered in an amount such the desired dose of the agent is administered. Preferably the dose of the CDP-therapeutic peptide conjugate is a dose described herein.

In one embodiment, the subject receives 1, 2, 3, up to 10 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The CDP-therapeutic peptide conjugate can be administered as a first line therapy, e.g., alone or in combination with an additional agent or agents. In other embodiments, a CDP-therapeutic peptide is administered after a subject has developed resistance to, has filed to respond to or has relapsed after a first line therapy. The CDP-therapeutic peptide conjugate can be administered in combination with a second agent. Preferably, the CDP-therapeutic peptide is administered in combination with a second agent described herein.

### Kits

A CDP-therapeutic peptide described herein may be provided in a kit. The kit includes a CDP-therapeutic peptide conjugate described herein and, optionally, a container, a pharmaceutically acceptable carrier and/or informational material. The informational material can be descriptive, instructional, marketing or other material that relates to the methods described herein and/or the use of the CDP-therapeutic peptide conjugate for the methods described herein.

The informational material of the kits is not limited in its form. In one embodiment, the informational material can include information about production of the CDP-therapeutic peptide conjugate, physical properties of the CDP-therapeutic peptide conjugate, concentration, date of expiration, batch or production site information, and so forth. In one embodiment, the informational material relates to methods for administering the CDP-therapeutic peptide.

In one embodiment, the informational material can include instructions to administer a CDP-therapeutic peptide conjugate described herein in a suitable manner to perform the methods described herein, e.g., in a suitable dose, dosage form, or mode of administration (e.g., a dose, dosage form, or mode of administration described herein). In another embodiment, the informational material can include instructions to administer a CDP-therapeutic peptide conjugate described herein to a suitable subject, e.g., a human, e.g., a human having or at risk for a disorder described herein. In another embodiment, the informational material can include instructions to reconstitute a CDP-therapeutic peptide conjugate described herein into a pharmaceutically acceptable composition.

In one embodiment, the kit includes instructions to use the CDP-therapeutic peptide conjugate, such as for treatment of a subject. The instructions can include methods for reconstituting or diluting the CDP-therapeutic peptide conjugate for use with a particular subject or in combination with a particular chemotherapeutic agent. The instructions can also include methods for reconstituting or diluting the CDP-therapeutic peptide conjugate for use with a particular means of administration, such as by intravenous infusion.

In another embodiment, the kit includes instructions for treating a subject with a particular indication, such as a particular cancer, or a cancer at a particular stage. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a therapeutic peptide, an anthracycline, an alkylating agent, a platinum based agent, a vinca alkaloid. In another embodiment, the instructions will describe treatment of selected subjects with the CDP-therapeutic peptide conjugate. For example, the instructions can describe treatment of one or more of: a subject who has received an anticancer agent (e.g., a therapeutic peptide) and has a neutrophil count less than a standard; a subject who has moderate to severe neutropenia; a subject who has experienced one or more symptom of neuropathy from treatment with an anticancer agent, e.g., a therapeutic peptide, a vinca alkaloid, an alkylating agent, an anthracycline, a platinum-based agent or an epothilone; a subject who has experienced an infusion site reaction or has or is at risk for having hypersensitivity to treatment with an anticancer agent (e.g., a therapeutic peptide); a subject having hepatic impairment, e.g., having transaminase (ALT and/or AST levels) greater than the upper limit of normal (ULN) and/or bilirubin levels greater than ULN; a subject having hepatic impairment, e.g., ALP levels greater than the upper limit of normal (ULN), SGOT and/or SGPT levels greater the upper limit of normal (ULN) and/or bilirubin levels greater than the ULN; a subject who is currently being administered or will be administered a cytochrome P450 isoenzyme inhibitor; a subject who has experienced or is at risk for renal impairment, a subject who has or is at risk of having a gastrointestinal disorder (e.g., vomiting, nausea and/or diarrhea, e.g., associated with the administration of a chemotherapeutic agent (e.g., a therapeutic peptide)), and a subject who has or is at risk for having fluid retention and/or effusion.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a CDP-therapeutic peptide conjugate described herein and/or its use in the methods described herein. The informational material can also be provided in any combination of formats.

In addition to a CDP-therapeutic peptide conjugate described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a CDP-therapeutic peptide described herein. In such embodiments, the kit can include instructions for admixing a CDP-therapeutic peptide conjugate described herein and the other ingredients, or for using a CDP-therapeutic peptide conjugate described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-therapeutic peptide conjugate and the second therapeutic agent are in separate containers, and in another embodiment, the CDP-therapeutic peptide conjugate and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A CDP-therapeutic peptide described herein can be provided in any form, e.g., liquid, frozen, dried or lyophilized form. It is preferred that a particle described herein be substantially pure and/or sterile. When a CDP-therapeutic peptide conjugate described herein is provided in a liquid solution, the liquid solution preferably is an aqueous solution, with a sterile aqueous solution being preferred. In one embodiment, the CDP-therapeutic peptide conjugate is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a CDP-therapeutic peptide conjugate described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a CDP-therapeutic peptide conjugate described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Indications

### Cancer

The disclosed CDP-therapeutic peptide conjugates and therapeutic delivery systems comprising CDP-therapeutic peptide conjugates are useful in treating proliferative disorders, e.g., treating a tumor and metastases thereof wherein the tumor or metastases thereof is a cancer described herein. In some embodiments, wherein the agent is a diagnostic agent, the CDP-therapeutic peptide conjugates and therapeutic delivery systems comprising CDP-therapeutic peptide conjugates described herein can be used to evaluate or diagnose a cancer.

The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The CDP-therapeutic peptide conjugates, compounds or compositions described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a CDP-therapeutic peptide conjugate, compound or composition and a second therapeutic agent. Various combinations are described herein. The combination can reduce the development of tumors, reduces tumor burden, or produce tumor regression in a mammalian host.

Cancer Combination Therapy

The CDP-therapeutic peptide conjugate, compound or composition may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-therapeutic peptide conjugate, compound or composition and the at least one additional therapeutic agent can be administered simultaneously, in the same or in separate compositions, or sequentially. For sequential administration, the CDP-therapeutic peptide conjugate, compound or composition can be administered first, and the additional agent can be administered second, or the order of administration can be reversed.

In some embodiments, the CDP-therapeutic peptide conjugate, compound or composition is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the CDP-therapeutic peptide conjugate, compound or composition is administered with at least one additional therapeutic agent, such as a chemotherapeutic agent. In certain embodiments, the CDP-therapeutic peptide conjugate, compound or composition is administered in combination with one or more additional chemotherapeutic agent, e.g. with one or more chemotherapeutic agents described herein.

In some embodiments, the CDP-therapeutic peptide conjugate, compound or composition is administered in combination with a chemotherapeutic agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS),6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®) larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methyl-pentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3″-monooxime.

histone deacetylase (HDAC) inhibitors which include without limitation suberoylanilide hydroxamic acid (SAHA), [4-(2-ammo-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MN/11270B or AAJ996.

mTor inhibitors which include without limitation rapamycin (Rapamune®), and analogs and derivatives thereof. AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™)

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

In some embodiments, the CDP-therapeutic peptide conjugate, compound or composition is administered instead of another microtubule affecting agent, e.g., instead of a microtubule affecting agent as a first line therapy or a second line therapy. For example, the CDP-therapeutic peptide conjugate, compound or composition can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steroid can be administered in combination with a CDP-therapeutic peptide conjugate, compound or composition. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®. Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-therapeutic peptide conjugate, compound or composition is administered in combination with an anti-microbial (e.g., leptomycin B).

In another embodiment, the CDP-therapeutic peptide conjugate, compound or composition is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-therapeutic peptide conjugate, compound or composition is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), calcineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a CDP-therapeutic peptide conjugate, compound or composition is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary chemotherapeutic agents that may be administered in combination with a CDP-therapeutic peptide conjugate, compound or composition include, bevacizumab (Avastin®), cisplatin (Platinol®), carboplatin (Paraplat®, Paraplatin®), irinotecan (Camptosar®), floxuridine (FUDF®), 5-fluorouracil (5FU) (Adrucil®, Efudex®, Fluoroplex®), leucovorin (Wellcovorin®), capecitabine (Xeloda®), gemcitabine (Gemzar®), oxaliplatin (Eloxatin®), mitoxantrone (Novantrone®), prednisone (Delta-Dome®, Deltasone®, Liquid Pred®, Lisacort®, Meticorten®, Orasone®, Prednicen-M®, Sk-Prednisone®, Sterapred®), estramustine (Emcyt®), sunitinib (Sutent®), temsirolimus (Torisel®), sorafenib (Nexavar®), everolimus (Afinitor®), cetuximab (Erbitux®), pemetrexed (ALIMTA®), erlotinib (Tarceva®), daunorubicin (Cerubidine®, Rubidomycin®), doxorubicin (Adriamycin®), trastuzumab (Herceptin®), or tamoxifen (Nolvadex®). Exemplary combinations of agents that can be administered with a CDP-therapeutic peptide conjugate, compound or composition include, e.g., bevacizumab (Avastin®) and interferon; 5FU (Adrucil®, Efudex®, Fluoroplex®) and leucovorin (Wellcovorin®); UFT (Uftoral®) and Leucovorin (Wellcovorin®); cisplatin (Platinol®) and pemetrexed (ALIMTA®); cisplastin (Platinol®) and vinorelbine (Navelbine®); cisplastin (Platinol®) and gemcitabine (Gemzar®); cisplastin (Platinol®) and vinblastine (Velban®, Velsar®); cisplastin (Platinol®), dacarbazine (DTIC-Dome®) and vinblastine (Velban®, Velsar®); cisplastin (Platinol®), temozolomide (Methazolastone®, Temodar®) and vinblastine (Velban®, Velsar®); cisplatin (Platinol®) and 5FU (Adrucil®, Efudex®, Fluoroplex®); oxaliplatin (Eloxatin®) and irinotecan (Camptosar®); 5FU (Adrucil®, Efudex®, Fluoroplex®), irinotecan (Camptosar®), and leucovorin (Wellcovorin®); 5FU (Adrucil®, Efudex®, Fluoroplex®), irinotecan (Camptosar®), oxaliplatin (Eloxatin®), and leucovorin (Wellcovorin®); 5FU (Adrucil®, Efudex®, Fluoroplex®) and radiation; 5FU (Adrucil®, Efudex®, Fluoroplex®), radiation and cisplatin (Platinol®); oxaliplatin (Eloxatin®), 5FU (Adrucil®, Efudex®, Fluoroplex®), and leucovorin (Wellcovorin®); capecitabine (Xeloda®), oxaliplatin (Eloxatin®), and bevacizumab (Avastin®); capecitabine (Xeloda®), irinotecan (Camptosar®), and bevacizumab (Avastin®); capecitabine (Xeloda®) and bevacizumab (Avastin®); irinotecan (Camptosar®) and bevacizumab (Avastin®); cetuximab (Erbutux®) and bevacizumab (Avastin®); cetuximab (Erbutux®), irinotecan (Camptosar®) and bevacizumab (Avastin®); panitumumab (Vectibix®) and bevacizumab (Avastin®); 5FU (Adrucil®, Efudex®, Fluoroplex®), leucovorin (Wellcovorin®) and bevacizumab (Avastin®); 5FU (Adrucil®, Efudex®, Fluoroplex®), leucovorin (Wellcovorin®), oxaliplatin (Eloxatin®) and bevacizumab (Avastin®); 5FU (Adrucil®, Efudex®, Fluoroplex®), leucovorin (Wellcovorin®), irinotecan (Camptosar®) and bevacizumab (Avastin®); 5FU (Adrucil®, Efudex®, Fluoroplex®), oxaliplatin (Eloxatin®), irinotecan (Camptosar®), leucovorin (Wellcovorin®) and bevacizumab (Avastin®); and UFT (Uftoral®), irinotecan (Camptosar®) and leucovorin (Wellcovorin®).

When formulating the pharmaceutical compositions featured in the invention the clinician may utilize preferred dosages as warranted by the condition of the subject being treated. For example, in one embodiment, a CDP-therapeutic peptide conjugate, compound or composition may be administered at a dosing schedule described herein, e.g., once every one, two three four, five, or six weeks.

Also, in general, a CDP-therapeutic peptide conjugate, compound or composition, and an additional chemotherapeutic agent(s) do not have to be administered in the same pharmaceutical composition, and may, because of different physical and chemical characteristics, have to be administered by different routes. For example, the CDP-therapeutic peptide conjugate, compound or composition may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a CDP-therapeutic peptide conjugate, compound or composition is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, SN-38, camptothecin (e.g., IT-101)); and a platinum-based agent (e.g., cisplatin (Platinol®), carboplatin (Paraplat®, Paraplatin®), oxaliplatin (Eloxatin®)).

In another embodiment, the CDP-therapeutic peptide conjugate, compound or composition is administered once every two weeks in combination with one or more additional chemotherapeutic agent that is administered orally. For example, the CDP-therapeutic peptide conjugate, compound or composition can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The actual dosage of the CDP-therapeutic peptide conjugate, compound or composition and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In one embodiment, the CDP-therapeutic peptide conjugate, compound or composition can be administered at a dose that includes 0.5 to 300 mg/m² of an agent, e.g., 2.5 mg/m² to 30 mg/m², 9 to 280 mg/m², 0.5 to 100 mg/m², 0.5 to 35 mg/m², 25 to 90 mg/m². Preferably, the CDP-therapeutic peptide conjugate, compound or composition is administered at a dosage described herein.

In some embodiments, when a CDP-therapeutic peptide conjugate, compound or composition is administered in combination with one or more additional chemotherapeutic agent, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml×min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

The disclosure also encompasses a method for the synergistic treatment of cancer wherein a CDP-therapeutic peptide conjugate, compound or composition is administered in combination with an additional chemotherapeutic agent or agents.

The particular choice of polymer conjugate and anti-proliferative cytotoxic agent(s) or radiation will depend upon the diagnosis of the attending physicians and their judgment of the condition of the subject and the appropriate treatment protocol.

If the CDP-therapeutic peptide conjugate, compound or composition and the chemotherapeutic agent(s) and/or radiation are not administered simultaneously or essentially simultaneously, then the initial order of administration of the CDP-therapeutic peptide conjugate, compound or composition, and the chemotherapeutic agent(s) and/or radiation, may be varied. Thus, for example, the CDP-therapeutic peptide conjugate, compound or composition may be administered first followed by the administration of the chemotherapeutic agent(s) and/or radiation; or the chemotherapeutic agent(s) and/or radiation may be administered first followed by the administration of the CDP-therapeutic peptide conjugate, compound or composition. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-therapeutic peptide conjugate, compound or composition, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### Inflammation and Autoimmune Disease

The CDP-therapeutic peptide conjugates, particles, compositions and methods described herein may be used to treat or prevent a disease or disorder associated with inflammation. A CDP-therapeutic peptide conjugate, particle or composition described herein may be administered prior to the onset of, at, or after the initiation of inflammation. When used prophylactically, the CDP-therapeutic peptide conjugate, particle or composition is preferably provided in advance of any inflammatory response or symptom. Administration of the CDP-therapeutic peptide conjugate, particle or composition may prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components.

In another embodiment, a CDP-therapeutic peptide conjugate, particle, composition or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The CDP-therapeutic peptide conjugate, particle or composition may be used to treat chronic hepatitis infection, including hepatitis B and hepatitis C.

Additionally, a CDP-therapeutic peptide conjugate, particle, composition or method described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

Combination Therapy

In certain embodiments, a CDP-therapeutic peptide conjugate, particle or composition described herein may be administered alone or in combination with other compounds useful for treating or preventing inflammation. Exemplary anti-inflammatory agents include, for example, steroids (e.g., Cortisol, cortisone, fludrocortisone, prednisone, 6[alpha]-methylprednisone, triamcinolone, betamethasone or dexamethasone), nonsteroidal anti-inflammatory drugs (NSAIDS (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, [alpha]-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, caiprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide and cyclazocine.

Further examples of anti-inflammatory agents include deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocoitolone, fluoresone, fluorometholone, fluperolone, flupirtine, fluprednidene, fluprednisolone, fluproquazone, flurandrenolide, flurbiprofen, fluticasone, formocortal and fosfosal.

Further examples of anti-inflammatory agents include gentisic acid, glafenine, glucametacin, glycol salicylate, guaiazulene, halcinonide, halobetasol, halometasone, haloprednone, heroin, hydrocodone, hydro cortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate and myrophine.

Further examples of anti-inflammatory agents include nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papavereturn, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac. In one embodiment, a CDP-therapeutic peptide conjugate, particle or composition described herein may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, and lumiracoxib.

### Cardiovascular Disease

The disclosed methods may be useful in the prevention and treatment of cardiovascular disease. Cardiovascular diseases that can be treated or prevented using CDP-therapeutic peptide conjugates, particles, compositions and methods described herein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using CDP-therapeutic peptide conjugates, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The CDP-therapeutic peptide conjugates, particles, compositions and methods described herein may also be used for increasing HDL levels in plasma of an individual.

Yet other disorders that may be treated with CDP-therapeutic peptide conjugates, particles, compositions and methods described herein include restenosis, e.g., following coronary intervention, and disorders relating to an abnormal level of high density and low density cholesterol.

The CDP-therapeutic peptide conjugate, particle or composition can be administered to a subject undergoing or who has undergone angioplasty. In one embodiment, the CDP-therapeutic peptide conjugate, particle or composition is administered to a subject undergoing or who has undergone angioplasty with a stent placement. In some embodiments, the CDP-therapeutic peptide conjugate, particle or composition can be used as a strut of a stent or a coating for a stent.

The CDP-therapeutic peptide conjugates, particles or compositions can be used during the implantation of a stent, e.g., as a separate intravenous administration, as coating for a stent or as the strut of a stent.

Stents

The CDP-therapeutic peptide conjugates, particles or compositions described herein can be used as or be part of a stent. As used herein, the term “stent” refers to a man-made ‘tube’inserted into a natural passage or conduit in the body to prevent or counteract localized flow constriction. Types of stents include, e.g., coronary stent, urinary tract stent, urethral/prostatic stent, vascular stent (e.g., peripheral vascular stent, or stent graft), esophageal stent, duodenal stent, colonic stent, biliary stent, and pancreatic stent. Types of stents that can be used in coronary arteries include, e.g., bare-metal stent (BMS) and drug-eluting stent (DES). A coronary stent can be placed within the coronary artery during an angioplasty procedure.

Bare-Metal Stent (BMS)

In one embodiment, the CDP-therapeutic peptide conjugate, particle or composition can be used in combination with a BMS. As used herein, BMS refers to a stent without a coating that is made or a metal or combination of metals. BMS can be made from, e.g., stainless steel (e.g., BxVelocity™ stent, Express2™ stent, R Stent™, and Matrix® coronary stent), cobalt-chromium alloy (e.g., Driver® coronary stent, ML Vision® stent, and Coronnium® stent), or nickel titanium (Nitinol® stent). A CDP-therapeutic peptide conjugate, particle or composition described herein can be used as a coating of a BMS, e.g., to coat the luminal and/or abluminal surface of a BMS.

Drug-Eluting Stent (DES)

In one embodiment, the CDP-therapeutic peptide conjugate, particle or composition can be a DES or can be part of a DES. As used herein, DES refers to a stent placed into a natural passage or conduit of the body (e.g., a narrowed coronary artery) that releases (e.g., slowly releases) one or more agents to treat one or more symptoms associated with the constricted flow to the passage or conduit and/or one or more effect caused by or associated with the stent. For example, the DES can release one (or more) agent that reduces or inhibits the migration and/or proliferation of vascular smooth muscle cells (SMCs), that promotes or increases epithelialization, that reduces or inhibits a hypersensitivity reaction, that reduces or inhibits inflammation, that reduces or inhibits thrombosis, that reduces the risk of restenosis, and/or that reduces or inhibits other unwanted effects due to the stent.

One type of DES includes a stent strut and a polymer, on which an agent is loaded. Thus, in one embodiment, a CDP-therapeutic peptide conjugate, particle or composition described herein can be used in combination with other polymeric struts (e.g., other biocompatible or bioasorbable polymers). For example, a CDP-therapeutic peptide conjugate, particle or composition described herein can be coated on a polymeric strut, e.g., on the luminal and/or abluminal surface of a polymeric strut.

In another embodiment, the CDP-therapeutic peptide conjugates and therapeutic delivery systems comprising CDP-therapeutic peptide conjugates described herein can be used as a polymeric strut, with out without an additional polymer and/or agent.

In one embodiment, the rate of major adverse cardiac events (MACE) of a subject having a stent made of a CDP-therapeutic peptide conjugate, particle or composition described herein or a strut coated with a CDP-therapeutic peptide conjugate, particle or composition described herein is reduced by at least 10, 20, 30, 40, 50, 60, 70, 80, 90, 95% or more, as compared to the rate of MACE of a subject having a stent made of a different material (e.g., a metal or polymer) or a stent not coated or coated with a polymer and/or agent other than the CDP-therapeutic peptide conjugate, particle or composition. In another embodiment, the need for target vessel revascularization (TVR) of a subject having a stent made of a CDP-therapeutic peptide conjugate, particle or composition described herein or a strut coated with a CDP-therapeutic peptide conjugate, particle or composition described herein is reduced by at least 10, 20, 30, 40, 50, 60, 70, 80, 90, 95% or more, compared to the TVR of a subject having a stent made of a different material (e.g., a metal or polymer) or a stent not coated or coated with a polymer and/or agent other than the CDP-therapeutic peptide conjugate, particle or composition. In yet another embodiment, the rate for target lesion revascularization (TLR) of a subject having a stent made of a CDP-therapeutic peptide conjugate, particle or composition described herein or a strut coated with a CDP-therapeutic peptide conjugate, particle or composition described herein is reduced by at least 10, 20, 30, 40, 50, 60, 70, 80, 90, 95% or more, compared to the TLR of a subject having a stent made of a different material (e.g., a metal or polymer) or a stent not coated or coated with a polymer and/or agent other than the CDP-therapeutic peptide conjugate, particle or composition.

Polymeric Stents

Stents described herein can be made of biocompatible and/or bioabsorbable polymers. A CDP-therapeutic peptide conjugate, particle or composition described herein can be the stent, the strut of a stent or the poly-agent conjugate, particle or composition can coat a strut made of a polymeric material.

An example of a biocompatible stent is the Endeavor Rsolute® stent. This system is composed of three elements: one hydrophobic polymer (‘C10’) to retain the drug and control drug release, another polymer (‘C19’) to provide improved biocompatibility, and finally (on the outer-most side of the stent) a polyvinyl pyrrolidinone (PVP) hydrophilic polymer which increases the initial drug burst and further enhances biocompatibility. Thus, in one embodiment, the CDP-therapeutic peptide conjugate, particle or composition can be coated on an Endeavor Rsolute® stent. In other embodiments, a CDP-therapeutic peptide conjugate, particle or composition described herein can replace one or more of the elements of the Endeavor Rsolute® stent.

Bioabsorbable polymers (e.g., inert bioabsorbable polymer) can also be used in a DES, e.g., to reduce prothrombogenic potential and/or allow non-invasive imaging. In some embodiments, the bioabsorbable polymer has a degradation time of at least about 14, 21, 28, 35, 42, 49, 56, 63, 70 days.

Exemplary bioasorbable stents include, e.g., a polymeric stent (e.g., a poly-L-lactide stent, a tyrosine poly(desaminotyrosyl-tyrosine ethyl ester) carbonate stent, and a poly(anhydride ester) salicyclic acid stent). For example, Igaki-Tamai stent is constructed from a poly-L-lactic acid polymer and contains either the tyrosine kinase antagonist ST638 or paclitaxel. REVA® stent is a tyrosine poly(desaminotyrosyl-tyrosine ethyl ester) carbonate stent. It is radio-opaque and has slide and lock mechanism designed to allow for substantial reductions in stent-strut thickness. IDEAL™ stent is a poly(anhydride ester) salicyclic acid stent. Infinnium® stent is composed of two biodegradable polymers with different paclitaxel-release kinetics. Other exemplary bioasorbable stents include, e.g., BVS®, Sahajanand®, Infinnium®, BioMATRIX®, Champion®, and Infinnium®. In one embodiment, a CDP-therapeutic peptide conjugate, particle or composition described herein can be coated onto any of these bioabsorbable stents. In other embodiments, a CDP-therapeutic peptide conjugate, particle or composition described herein can replace one or more elements of one of these bioabsorbable stents.

Biosorbable Metallic Stents

The CDP-therapeutic peptide conjugates and therapeutic delivery systems comprising CDP-therapeutic peptide conjugates described herein can be used to coat a bioabsorbable metallic stent. An exemplary bioabsorbable stent is the Absorbable Metal Stent (AMS®) which is an alloy stent made of 93% magnesium and 7% rare-earth metals.

Reservoir Stents

As described herein, reservoir stents can be used, e.g., to decrease the “thickness” of the stent or reduce the unwanted effect due to microfragmentation of the polymer and/or the agent. For example, the drug can be loaded in one or more reservoirs or wells in the stent, compared to, e.g., more or less uniformly spread over the stent.

In one embodiment, a CDP-therapeutic peptide conjugate, particle or composition described herein is loaded in the reservoirs or wells located on the stent, e.g., the CDP-therapeutic peptide conjugate, particle or composition described herein is loaded in the reservoirs or wells located on the luminal side or the abluminal side of the stent. In yet another embodiment, the CDP-therapeutic peptide conjugate, particle or composition described herein is loaded in the reservoirs or wells located on both the luminal and abluminal sides of the stent.

In one embodiment, different agents (e.g., an anti-proliferation agent and a pro-endothelial agent) can be loaded into the reservoirs or wells on different sides (luminal or abluminal) of the stent, e.g., to allow for differential agent elution. In another embodiment, different agents can be loaded into adjacent reservoirs or wells of the same side (luminal or abluminal side) of the stent, e.g., to allow for dual local drug elution.

Strut

In one embodiment, the strut thickness is at least about 25, 50, 100, 150, 200, 250 μm. In another embodiment, the strut wideness is at least about 0.002, 0.004, 0.006, 0.008, or 0.01 inch. In yet another embodiment, the number of struts is at least about 4, 8, 12, 16, or 18 in its cross-section.

Various shapes of struts such as a zig zag coil, a ratchet log design, circumferential loops, etc. are known in the art and can be employed in the stents described herein.

In one embodiment, the strut can be made of a CDP-therapeutic peptide conjugate particle or composition described herein.

Combination Therapy

In one embodiment, a CDP-therapeutic peptide conjugate, particle or composition described herein may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a CDP-therapeutic peptide conjugate, particle or composition may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocainide, procainamide, encainide, flecanide, tocainide, phenyloin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltiazem, digitalis, adenosine, nickel chloride, and magnesium ions.

In another embodiment, a CDP-therapeutic peptide conjugate, particle or composition may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; antiarrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex, or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, fiunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin El and prostaglandin 12), an endothelin receptor blocking drug (such as bosentan), diltiazem, nicorandil, and nitroglycerin. Examples of cerebral protecting drugs include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of brain metabolic stimulants include amantadine, tiapride, and gamma-aminobutyric acid. Examples of anticoagulants include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of antiplatelet drugs include ticlopidine hydrochloride, dipyridamole, cilostazol, ethyl icosapentate, sarpogrelate drochloride, dilazep hydrochloride, trapidil, a nonsteroidal anti-inflammatory agent (such as aspirin), beraprostsodium, iloprost, and indobufene.

Examples of thrombolytic drugs include urokinase, tissue-type plasminogen activators (such as alteplase, tisokinase, nateplase, pamiteplase, monteplase, and rateplase), and nasaruplase. Examples of antihypertensive drugs include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of antianginal drugs include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline) trimetazidine, dipyridamole, etafenone, dilazep, trapidil, nicorandil, enoxaparin, and aspirin.

Examples of diuretics include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, andpotassiumcanrenoate), osmotic diuretics (such as isosorbide, D-mannitol, and glycerin), nonthiazide diuretics (such as meticrane, tripamide, chlorthalidone, and mefruside), and acetazolamide. Examples of cardiotonics include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate. Examples of antiarrhythmic drugs include ajmaline, pirmenol, procainamide, cibenzoline, disopyramide, quinidine, aprindine, mexiletine, lidocaine, phenyloin, pilsicainide, propafenone, flecainide, atenolol, acebutolol, sotalol, propranolol, metoprolol, pindolol, amiodarone, nifekalant, diltiazem, bepridil, and verapamil. Examples of antihyperlipidemic drugs include atorvastatin, simvastatin, pravastatin sodium, fluvastatin sodium, clinofibrate, clofibrate, simfibrate, fenofibrate, bezafibrate, colestimide, and colestyramine.

Yet other exemplary cardiovascular agents include, for example, anti-angiogenic agents and vascular disrupting agents.

### Kidney Disease

The disclosed CDP-therapeutic peptide conjugates and therapeutic delivery systems comprising CDP-therapeutic peptide conjugates are useful in treating kidney disorders, e.g., treating a kidney disorder described herein. In some embodiments, wherein the agent is a diagnostic agent, the CDP-therapeutic peptide conjugates and therapeutic delivery systems comprising CDP-therapeutic peptide conjugates described herein can be used to evaluate or diagnose a kidney disorder.

Exemplary kidney disorders include, e.g., acute kidney failure, acute nephritic syndrome, analgesic nephropathy, atheroembolic renal disease, chronic kidney failure, chronic nephritis, congenital nephrotic syndrome, end-stage renal disease, goodpasture syndrome, interstitial nephritis, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Synthesis of CDP-Histrelin Conjugate

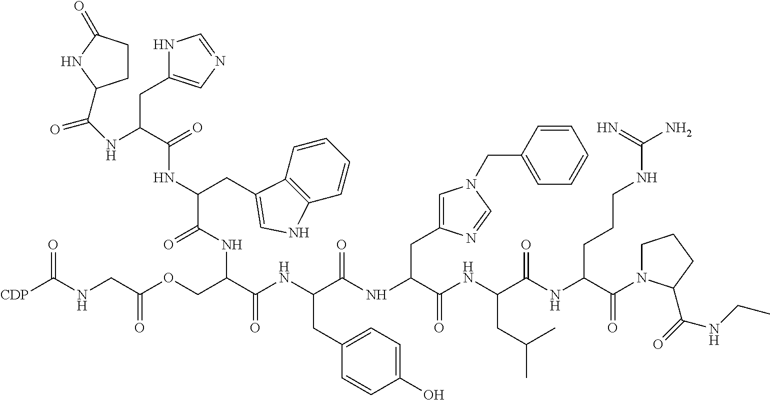

CDP (Poly-cyclodextrin-PEG) will be conjugated to histrelin by using a glycine linker that is modified on hydroxyl group on serine of histrelin. This ester linker between glycine and the therapeutic peptide can be cleaved off at high pH or by an enzyme such as estearase. ¹H NMR will be used to confirm consistency of the product. HPLC will be used to analyze the purity of the product. GPC will be used to determine the purity, molecular weight and polydispersity of the product.

In an alternative representation, the CDP-histrelin conjugate that will be formed can be represented as

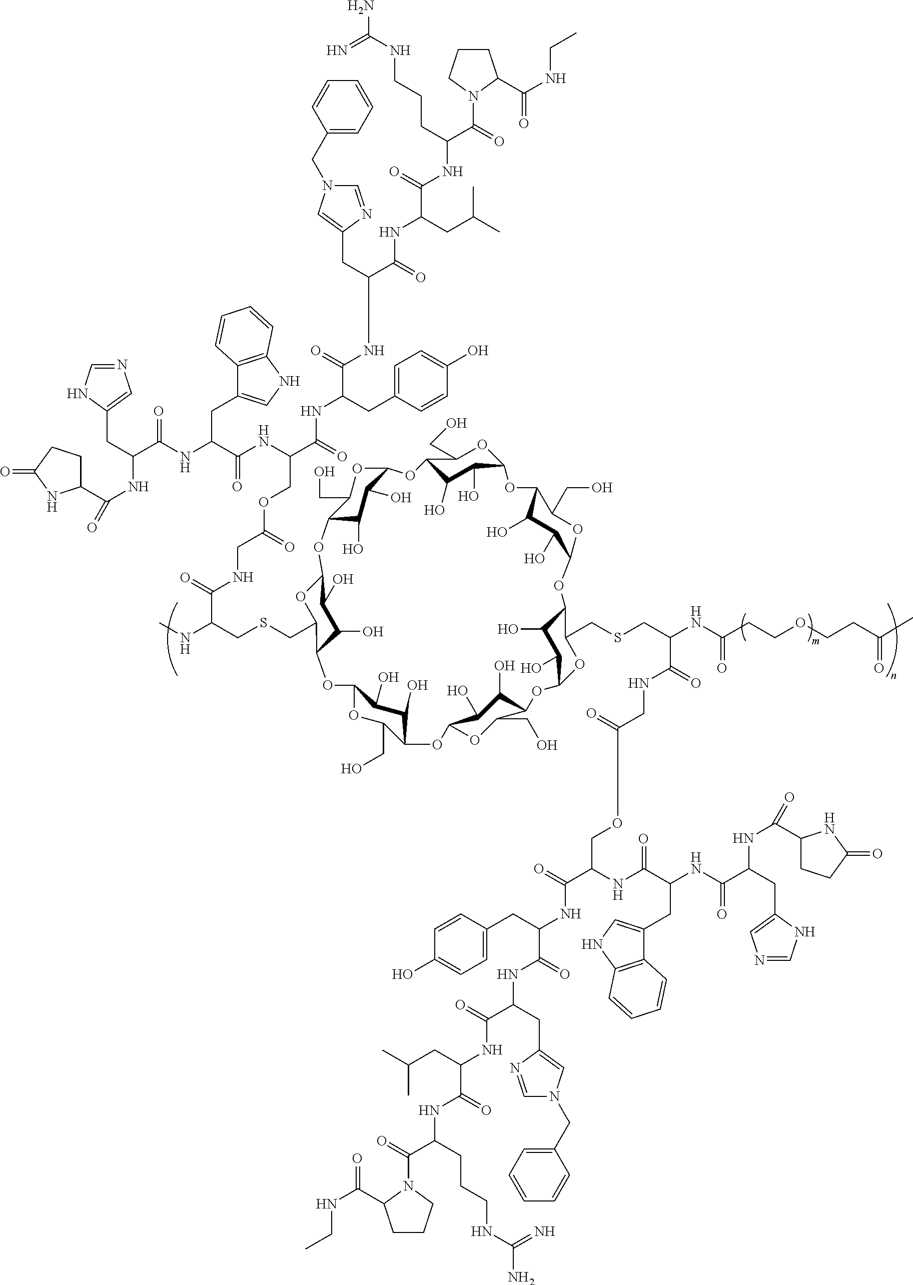

wherein the group

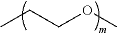

has a Mw of 5 kDa or less (e.g., 3.4 kDa) and n is at least 4, 5, 6, 7, 8, 9, 10, 11, 12, 13, 14, 15, 16, 17, 18, 19 or 20. That is, most CD molecules will be linked to more than one histrelin moiety. In some formulations the loading will be 100%. However, in some formulations the loading of the CD molecules with histrelin will not be 100%, e.g., one or more histrelins will be absent from a cysteine binding site associated with a particular CD molecule within the CDP-therapeutic peptide conjugate.

In an alternative representation, the CDP-histrelin conjugate that will be formed can be represented as

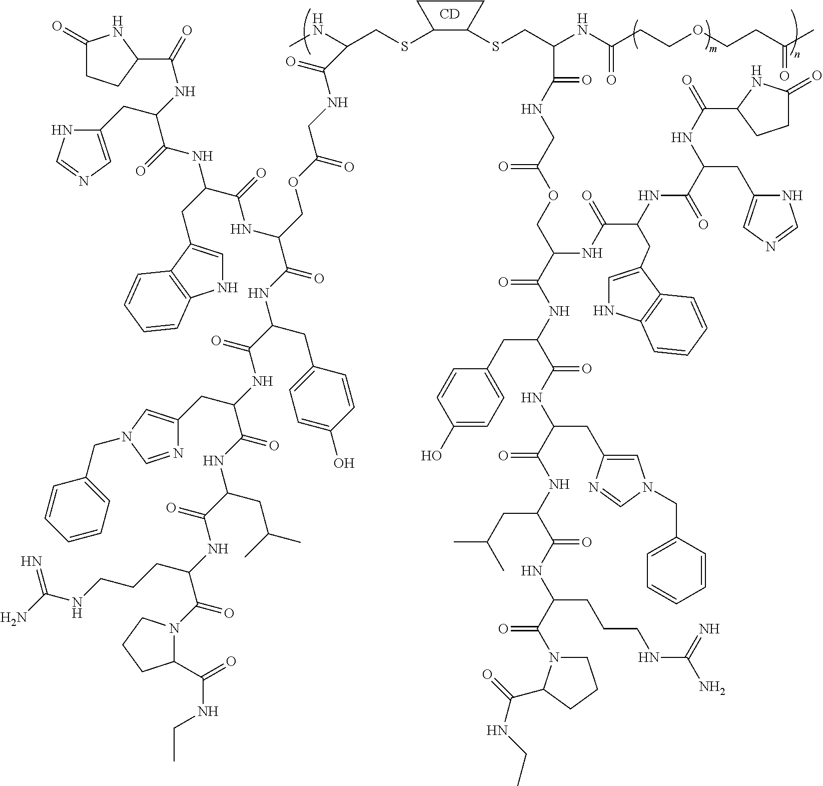

wherein the group

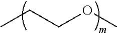

has a Mw of 5 kDa or less (e.g., 3.4 kDa) and n is at least 4, 5, 6, 7, 8, 9, 10, 11, 12, 13, 14, 15, 16, 17, 18, 19 or 20. That is, most CD molecules will be linked to more than one histrelin moiety. In some formulations the loading will be 100%. However, in some formulations the loading of the CD molecules with histrelin will not be 100%, e.g., one or more histrelins will be absent from a cysteine binding site associated with a particular CD molecule within the CDP-therapeutic peptide conjugate.

In an alternative representation, the CDP-histrelin conjugate formed will comprise the following subunit:

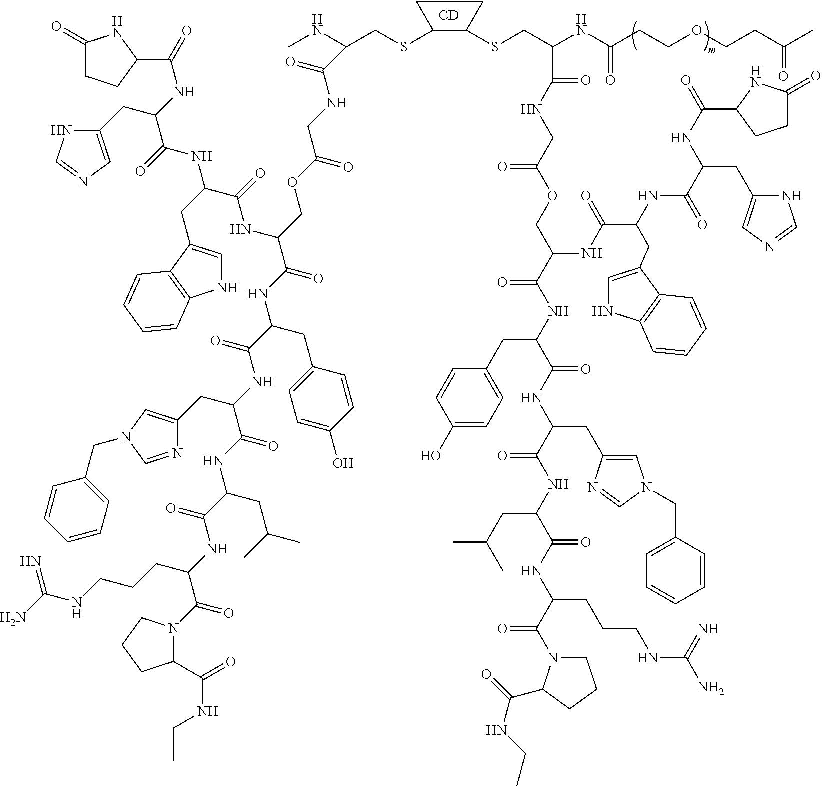

wherein the group

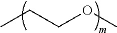

has a Mw of 5 kDa or less (e.g., 3.4 kDa).

### Example 2

### Synthesis of CDP-Nesiritide Conjugate

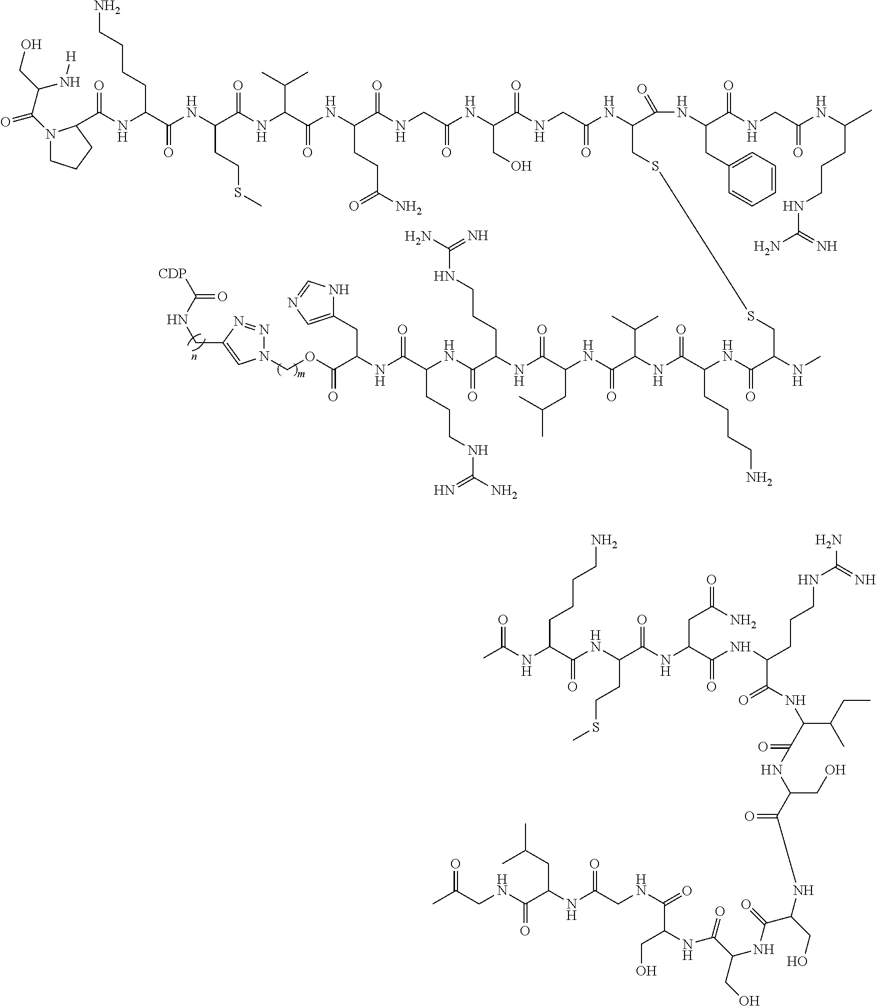

CDP will be modified at the carbonyl end group with an alkynyl functional group. Nesiritide will be functionalized with an azide group at the carbonyl end of histidine group. CDP with an alkynyl group will then be conjugated to nesiritide with an azide group to form triazole by click chemistry. This ester linker between triazole and the therapeutic peptide can be cleaved off at high pH or by an enzyme such as estearase. ¹H NMR will be used to confirm consistency of the product. HPLC will be used to analyze the purity of the product. GPC will be used to determine the purity, molecular weight and polydispersity of the product.

As in Example 1, the resultant CDP-nesiritide conjugate may be represented by any of the corresponding structures shown in Example 1, but with nesiritide moieties replacing the histrelin moieties, and with the linkage shown above. In the resulting CDP-nesiritide conjugate, most of the CD molecules will be bound to two nesiritide moieties, however, in some formulations, the loading will not be 100%.

### Example 3

### Synthesis of CDP-Thymopentin

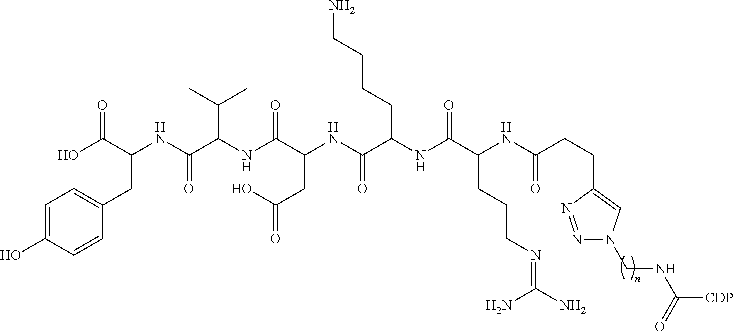

CDP will be modified at the carbonyl end group with an azide functional group. Thymopentin will be functionalized with an alkynyl group at the amino end of arginine group. CDP with an azide group will then be conjugated to thymopentin with alknyl group to form triazole by click chemistry. ¹H NMR will be used to confirm consistency of the product. HPLC will be used to analyze the purity of the product. GPC will be used to determine the purity, molecular weight and polydispersity of the product.

As in Example 1, the resultant CDP-thymopentin conjugate may be represented by any of the corresponding structures shown in Example 1, but with thymopentin moieties replacing the histrelin moieties, and with the linkage shown above. In the resulting CDP-thymopentin conjugate, most of the CD molecules will be bound to two thymopentin moieties, however, in some formulations, the loading will not be 100%.

### Example 4

### Synthesis of CDP-RWJ-800088

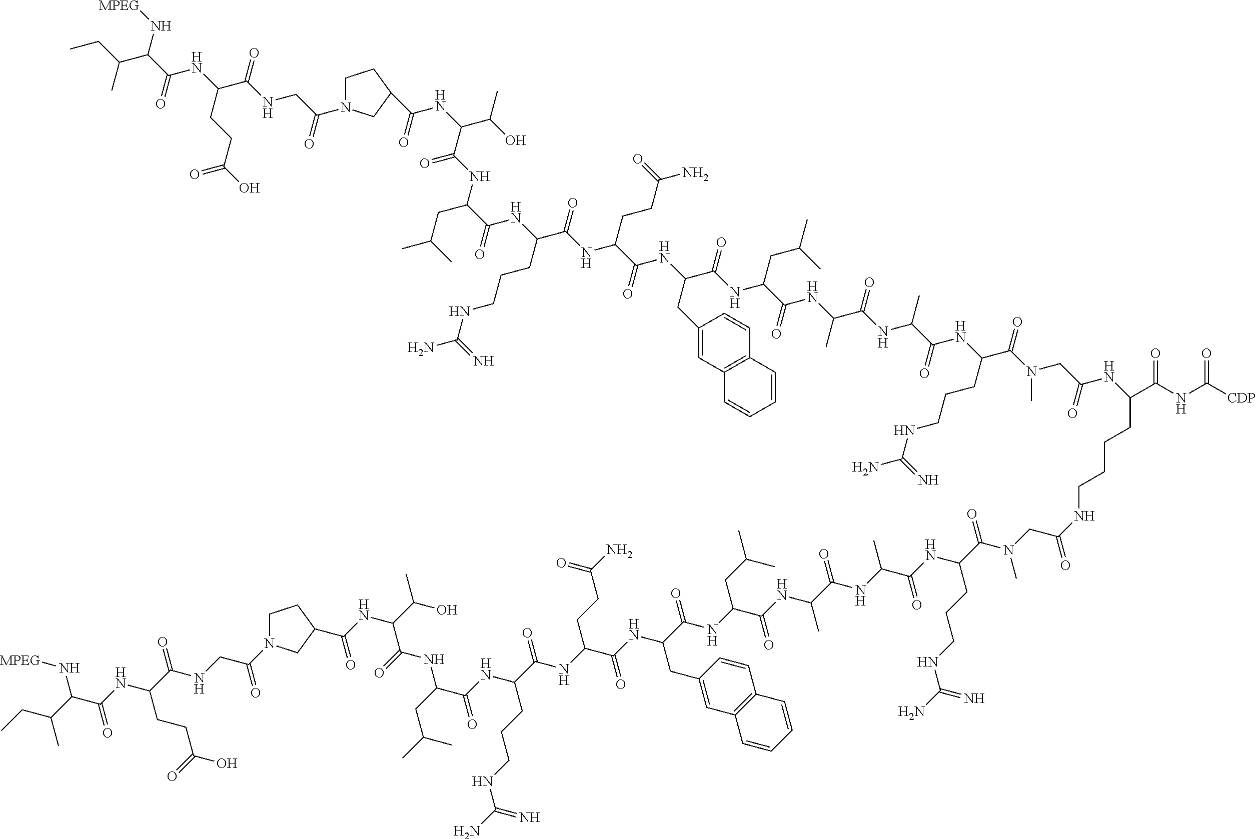

CDP will be conjugated to RWJ-800088 by formation of an amide bond between CDP and the amino end group of lysine on RWJ-800088. ¹H NMR will be used to confirm consistency of the product. HPLC will be used to analyze the purity of the product. GPC will be used to determine the purity, molecular weight and polydispersity of the product.

As in Example 1, the resultant CDP-RWJ-800088 conjugate may be represented by any of the corresponding structures shown in Example 1, but with RWJ-800088 moieties replacing the histrelin moieties, and with the linkage shown above. In the resulting CDP-RWJ-800088 conjugate, most of the CD molecules will be bound to two RWJ-800088 moieties, however, in some formulations, the loading will not be 100%.

### Example 5

### Synthesis of CDP-Alkyl-SS-Irisin Conjugate

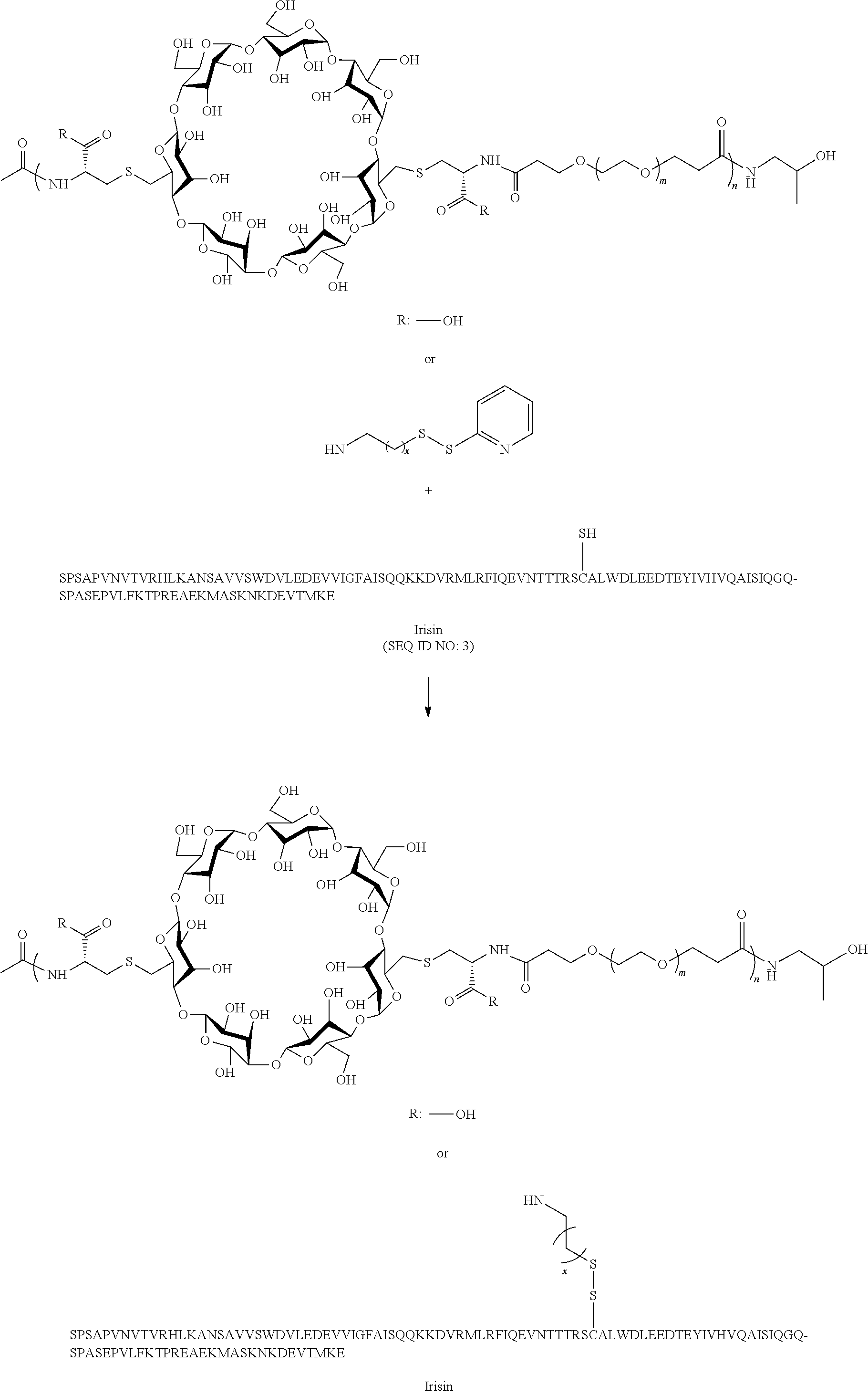

Cyclodextrin PEG polymer (CDP) will be modified with pyridine dithioAlkylamine. CDP with pyridinedithiol group will be then conjugated to Irisin by disulfide bond. This disulfide linker between CDP and Irisin can be cleaved off under reducing conditions. ¹H NMR will be used to confirm consistency of the product. HPLC shall be used to analyze the purity of the product. GPC will be used to determine the purity, molecular weight and polydispersity of the product.

### Example 6

### Synthesis of CDP-PEG-SS-Irisin Conjugate

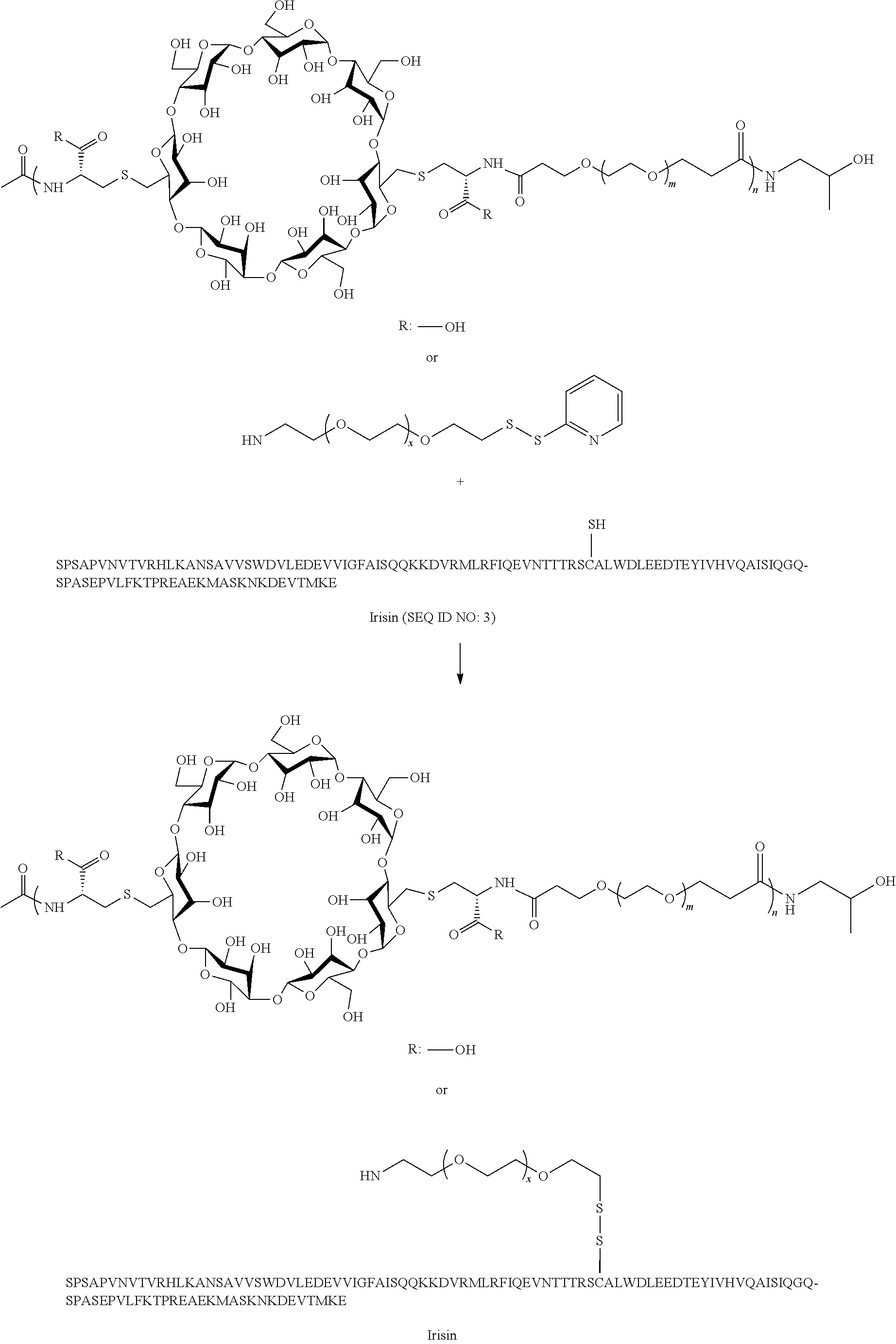

Cyclodextrin PEG polymer (CDP) will be modified with pyridine dithioPEGamine. CDP with pyridinedithiol group will be then conjugated to Irisin by disulfide bond. This disulfide linker between CDP and Irisin can be cleaved off under reducing conditions. ¹H NMR will be used to confirm consistency of the product. HPLC shall be used to analyze the purity of the product. GPC will be used to determine the purity, molecular weight and polydispersity of the product.

### Example 7

### Synthesis of CDP-Peptide-SS-Irisin Conjugate

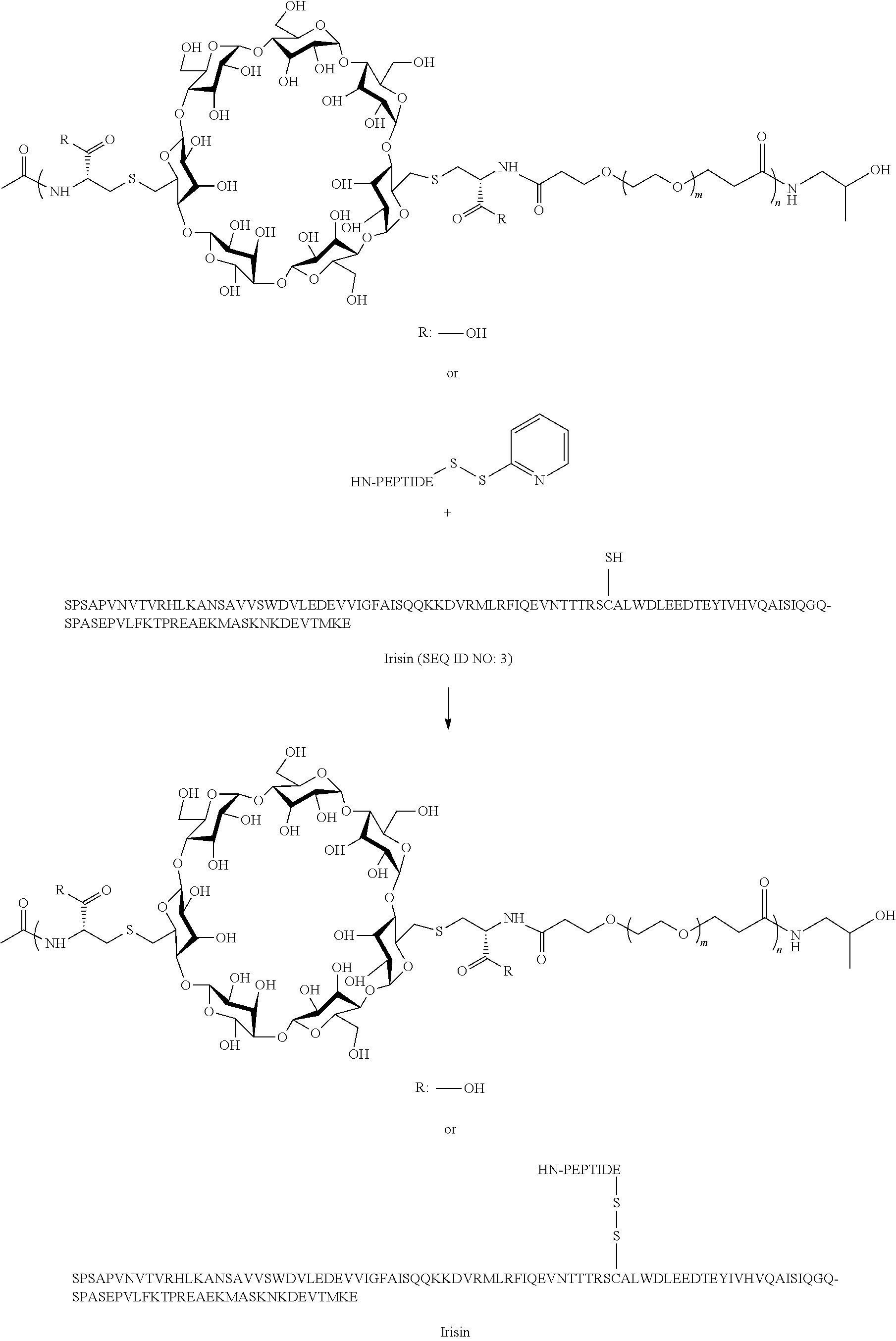

Cyclodextrin PEG polymer (CDP) will be modified with pyridine dithioPeptideamine. CDP with pyridinedithiol group will be then conjugated to Irisin by disulfide bond. This disulfide linker between CDP and Irisin can be cleaved off under reducing conditions. ¹H NMR will be used to confirm consistency of the product. HPLC shall be used to analyze the purity of the product. GPC will be used to determine the purity, molecular weight and polydispersity of the product.

### Example 8

### Synthesis of CDP-Alkyl-SS-KAI-4169 Analog Conjugate

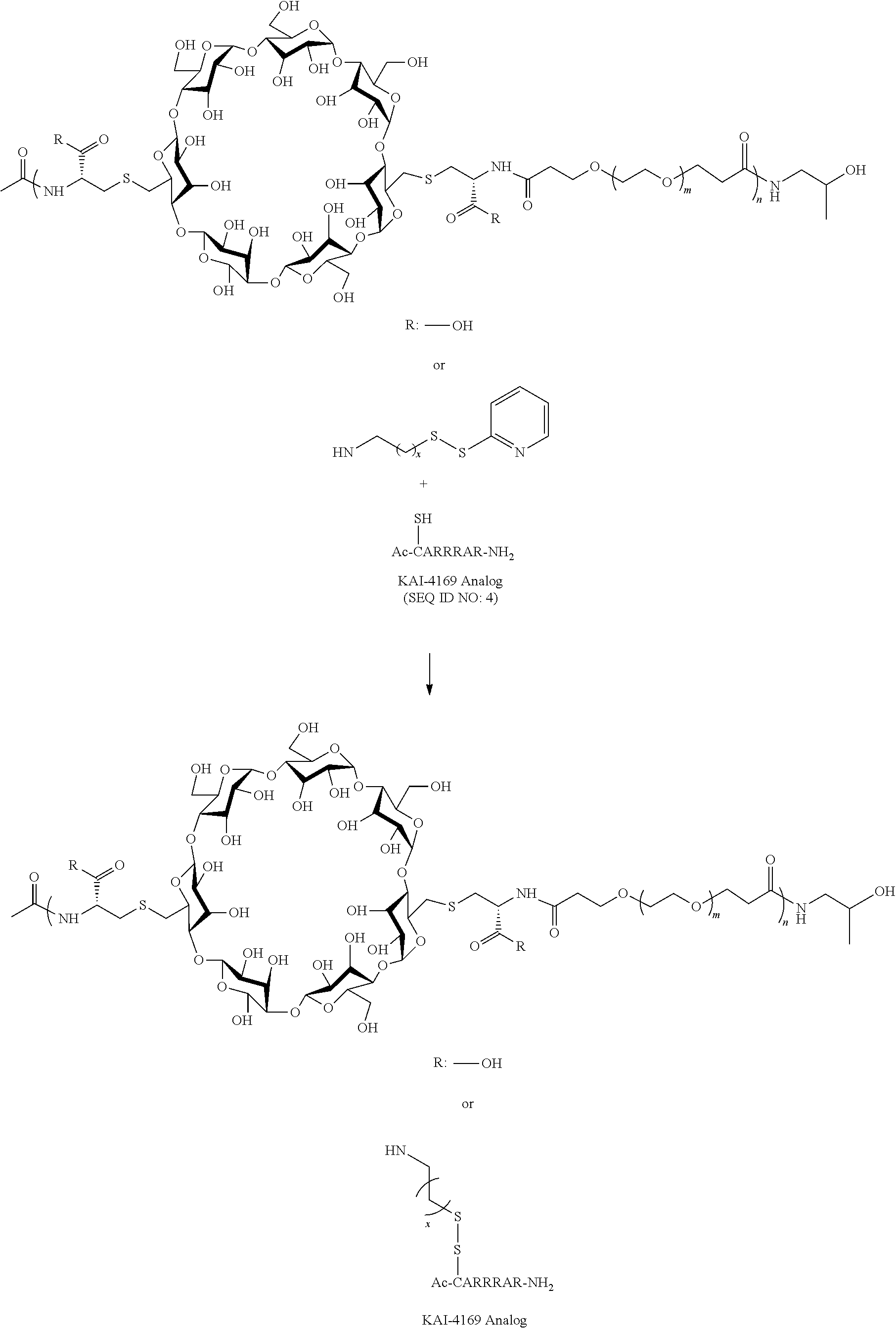

Cyclodextrin PEG polymer (CDP) will be modified with pyridine dithioAlkylamine. CDP with pyridinedithiol group will be then conjugated to KAI-4169 analog by disulfide bond. This disulfide linker between CDP and KAI-4169 analog can be cleaved off under reducing conditions. ¹H NMR will be used to confirm consistency of the product. HPLC shall be used to analyze the purity of the product. GPC will be used to determine the purity, molecular weight and polydispersity of the product.

### Example 9

### Synthesis of CDP-PEG-SS-KAI-4169 Analog Conjugate

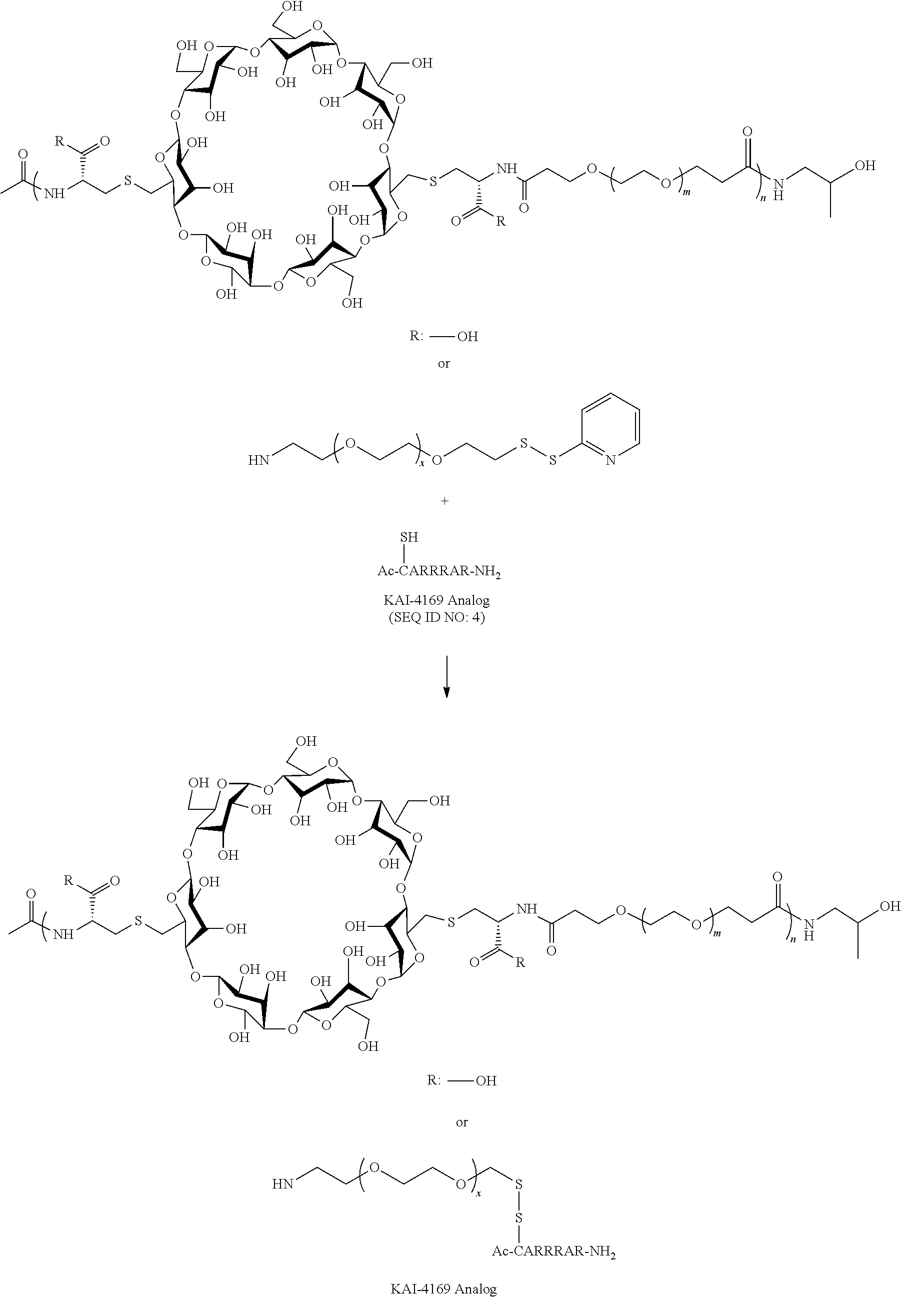

Cyclodextrin PEG polymer (CDP) will be modified with pyridine dithioPEGamine. CDP with pyridinedithiol group will be then conjugated to KAI-4169 analog by disulfide bond. This disulfide linker between CDP and KAI-4169 analog can be cleaved off under reducing conditions. ¹H NMR will be used to confirm consistency of the product. HPLC shall be used to analyze the purity of the product. GPC will be used to determine the purity, molecular weight and polydispersity of the product.

### Example 10

### Synthesis of CDP-Peptide-SS-KAI-4169 Analog Conjugate

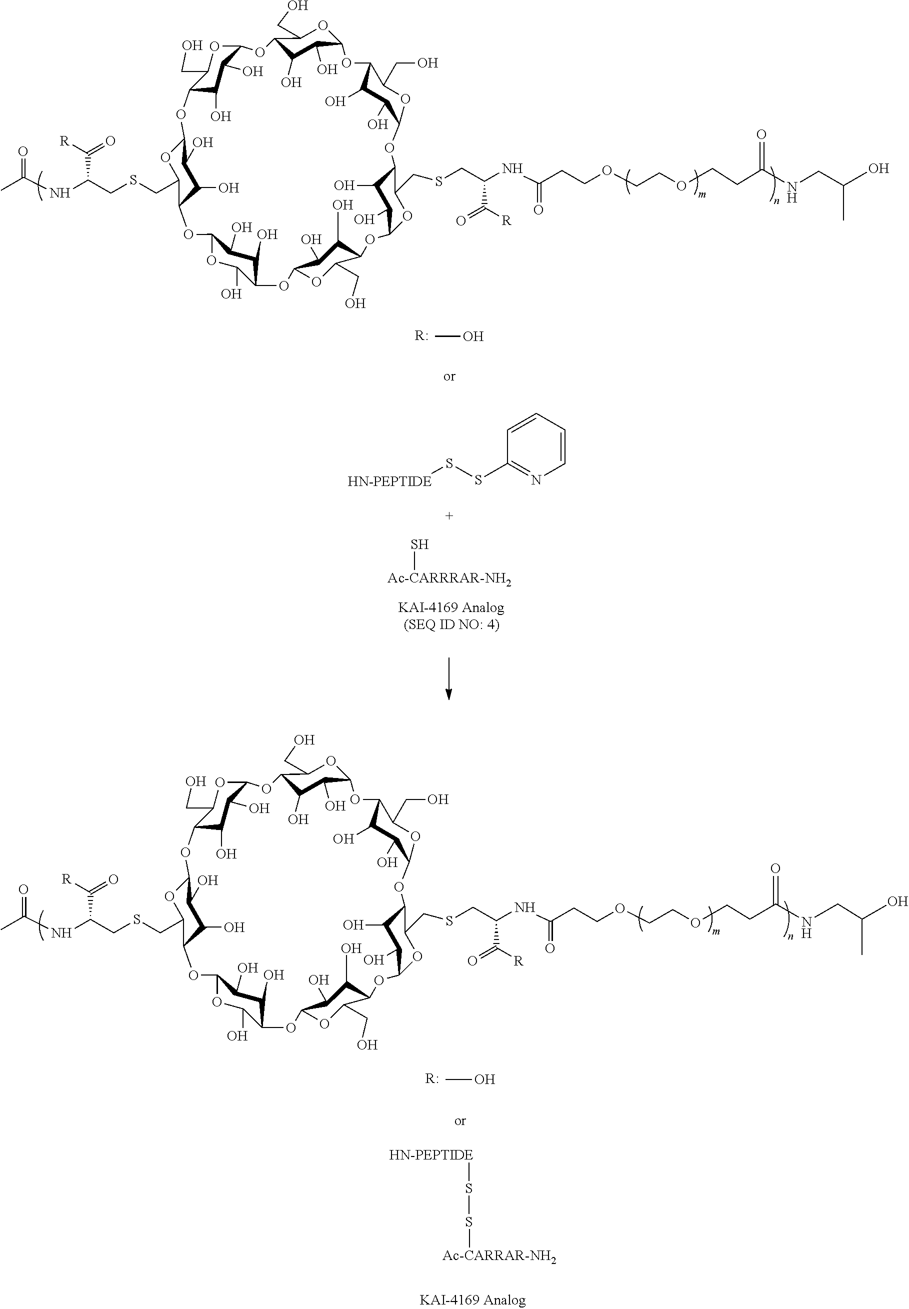

Cyclodextrin PEG polymer (CDP) will be modified with pyridine dithioPeptideamine. CDP with pyridinedithiol group will be then conjugated to KAI-4169 analog by disulfide bond. This disulfide linker between CDP and KAI-4169 analog can be cleaved off under reducing conditions. ¹H NMR will be used to confirm consistency of the product. HPLC shall be used to analyze the purity of the product. GPC will be used to determine the purity, molecular weight and polydispersity of the product.

### Example 11

### Synthesis of CDP-SS-Linacolitide (Self-Immolative Linker) Conjugate

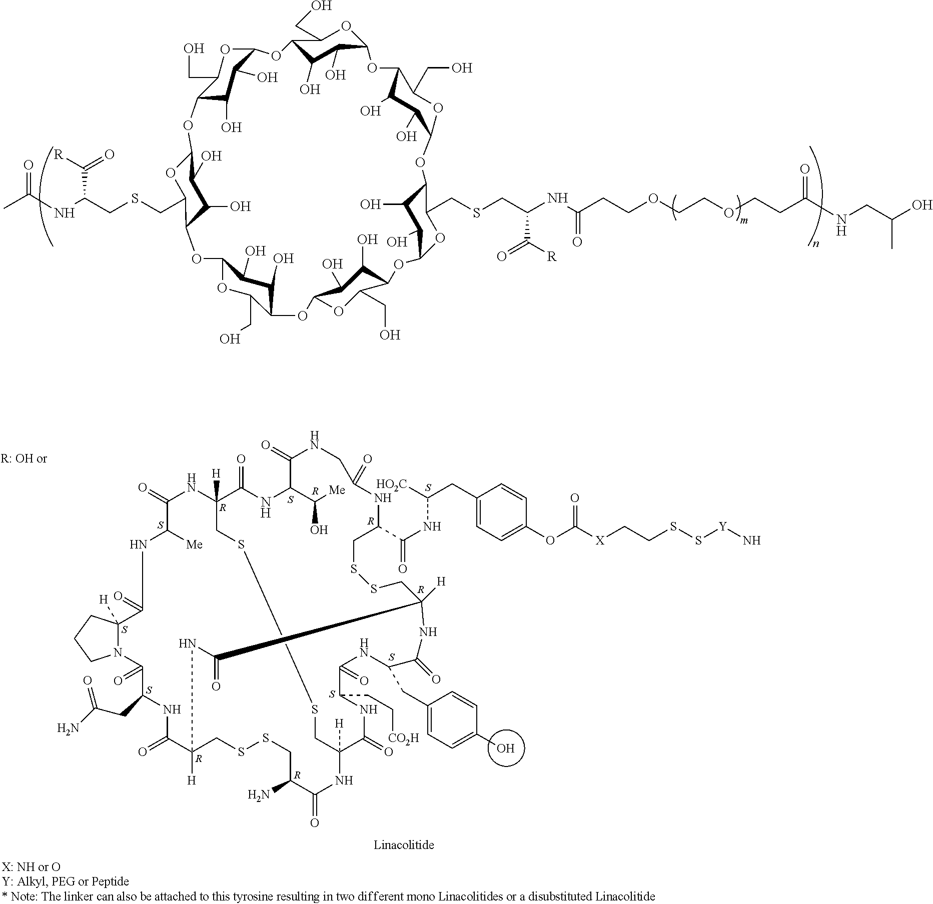

Linacolitide will be modified with disulfide linker with carbamate or carbonate bond at OH of a tyrosine. The disulfide linker can be cleaved off under reducing conditions followed by cyclization to release Linacolitide. ¹H NMR will be used to confirm consistency of the product. HPLC shall be used to analyze the purity of the product. GPC will be used to determine the purity, molecular weight and polydispersity of the product.

### Example 12

### Synthesis of CDP-PhenylSS-Linacolitide (Double Self-Immolative Linker) Conjugate

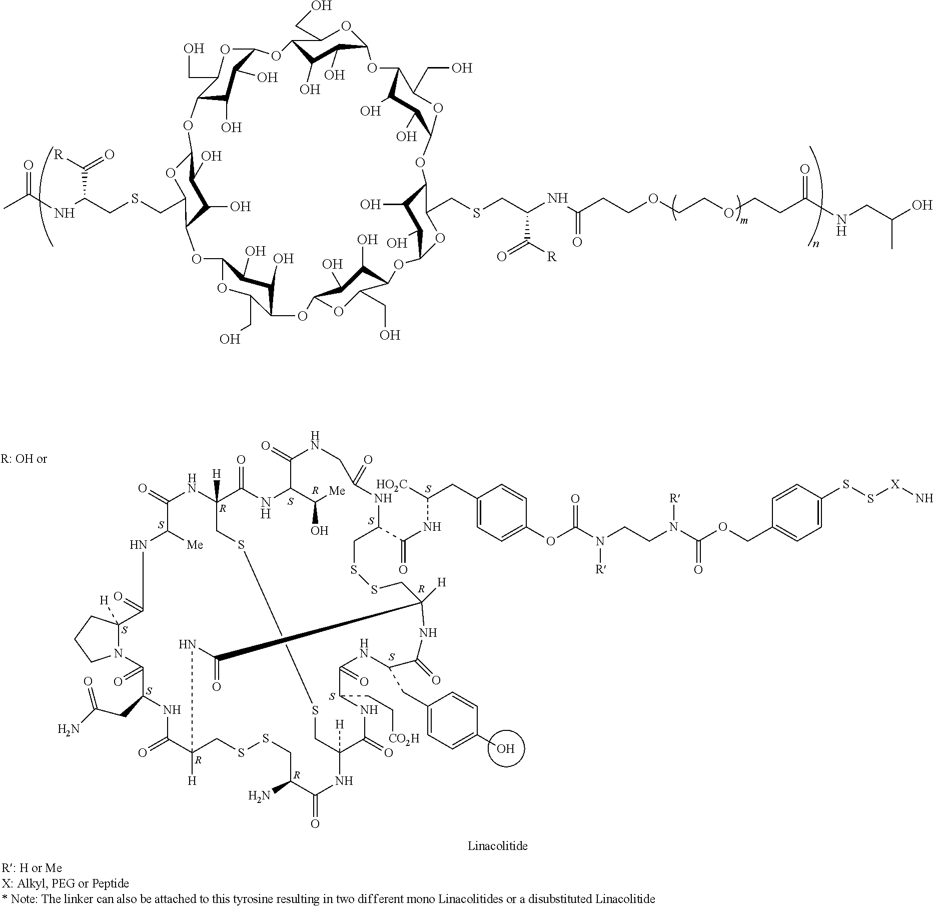

Linacolitide will be modified with disulfide linker with carbamate bond at OH of a tyrosine. The disulfide linker can be cleaved off under reducing conditions followed by 1,6 elimination of mercaptobenzyl and cyclization of diaminoethyl to release Linacolitide. ¹H NMR will be used to confirm consistency of the product. HPLC shall be used to analyze the purity of the product. GPC will be used to determine the purity, molecular weight and polydispersity of the product.

1. A method of treating a disorder in a subject in need thereof, comprising administering to the subject a CDP-therapeutic peptide conjugate in an amount effective to treat the disorder, wherein the CDP-therapeutic peptide is of the formula: 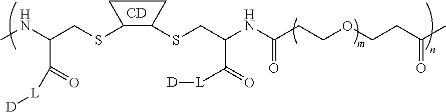
wherein each L is independently a linker or absent and each D is independently a therapeutic peptide, a prodrug thereof, or absent, and wherein the group 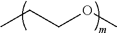
has a Mw of 5 kDa or less and n is at least 4, 5, 6, 7, 8, 9, 10, 11, 12, 13, 14, 15, 16, 17, 18, 19 or 20, provided that the polymer comprises at least one therapeutic peptide, thereby treating the subject.

2. The method of claim 1, wherein the disorder is cancer, allergies, an inflammatory disease, an auto-immune disease, a cardiovascular disease, a renal disease, or a metabolic disorder.

3. The method of claim 1, wherein the subject is a human.

4. The method of claim 1, wherein the CDP-therapeutic peptide conjugate is administered by intravenous administration.

5. (canceled)

6. (canceled)

7. (canceled)

8. A CDP-therapeutic peptide conjugate, wherein the CDP-therapeutic peptide conjugate has the following formula: 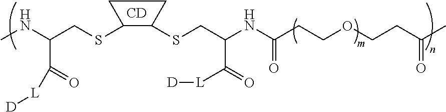
wherein each L is independently a linker or absent and each D is independently a therapeutic peptide, a prodrug thereof, or absent, and wherein the group 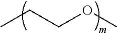
has a Mw of 5 kDa or less and n is at least 4, 5, 6, 7, 8, 9, 10, 11, 12, 13, 14, 15, 16, 17, 18, 19 or 20, provided that the polymer comprises at least one therapeutic peptide.

9. (canceled)

10. The CDP-therapeutic peptide conjugate of claim 8, wherein each L is independently an amino acid derivative.

11. (canceled)

12. The CDP-therapeutic peptide conjugate of claim 8, wherein at least a portion of the CDP is covalently attached to the therapeutic peptide through a cysteine moiety.

13. (canceled)

14. The CDP-therapeutic peptide conjugate of claim 8, wherein the linker comprises an amide bond, an ester bond, a disulfide bond, or a triazole.

15. The CDP-therapeutic peptide conjugate of claim 8, wherein the linker comprises a bond that is cleavable under physiological conditions.

16. (canceled)

17. (canceled)

18. The CDP-therapeutic peptide conjugate of claim 8, wherein at least a portion of the CDP is covalently attached to the therapeutic peptide through the carboxy terminus or an amino acid side chain of the therapeutic peptide.

19. (canceled)

20. The CDP-therapeutic peptide conjugate of claim 8, wherein the therapeutic peptides are from about 1 to about 100 weight % of the conjugate.

21. A therapeutic delivery system comprising a CDP-therapeutic peptide conjugate of claim 8, and a counter ion.

22. (canceled)

23. (canceled)

24. (canceled)

25. The therapeutic delivery system of claim 21, further comprising a surfactant.

26. (canceled)

27. (canceled)

28. (canceled)

29. An inclusion complex comprising the CDP-therapeutic peptide conjugate of claim 8.

30. An inclusion complex comprising the therapeutic delivery system of claim 21.

31. (canceled)

32. (canceled)

33. (canceled)

34. A pharmaceutical composition comprising the CDP-therapeutic peptide conjugate of claim 8.

35. A pharmaceutical composition comprising the therapeutic delivery system of claim 21.

36. (canceled)

37. A dosage form comprising the CDP-therapeutic peptide conjugate of claim 8.

38. (canceled)

39. (canceled)

40. A kit comprising the CDP-therapeutic peptide conjugate of claim 8.

41. (canceled)

42. A method of making a CDP-therapeutic peptide conjugate comprising:
providing a therapeutic peptide and a CDP; and
subjecting the therapeutic peptide and CDP to conditions that affect the covalent attachment of the therapeutic peptide to the CDP.

43. (canceled)

44. (canceled)

45. (canceled)

46. (canceled)

47. (canceled)


SEQUENCE LISTING (4 sequences)

SEQ ID NO: 1 - protein, 4 aa, Artificial Sequence
  a tetrapeptide
GFYA

SEQ ID NO: 2 - protein, 4 aa, Artificial Sequence
  a tetrapeptide
GFLG

SEQ ID NO: 3 - protein, 111 aa, homo sapiens
SPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 4 - protein, 7 aa, Artificial Sequence
  heptapeptide
CARRRAR